(19) United States
(12) Patent Application Publication
(10) Pub. No.: US 2026/0265326 A1
(43) Pub. Date: Sep. 10, 2026

(54) IL-12 FUSION PROTEIN FORMULATIONS AND METHODS OF USE THEREOF

(71) Applicant: PDS Biotechnology Corporation, Princeton, NJ (US)

(72) Inventors: Frank Bedu-Addo, Princeton, NJ (US); Gregory Conn, Princeton, NJ (US); Phonphimon Wongthida, Princeton, NJ (US); Mary Banoub, Princeton, NJ (US)

(21) Appl. No.: 19/200,532

(22) Filed: May 6, 2025

Related U.S. Application Data
(60) Provisional application No. 63/643,822, filed on May 7, 2024.

(51) Int. Cl. C07K 14/54; A61K 9/19; A61K 38/00; A61K 47/12; A61K 47/18; A61K 47/26; C07K 16/18

(52) U.S. Cl. CPC C07K 14/5434; A61K 9/19; A61K 47/12; A61K 47/183; A61K 47/26; C07K 16/18; A61K 38/00; C07K 2319/00

(57) ABSTRACT

The present invention provides stable formulations including a fusion protein including an interleukin-12 (IL-12) and an antibody. The stable formulations include a citrate buffer, an arginine stabilizer, a sugar, that is sucrose, and polysorbate 20 (PS20) as a surfactant. Also provided herein are pharmaceutical combinations including said stable formulations and one or more agents including anticancer agents, such as immune checkpoint inhibitors and/or immune modulators, such as peptides administered along with an immunogenic enhancer, such as a cationic lipid. Further, provided herein are methods of use of said formulations and combinations for treating cancer.

### CROSS REFERENCE TO RELATED APPLICATIONS

This application claims the benefit of priority under U.S.C. § 119 (e) of U.S. Provisional Patent Application Ser. No. 63/643,822 filed on May 7, 2024, the contents of which are herein incorporated by reference in its entirety.

### BACKGROUND OF THE INVENTION

### Field of the Invention

The present invention relates generally to formulations including a fusion protein including interleukin-12 (IL-12) and an antibody, and more specifically to stable formulations including said fusion protein and methods of use thereof for the treatment of cancer.

### Background Information

NHS-IL-12 fusion protein is an antibody-cytokine recombinant fusion protein (Immunocytokine) including two genetically modified human interleukin-12 (IL-12) heterodimers fused to the heavy chain (H-chain) of a phage display-derived human monoclonal IgG1 antibody (NHS76). Each IL-12 heterodimer, composed of two subunits (p35 and p40) linked through a disulfide bond, is fused to one H-chain via p35. NHS76 binds to single and double-stranded DNA such as those exposed in regions of tumor necrosis and apoptosis. The fusion protein is therefore designed to target the modified IL-12 to the tumor.

NHS-IL-12 fusion protein is highly glycosylated. The intact monomer has 26 intramolecular and 6 intermolecular disulfide bonds as well as 2 free cysteines. The molecular structure is shown in FIG. 1.

There remains an unmet need to provide formulations comprising IL-12 fusion proteins that are stable for extended periods of time.

### SUMMARY OF THE INVENTION

The present invention is based on the seminal discovery of a formulation allowing the preservation of a fusion protein including interleukin-12 (IL-12) peptide or fragment thereof and an antibody or fragment thereof for an extended period of time of at least 48 months.

In one embodiment, the present invention provides a pharmaceutical formulation including: (a) a fusion protein including an interleukin-12 (IL-12) peptide or fragment thereof and an antibody or a fragment thereof; (b) a citrate buffer; (c) a stabilizer; (d) a sugar, wherein the sugar is sucrose; and (e) a surfactant. In one aspect, the citrate buffer includes about 1 mM to 20 mM of citric acid. In some aspects, the citrate buffer includes about 10 mM citric acid. In another aspect, the stabilizer includes arginine, methionine or niacinamide. In some aspects, the stabilizer includes arginine. In one aspect, the stabilizer includes about 50 mM to 200 mM L-arginine. In one aspect, the stabilizer includes about 100 mM L-arginine. In another aspect, the sugar includes sucrose, lactose or mannitol. In some aspects, the sugar includes sucrose. In one aspect, the sugar includes about 10 mM to 200 mM sucrose. In some aspects, the sugar includes 100 mM sucrose. In another aspect, the surfactant includes polysorbate 20 (PS20), polysorbate 80 (PS80) or poloxamer 188 (P188). In some aspects, the surfactant includes PS20. In one aspect, the surfactant includes about 0.01% (w/w) to 0.5% (w/w) PS20. In some aspects, the surfactant includes about 0.05% (w/w) PS20. In one aspect, the citrate buffer includes a pH of less than about 7.0. In some aspects, the citrate buffer includes a pH from about 5.5 to 6.5. In another aspect, the citrate buffer maintains the pH and/or stability of the formulation. In one aspect, the citrate buffer maintains the integrity and/or activity of the fusion protein. In one aspect, the citrate buffer and/or the stabilizer inhibit fusion protein aggregations. In another aspect, the citrate buffer and the stabilizer improve uniformity of the formulation. In one aspect, the citrate buffer and/or the sugar maintain the isotonicity of the formulation. In another aspect, the citrate buffer and/or the PS20 surfactant improve physical and chemical stability of the formulation. In some aspects, the citrate buffer improves stability to the fusion protein as compared to formulations including a histidine buffer, a sodium phosphate buffer, a sodium acetate buffer or a succinate buffer. In other aspects, the arginine stabilizer improves stability of the fusion protein as compared to formulations including a methionine stabilizer or a niacinamide stabilizer. In another aspect, the sugar includes sucrose and improves stability of the fusion protein as compared to formulations including lactose or mannitol. In one aspect, the surfactant includes PS20 and improves stability of the fusion protein as compared to formulations including PS80 or P188. In one aspect, the pharmaceutical formulation includes (a) from about 1 mM to 20 mM citric acid; (b) from about 50 mM to 200 mM L-arginine; (c) from about 10 mM to 200 mM sucrose; and (d) from about 0.01% (w/w) to 0.5% (w/w) PS20. In some aspects, the pharmaceutical formulation includes (a) about 10 mM citric acid; (b) about 100 mM L-arginine; (c) about 100 mM sucrose; and (d) about 0.05% (w/w) PS20. In another aspect, the formulation includes a pH of about 6.0. In one aspect, the fusion protein is stable for at least 60 months at a temperature from about 2° C. to 8° C. In one aspect, the fusion protein monomer purity is about >90% for at least about 60 months at a temperature from about 2° C. to 8° C. In one aspect, fusion protein high molecular weight related substances impurity is about ≤10% for at least 60 months at a temperature ranging from about 2° C. to 8° C. In one aspect, the fusion protein is stable for at least 60 months at a temperature of about −80° C. In another aspect, the fusion protein monomer purity is about >90% for at least about 60 months at a temperature of about −80° C. In various aspects, the fusion protein high molecular weight related substances impurity is about ≤10% for at least 60 months at a temperature of about −80° C. In one aspect, the IL-12 peptide includes an IL-12 heterodimer. In another aspect, the antibody or fragment thereof includes a heavy chain of an IgG1 monoclonal antibody. In some aspects, the fusion protein includes an IL-12 heterodimer fused to a heavy chain of an IgG1 monoclonal antibody. In one aspect, the formulation is lyophilized. In some aspects, the lyophilized formulation is stable for at least 60 months. In some aspects, the lyophilized formulation is stable for about 8-15 years.

In another embodiment, the present invention provides a method of treating cancer in a subject including administering to the subject a therapeutically effective amount of the pharmaceutical formulation including a fusion protein comprising an IL-12 peptide or fragment thereof and an antibody or a fragment thereof described herein, thereby treating cancer.

In one aspect, the cancer includes an HPV-related cancer, a cancer including MUC1 expressing cancer cells, or a cancer including TARP expressing cancer cells. In some aspects, the cancer includes vulvar cancer, vaginal cancer, cervical cancer, penile cancer, anal cancer, head and neck cancer, oropharyngeal cancer, prostate cancer, breast cancer, colon cancer, gall bladder cancer, non small cell lung cancer or acute myeloid leukemia (AML).

In another aspect, the pharmaceutical formulation includes: (a) from about 1 mM to 20 mM citric acid; (b) from about 50 mM to 200 mM L-arginine; (c) from about 10 mM to 200 mM sucrose; and (d) from about 0.01% (w/w) to 0.5% (w/w) PS20. In some aspects, the pharmaceutical formulation includes: (a) about 10 mM citric acid, (b) about 100 mM L-arginine; (c) about 100 mM sucrose; and (d) about 0.05% (w/w) PS20. In one aspect, the method further includes administering an anti-cancer treatment. In one aspect, the IL-12 peptide includes an IL-12 heterodimer. In another aspect, the antibody or fragment thereof includes a heavy chain of an IgG1 monoclonal antibody. In some aspects, the fusion protein includes an IL-12 heterodimer fused to a heavy chain of an IgG1 monoclonal antibody. In some aspects, the anti-cancer treatment comprises a chemotherapeutic agent, an immunotherapeutic agent, or a targeted immunotherapeutic agent. In many aspects, the immunotherapeutic agent includes an immune checkpoint inhibitor (ICI). In one aspect, the ICI includes a PD-1 or a PD-L1 inhibitor. In another aspect, the ICI includes pembrolizumab, nivolumab, cemiplimab, dostarlimab, retifanlimab, toripalimab, atezolizumab, avelumab, durvalumab, ipilimumab, lirilumab, tremelimumab, cosibelimab or relatlimab. In many aspects, the ICI includes pembrolizumab. In another aspect, the method further includes administering an immune modulator, wherein the immune modulator includes a peptide and a cationic lipid. In some aspects, the peptide includes at least one HPV peptide, at least one mucin 1 (MUC1) peptide, or at least one T-cell receptor alternate reading frame (TARP) peptide. In one aspect, the at least one HPV peptide includes HPV16 E6 peptides, and/or HPV16 E7 peptides. In some aspects, the at least one HPV peptide includes an amino acid sequence of any of SEQ ID NOs: 5-12. In other aspects, the at least one MUC1 peptide includes an amino acid sequence of any of SEQ ID NOs: 13-37. In some aspects, the at least one TARP peptide includes an amino acid sequence of any of SEQ ID NOs: 38-49. In various aspects, the peptide comprises an animo acid sequence of any of SEQ ID NOs: 1-53. In another aspect, the cationic lipid includes 1,2-dioleoyl-3-trimethylammonium propane (DOTAP), dimethyldioctadecyl ammonium (DDA), 1,2-dioleoyl-sn-glycero-3-ethylphosphocholine (DOEPC), N-1-(2,3-dioleoyloxy) propyl-N,N,N-trimethyl ammonium chloride (DOTMA), R-DOTAP, R-DDA, R-DOEPC, R-DOTMA, S-DOTAP, S-DDA, S-DOEPC, S-DOTMA, variations or analogs thereof. In some aspects, the cationic lipid includes DOTAP. In various aspects, the cationic lipid is R-DOTAP.

In an additional embodiment, the invention provides a method of enhancing T cell infiltration and proliferation in a tumor microenvironment in a subject including administering to the subject a therapeutically effective amount of the pharmaceutical formulation including a fusion protein comprising an IL-12 and an antibody described herein, or a therapeutically effective amount of any of the pharmaceutical compositions described herein, thereby enhancing T cell infiltration and proliferation. In another aspect, the pharmaceutical formulation includes: (a) from about 1 mM to 20 mM citric acid; (b) from about 50 mM to 200 mM L-arginine; (c) from about 10 mM to 200 mM sucrose; and (d) from about 0.01% (w/w) to 0.5% (w/w) PS20. In some aspects, the pharmaceutical formulation includes: (a) about 10 mM citric acid, (b) about 100 mM L-arginine; (c) about 100 mM sucrose; and (d) about 0.05% (w/w) PS20. In one aspect, the IL-12 peptide includes an IL-12 heterodimer. In another aspect, the antibody or fragment thereof includes a heavy chain of an IgG1 monoclonal antibody. In some aspects, the fusion protein includes an IL-12 heterodimer fused to a heavy chain of an IgG1 monoclonal antibody.

In a further embodiment, the invention provides a kit including: (a) the pharmaceutical formulation including a fusion protein comprising an IL-12 and an antibody described herein, or any of the pharmaceutical compositions described herein; and (b) instructions for use.

In one aspect, the kit further includes an anti-cancer treatment and/or an immune modulator.

### BRIEF DESCRIPTION OF THE DRAWINGS

FIG. 1 is a schematic representation of the IL-12 fusion protein structure.

FIG. 2 is a graph illustrating the system suitability to measure IL-12 fusion protein formulations stability.

FIGS. 3A-3C illustrate buffers effects on IL-12 fusion protein formulations stability, the graphs compare the effects of citrate, histidine, sodium phosphate, sodium acetate, and succinate buffers. FIG. 3A is a graph illustrating IL-12 fusion protein formulations stability a TO. FIG. 3B is a graph illustrating IL-12 fusion protein formulations stability after two weeks at 5° C. FIG. 3C is a graph illustrating IL-12 fusion protein formulations stability after two weeks at 40° C. (accelerated conditions).

FIGS. 4A-4C illustrate stabilizers effects on IL-12 fusion protein formulations stability, the graphs compare the effects of arginine, methionine, and niacinamide stabilizers. FIG. 4A is a graph illustrating IL-12 fusion protein formulations stability a TO. FIG. 4B is a graph illustrating IL-12 fusion protein formulations stability after two weeks at 5° C. FIG. 4C is a graph illustrating IL-12 fusion protein formulations stability after two weeks at 40° C. (accelerated conditions).

FIGS. 5A-5C illustrate sugars effects on IL-12 fusion protein formulations stability, the graphs compare the effects of sucrose, lactose and mannitol. FIG. 5A is a graph illustrating IL-12 fusion protein formulations stability a TO. FIG. 5B is a graph illustrating IL-12 fusion protein formulations stability after two weeks at 5° C. FIG. 5C is a graph illustrating IL-12 fusion protein formulations stability after two weeks at 40° C. (accelerated conditions).

FIGS. 6A-6E illustrate stabilizers effects on IL-12 fusion protein formulations stability, the graphs compare the effects of arginine, methionine, and niacinamide stabilizers. FIG. 6A is a graph illustrating IL-12 fusion protein formulations stability a TO. FIG. 6B is a graph illustrating IL-12 fusion protein formulations stability after two weeks at 5° C. FIG. 6C is a graph illustrating IL-12 fusion protein formulations stability after two weeks at 40° C. (accelerated conditions). FIG. 6D is a graph illustrating IL-12 fusion protein formulations stability after six weeks at 5° C. FIG. 6E is a graph illustrating IL-12 fusion protein formulations stability after six weeks at 40° C. (accelerated conditions).

FIGS. 7A-7C illustrate sugars effects on IL-12 fusion protein formulations stability, the graphs compare the effects of sucrose, lactose and mannitol. FIG. 7A is a graph illustrating IL-12 fusion protein formulations stability a TO. FIG. 7B is a graph illustrating IL-12 fusion protein formulations stability after two weeks at 5° C. FIG. 7C is a graph illustrating IL-12 fusion protein formulations stability after two weeks at 40° C. (accelerated conditions).

FIGS. 8A-8E illustrate surfactant effects on IL-12 fusion protein formulations stability, the graphs compare the effects of PS20, PS80, and P188 surfactants. FIG. 8A is a graph illustrating IL-12 fusion protein formulations stability a TO. FIG. 8B is a graph illustrating IL-12 fusion protein formulations stability after one week at 5° C. FIG. 8C is a graph illustrating IL-12 fusion protein formulations stability after one week at 40° C. (accelerated conditions). FIG. 8D is a graph illustrating IL-12 fusion protein formulations stability after five weeks at 5° C. FIG. 8E is a graph illustrating IL-12 fusion protein formulations stability after five weeks at 40° C. (accelerated conditions).

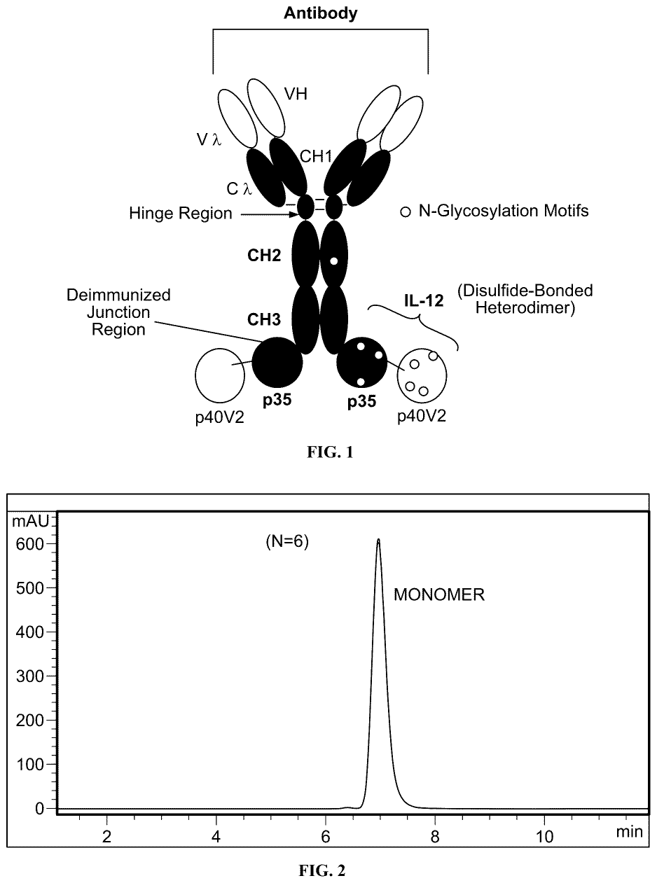

### DETAILED DESCRIPTION OF THE INVENTION

The present invention is based on the seminal discovery of a formulation allowing the preservation of a fusion protein including an interleukin-12 (IL-12) peptide or fragment thereof and an antibody or fragment thereof for an extended period of time of at least 48 months.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” in association with a numerical value is meant to include any additional numerical value reasonably close to the numerical value indicated. For example, and based on the context, the value can vary up or down by 5-10%. For example, for a value of about 100, means 90 to 110 (or any value between 90 and 110).

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

### Pharmaceutical Formulations

In one embodiment, the present invention provides a pharmaceutical formulation including: (a) a fusion protein including an interleukin-12 (IL-12) peptide or fragment thereof and an antibody or a fragment thereof; (b) a citrate buffer; (c) a stabilizer; (d) a sugar, wherein the sugar is sucrose; and (e) a surfactant.

As used herein, a pharmaceutical formulation, is meant to refer to the different chemical substances, including an active drug, that are combined to produce a final medicinal product. It may include a dosage form. A formulation includes a list of the other ingredients (excipients) are to be used in the preparation, such excipients being carefully chosen to take into account the solution including the active drug behavior under a variety of stress conditions such as freeze/thaw, temperature, shear stress among others to identify mechanisms of degradation and therefore their mitigation. Formulation considers factors such as particle size, polymorphism, pH, and solubility, as being able to influence bioavailability and hence the activity of a drug. The drug must be combined with inactive ingredients by a method that ensures that the quantity of drug present is consistent in each dosage unit. There are various formulation types, including tablets, capsules, or liquid formulations. Liquid drugs are stored in vials, IV bags, ampoules, cartridges, and prefilled syringes. As with solid formulations, liquid formulations combine the drug product with a variety of compounds to ensure stable active medication following storage. These can include solubilizers, stabilizers, buffers, tonicity modifiers, bulking agents, viscosity enhancers/reducers, surfactants, chelating agents, and/or compounds that enhance the immune response induced by the active agent in the composition (e.g., an immunogenic enhancer, immunogenic booster or immunogenic activator).

The active drug in the formulations described herein includes an IL-12 fusion protein, including an IL-12 peptide or fragment thereof and an antibody or fragment thereof. Specifically, the IL-12 peptide includes two genetically modified human interleukin-12 (IL-12) heterodimers fused to the heavy chain (H-chain) of a phage display-derived human monoclonal IgG1 antibody. The IL-12 heterodimers are disclosed in WO2007/076933, which herein is incorporated by reference in its entirety. The IgG1 antibody is disclosed in WO2000/001822, which herein is incorporated by reference in its entirety.

The IL-12 fusion protein pharmaceutical formulation described herein may be referred to as “IL-12/fusion protein pharmaceutical formulation,” “IL-12 fusion protein pharmaceutical formulation,” “interleukin-12 (IL-12)/antibody fusion protein pharmaceutical formulation,” and the like, or more generally “pharmaceutical formulation.”

Genetically modified IL-12 includes variants of human IL-12 p40 subunits (p40 variants) which have improved stability compared to wild-type IL-12 p40 proteins. In these p40 variants, the C-terminal region, which is normally sensitive to proteolytic cleavage, has been engineered to be more resistant to digestion by proteases. Specifically, p40 variants include engineered amino acid alterations in the D3 domain aimed at avoiding the creation of potential T-cell epitopes that could make the variant proteins immunogenic and trigger antibody responses in humans. As a result, p40 variants have improved properties as therapeutic agents over wild-type IL-12p40 proteins with regard to their production, formulation, and pharmacokinetics.

Accordingly, included herein is a variant of a human IL-12 p40 D3 domain (D3 variant), wherein the D3 variant has at least 85% identity with a wild-type human IL-12p40 D3 domain and includes an amino acid alteration at one or more positions corresponding to residues 258-266 of mature human IL-12 p40. Certain embodiments of the invention are based, in part, on an appreciation that an amino acid alteration or alterations of IL-12 have the particular benefit of removing the proteolytic site between Lys260 and Arg261. The amino acid alterations to one or more positions corresponding to residues 258-266 may be deletions, substitutions, or insertions. Moreover, amino acid substitutions that replace basic amino acids with non-basic amino acids can be used to create variants according to the invention.

In particular, D3 variants may include one or more amino acid substitutions at positions selected from the group consisting of Lys258, Ser259, Lys260, Arg261, Lys263, Lys264, Asp265, and Arg266. Such amino acid alterations can be used singly or in combination to induce the structural and/or functional changes described above. For example, the D3 variant can incorporate one, two, three, four or more of the following substitutions: Lys258Gln, Ser259Asp, Lys260Ala, Lys260Asn, Lys260Gln, Lys260Gly, Arg261Ala, Arg261Asp, Arg261Thr, Lys263Gly, Lys263Ser, and/or Lys264Gly.

In some aspects, the substitution is a position Lys260. The substitution may replace Lys260 with a non-basic amino acid, for example, Ala, Asn, Gln, or Gly. Further substitutions in addition to Lys260 may occur at Ser259 and Arg261. Particularly, some D3 variants incorporate substitutions Ser259Asp, Lys260Asn, and Arg261Thr. In other aspects, D3 variants incorporate substitutions Ser259Asp, Lys260Asn, Arg261Thr and Lys264Gly, while optionally deleting Lys263 and Asp265. Alternately, a D3 variant incorporates substitutions Ser259Asp, Lys260Asn, Arg261Thr, and Lys264Gly while deleting Lys263, Lys264 and Asp265.

In other aspects, a D3 variant including a substitution replacing Lys260 alternatively includes further substitutions at one or more of Lys258, Ser259, Arg261, Lys263, and Lys264. For example, a D3 variant includes the substitutions Lys258Gln, Ser259Asp, Lys260Gln, Arg261Asp, and optionally Lys263Ser and Lys264Gly. In addition to substitutions at Ser259, Lys260, and Arg261, one or more of residues corresponding to Lys263, Lys264, Asp265, and Arg266 are deleted, while in another embodiment, one or more of Lys263, Lys264, Asp265, and Arg266 are substituted with a non-basic amino acid.

In additional aspects, the substitution at Lys264 is Lys264Gly and, optionally, Lys263 and Asp265 are deleted. Other D3 variants incorporate substitutions Ser259Asp, Lys260Asn, Arg261Thr, and Lys264Gly, and optionally, deletion of residues corresponding to Lys263, Asp265 and Arg266.

It will be understood by those skilled in the art that p40 variants and active portions thereof that incorporate a D3 variant as described herein are within the scope of the disclosure. Similarly, IL-12 proteins and active portions thereof that contain a p40 variant (IL-12 variants) also are within the scope of the invention. Fusion proteins including IL-12 variants are also part of the present disclosure.

The heavy chain (H-chain) of the phage display-derived human monoclonal IgG1 antibody is meant to include the constant region and the variable region of the heavy chain. That is the variable region includes the CDRs and the framework region.

In one aspect, the IL-12 peptide includes an IL-12 heterodimer. In another aspect, the antibody or fragment thereof includes a heavy chain of an IgG1 monoclonal antibody. In some aspects, the fusion protein includes an IL-12 heterodimer fused to a heavy chain of an IgG1 monoclonal antibody.

The antibody-cytokine fusion protein (immunocytokine) comprises two disulfide-bonded IL-12 heterodimers each fused to one of the H-chains of the fully human NHS76 antibody, a human tumor necrosis monoclonal antibody derived from phage display.

The amino acid sequence of the lambda light chain of M9241 is as follows:

(SEQ ID NO: 54)
MELPVRLLVLMFWIPASLSSSELTQDPAVSVALGQTVRITCQGDS
LRSYYASWYQQKPGQAPVLVIYGKNNRPSGIPDRFSGSSSGNTAS
LTITGAQAEDEADYYCNSRDSSGNHVVFGGGTKVTVLGQPKAAPS
VTLFPPSSEELQANKATLVCLISDFYPGAVTVAWKADSSPVKAGV
ETTTPSKQSNNKYAASSYLSLTPEQWKSHKSYSCQVTHEGSTVEK
TVAPTECS,

with the V_{L} region having the amino acid sequence

(SEQ ID NO: 55)
SSSELTQDPAVSVALGQTVRITCQGDSLRSYYASWYQQKPGQAPV
LVIYGKNNRPSGIPDRFSGSSSGNTASLTITGAQAEDEADYYCNS
RDSSGNHVVFGGGTKVTVL.

The amino acid sequence of the heavy chain DI-NHSg1-M1-hu p35 is as follows:

(SEQ ID NO: 56)
MELPVRLLVLMFWIPASLSQVQLQESGPGLVKPSETLSLTCAVSG
YSISSGYYWGWIRQPPGKGLEWIGSIYHSGSTYYNPSLKSRVTIS
VDTSKNQFSLKLSSVTAADTAVYYCARGKWSKFDYWGQGTLVTVS
SASTKGPSVFPLAPSSKSTSGGTAALGCLVKDYFPEPVTVSWNSG
ALTSGVHTFPAVLQSSGLYSLSSVVTVPSSSLGTQTYICNVNHKP
SNTKVDKRVEPKSCDKTHTCPPCPAPELLGGPSVFLFPPKPKDTL
MISRTPEVTCVVVDVSHEDPEVKFNWYVDGVEVHNAKTKPREEQY
NSTYRVVSVLTVLHQDWLNGKEYKCKVSNKALPAPIEKTISKAKG
QPREPQVYTLPPSREEMTKNQVSLTCLVKGFYPSDIAVEWESNGQ
PENNYKTTPPVLDSDGSFFLYSKLTVDKSRWQQGNVFSCSVMHEA
LHNHYTQKSATATPGAANLPVATPDPGMFPCLHHSQNLLRAVSNM
LQKARQTLEFYPCTSEEIDHEDITKDKTSTVEACLPLELTKNESC
LNSRETSFITNGSCLASRKTSFMMALCLSSIYEDLKMYQVEFKTM
NAKLLMDPKRQIFLDQNMLAVIDELMQALNFNSETVPQKSSLEEP
DFYKTKIKLCILLHAFRIRAVTIDRVMSYLNAS,

with the VH region having the amino acid sequence

(SEQ ID NO: 57)
QVQLQESGPGLVKPSETLSLTCAVSGYSISSGYYWGWIRQPPGKG
LEWIGSIYHSGSTYYNPSLKSRVTISVDTSKNQFSLKLSSVTAAD
TAVYYCARGKWSKFDYWGQGTLVTVSS.

The amino acid sequence of Human P40v2 (IL-12) is as follows:

(SEQ ID NO: 59)
MCHQQLVISWFSLVFLASPLVAIWELKKDVYVVELDWYPDAPGEM
VVLTCDTPEEDGITWTLDQSSEVLGSGKTLTIQVKEFGDAGQYTC
HKGGEVLSHSLLLLHKKEDGIWSTDILKDQKEPKNKTFLRCEAKN
YSGRFTCWWLTTISTDLTFSVKSSRGSSDPQGVTCGAATLSAERV
RGDNKEYEYSVECQEDSACPAAEESLPIEVMVDAVHKLKYENYTS
SFFIRDIIKPDPPKNLQLKPLKNSRQVEVSWEYPDTWSTPHSYFS
LTFCVQVQGKDNTEGRVFTDKTSATVICRKNASISVRAQDRYYSS
SWSEWASVPCS.

The signal peptide is indicated in italic and the mutations in huP40 that removed the dibasic cleavage site KR and the potential cleavage site KK, resulting in a shortened loop, are indicated in bold (wild-type sequence KSKREKKDRV (SEQ ID NO: 60) was mutated to KDNTEGRV (SEQ ID NO:61)).

The pharmaceutical formulation includes the active drug ingredient, and a pharmaceutically acceptable carrier, diluent or excipient.

The term “active ingredient” can interchangeably refer to an “effective ingredient” and is meant to refer to any agent that is capable of inducing a sought-after effect upon administration. In one embodiment, the active ingredient includes a biologically active molecule. As used herein, the phrase “biologically active molecule” refers to a molecule that has a biological effect in a cell. In certain embodiments the active molecule may be an inorganic molecule, an organic molecule, a small organic molecule, a drug compound, a peptide, a polypeptide, such as an enzyme or transcription factor, an antibody, an antibody fragment, a peptidomimetic, a lipid, a nucleic acid such as a DNA or RNA molecule, a ribozyme, hairpin RNA, siRNA (small interfering RNAs) of varying chemistries, miRNA, siRNA-protein conjugate, an siRNA-peptide conjugate, and siRNA-antibody conjugate, an antagomir, a PNA (peptide nucleic acid), an LNA (locked nucleic acids), or a morpholino. In certain illustrative embodiments, the active agent is a polypeptide or peptide, or a fusion protein (e.g., IL-12 fusion protein).

By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carriers include, but are not limited to, liposomes, nanoparticles, ointment, micelles, microsphere, microparticle, cream, emulsion, and gel. Examples of excipients include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluents include, but are not limited to, water, alcohol, saline solution, glycol, mineral oil and dimethyl sulfoxide (DMSO).

The pharmaceutical formulation described herein includes a fusion protein including an interleukin-12 (IL-12) peptide or fragment thereof and an antibody or a fragment thereof as the active drug ingredient, and a buffer, a stabilizer, a sugar, and a surfactant as the excipients.

In one aspect, the buffer is a citrate buffer. In one aspect, the citrate buffer includes about 1 mM to 20 mM of citric acid. In some aspects, the citrate buffer includes about 10 mM citric acid. In various aspects, the buffer is not a histidine buffer, a sodium phosphate buffer, a sodium acetate buffer, nor a succinate buffer. Citric acid is required for pH control, maintenance of pH control is required for molecule stabilization. Citric acid is optimal over other buffers tested.

In one aspect, the citrate buffer pH is less than about 7.0.

Variation of pH leads to unstable formulation (aggregates). At high pH (pH 7.5) there is a tendency towards fragmentation during storage and light induced aggregation. Below pH 5.5 the acidic pH triggers protein aggregation or precipitation during stress and storage condition (except light exposure). pH can influence conformational stability by altering the ionization behavior of the charged amino acid groups on the protein surface and thus may interfere with the favorable electrostatic interaction required for maintenance of the native, folded structure.

In some aspects, the citrate buffer pH ranges from about 5.5 to 6.5. For example, the citrate buffer pH is about 5.5, 5.6, 5.7, 5.8, 5.9, 6.0, 6.1, 6.2, 6.3, 6.4, or 6.5. In one aspect, the citrate buffer has a pH of about 6.0.

In another aspect, the citrate buffer maintains the formulation pH, integrity, activity and/or stability.

In another aspect, the stabilizer includes arginine, methionine or niacinamide. In some aspects, the stabilizer includes arginine. In various aspects, the stabilizer is not a methionine stabilizer or a niacinamide stabilizer.

L-arginine affects fusion protein stability; modification of its concentration may affect stability and sensitivity to aggregation. The IL-12 fusion protein needs to be stabilized using a surfactant and stabilizer agent. L-arginine stabilizes the IL-12 fusion protein formulation preventing aggregation and fragmentation and maintaining the stability of the formulation. L-arginine is optimal over other stabilizers tested. In one aspect, the stabilizer includes about 50 mM to 200 mM L-arginine. In one aspect, the stabilizer includes about 100 mM L-arginine.

In one aspect, the citrate buffer and the arginine stabilizer inhibit fusion protein aggregations. In another aspect, the citrate buffer and the arginine stabilizer increase uniformity of the formulation. In some aspects, the citrate buffer and the arginine stabilizer provide improved stability to the fusion protein as compared to formulations using a histidine buffer, a sodium phosphate buffer, a sodium acetate buffer or a succinate buffer, or as compared to formulations using a methionine stabilizer or a niacinamide stabilizer.

In another aspect, the sugar includes sucrose, lactose or mannitol. In some aspects, the sugar includes sucrose. In various aspects, the sugar does not include lactose nor mannitol.

Sucrose is used as a tonicity agent. Additionally, sucrose acts as a cryoprotectant, antioxidant and metal chelator. The presence of cryoprotectant provides protection against freeze/thaw induced degradation of the drug substance which may undergo multiple freeze/thaw cycles during storage. In one aspect, the sugar includes about 10 mM to 200 mM sucrose. In some aspects, the sugar includes 100 mM sucrose.

In one aspect, the citrate buffer and the sucrose maintain the isotonicity of the formulation. In some aspects, the citrate buffer and sucrose provide improved stability to the fusion protein as compared to formulations using a histidine buffer, a sodium phosphate buffer, a sodium acetate buffer or a succinate buffer, or as compared to formulations using lactose or mannitol.

In another aspect, the surfactant includes polysorbate 20 (PS20), polysorbate 80 (PS80) or poloxamer 188 (P188). In various aspects, the surfactant does not include PS80 nor P188.

Variation of the concentration of polysorbate 20 may affect stability or induce, in case of slight oxidation, degradation (oxidation and aggregates), not only as content but also quality. Polysorbate 20 prevents adsorption/aggregation during freezing/thawing and allows for better stability of the formulation. IL-12 fusion protein needs to be stabilized using a surfactant and stabilizer agent. In one aspect, the surfactant includes about 0.01% (w/w) to 0.5% (w/w) PS20. In some aspects, the surfactant includes about 0.05% (w/w) PS20.

In another aspect, the citrate buffer and the PS20 surfactant increase physical and chemical stability of the formulation. In some aspects, the citrate buffer and the PS20 surfactant increase stability of the fusion protein as compared to formulations using a histidine buffer, a sodium phosphate buffer, a sodium acetate buffer or a succinate buffer, or as compared to formulations using PS80 or P188.

In another aspect, the citrate buffer maintains the pH and/or stability of the formulation. In one aspect, the citrate buffer maintains the integrity and/or activity of the fusion protein. In one aspect, the citrate buffer and/or the stabilizer inhibit fusion protein aggregations. In another aspect, the citrate buffer and the stabilizer improve uniformity of the formulation. In one aspect, the citrate buffer and/or the sugar maintain the isotonicity of the formulation. In another aspect, the citrate buffer and/or the PS20 surfactant improve physical and chemical stability of the formulation. In some aspects, the citrate buffer improves stability to the fusion protein as compared to formulations including a histidine buffer, a sodium phosphate buffer, a sodium acetate buffer or a succinate buffer. In other aspects, the arginine stabilizer improves stability of the fusion protein as compared to formulations including a methionine stabilizer or a niacinamide stabilizer. In another aspect, the sugar includes sucrose and improves stability of the fusion protein as compared to formulations including lactose or mannitol. In one aspect, the surfactant includes PS20 and improves stability of the fusion protein as compared to formulations including PS80 or P188.

In one aspect, the pharmaceutical formulation includes (a) from about 1 mM to 20 mM citric acid; (b) from about 50 mM to 200 mM L-arginine; (c) from about 10 mM to 200 mM sucrose; and (d) from about 0.01% (w/w) to 0.5% (w/w) PS20.

For example, the pharmaceutical formulation includes about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM citric acid, or more. In one aspect, the pharmaceutical formulation includes from about 1 mM to 20 mM citric acid. In various aspects, the pharmaceutical formulation includes about 10 mM citric acid.

For example, the pharmaceutical formulation includes about 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM L-arginine, or more. In one aspect, the pharmaceutical formulation includes from about 50 mM to 200 mM L-arginine. In various aspects, the pharmaceutical formulation includes about 100 mM L-arginine.

For example, the pharmaceutical formulation includes about 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM sucrose, or more. In one aspect, the pharmaceutical formulation includes from about 10 mM to 200 mM sucrose. In various aspects, the pharmaceutical formulation includes about 100 mM sucrose.

For example, the pharmaceutical formulation includes about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.11% (w/w), 0.12% (w/w), 0.13% (w/w), 0.14% (w/w), 0.15% (w/w), 0.16% (w/w), 0.17% (w/w), 0.18% (w/w), 0.19% (w/w), 0.20% (w/w), 0.21% (w/w), 0.22% (w/w), 0.23% (w/w), 0.24% (w/w), 0.25% (w/w), 0.26% (w/w), 0.27% (w/w), 0.28% (w/w), 0.29% (w/w), 0.30% (w/w), 0.31% (w/w), 0.32% (w/w), 0.33% (w/w), 0.34% (w/w), 0.35% (w/w), 0.36% (w/w), 0.37% (w/w), 0.38% (w/w), 0.39% (w/w), 0.40% (w/w), 0.41% (w/w), 0.42% (w/w), 0.43% (w/w), 0.44% (w/w), 0.45% (w/w), 0.46% (w/w), 0.47% (w/w), 0.48% (w/w), 0.49% (w/w), 0.5% (w/w) PS20, or more. In one aspect, the pharmaceutical formulation includes from about 0.01% (w/w) to 0.5% (w/w) PS20. In various aspects, the pharmaceutical formulation includes about 0.05% (w/w) PS20.

In some aspects, the pharmaceutical formulation includes (a) about 10 mM citric acid; (b) about 100 mM L-arginine; (c) about 100 mM sucrose; and (d) about 0.05% (w/w) PS20.

In another aspect, the formulation has a pH of about 6.0.

The present pharmaceutical composition is stable for extended periods of time, under various temperatures.

In various aspects, the pharmaceutical formulation has an extended shelf life, e.g., an extended stability when stored in refrigerated storage conditions. As used herein, refrigerated storage conditions generally refer to storage conditions at temperatures ranging from about 0° C. to 10° C. In various embodiments, refrigerated storage conditions include storage conditions at about 4° C.

In one aspect, the fusion protein is stable for at least 60 months at a temperature ranging from about 2° C. to 8° C. In another aspect, the fusion protein is stable for about 8-15 years at a temperature ranging from about 2° C. to 8° C.

For example, the pharmaceutical formulation is stable for about 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 61 months, 62 months, 63 months, 64 months, 65 months, 66 months, 67 months, 68 months, 69 months, 70 months, 71 months, 72 months, or more.

For example, the pharmaceutical formulation is stable for about 4 years, 5 years, 6 years, 7 years, 8 years, 9 years, 10 years, 11 years, 12 years, 13 years, 14 years, 15 years, 16 years or more. For example, the pharmaceutical formulation is stable for about 4-16 years, 5-16 years, 6-16 years, 7-16 years, 8-16 years, 9-16 years, 10-16 years, 11-16 years, 12-16 years, 13-16 years, 14-16 years, 15-16 years or more, or any ranges in between (e.g., 4-5 years, 5-10 years, 8-10 years, and the like).

For example, the pharmaceutical composition is stable at a temperature of about 0° C., 1° C., 2° C., 3° C., 4° C., 5° C., 6° C., 7° C., 8° C., 9° C., 10° C., or more.

For example, the pharmaceutical composition is stable for about 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 61 months, 62 months, 63 months, 64 months, 65 months, 66 months, 67 months, 68 months, 69 months, 70 months, 71 months, 72 months, or more, at a temperature of about 0° C., 1° C., 2° C., 3° C., 4° C., 5° C., 6° C., 7° C., 8° C., 9° C., 10° C., or more.

For example, the pharmaceutical formulation is stable for about 4 years, 5 years, 6 years, 7 years, 8 years, 9 years, 10 years, 11 years, 12 years, 13 years, 14 years, 15 years, 16 years or more, at a temperature of about 0° C., 1° C., 2° C., 3° C., 4° C., 5° C., 6° C., 7° C., 8° C., 9° C., 10° C., or more.

For example, the pharmaceutical formulation is stable for about 4-16 years, 5-16 years, 6-16 years, 7-16 years, 8-16 years, 9-16 years, 10-16 years, 11-16 years, 12-16 years, 13-16 years, 14-16 years, 15-16 years or more, or any ranges in between, at a temperature of about 0° C., 1° C., 2° C., 3° C., 4° C., 5° C., 6° C., 7° C., 8° C., 9° C., 10° C., or more

In another aspect, the fusion protein is stable for at least 60 months at a temperature of about −80° C.

For example, the pharmaceutical composition is stable for about 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 61 months, 62 months, 63 months, 64 months, 65 months, 66 months, 67 months, 68 months, 69 months, 70 months, 71 months, 72 months, or more.

For example, the pharmaceutical composition is stable at a temperature of about −70° C., −71° C., −72° C., −73° C., −74° C., −75° C., −76° C., −77° C., −78° C., −79° C., −80° C., or less.

For example, the pharmaceutical composition is stable for about 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 61 months, 62 months, 63 months, 64 months, 65 months, 66 months, 67 months, 68 months, 69 months, 70 months, 71 months, 72 months, or more, at a temperature of about −70° C., −71° C., −72° C., −73° C., −74° C., −75° C., −76° C., −77° C., −78° C., −79° C., −80° C., or less.

For example, the pharmaceutical formulation is stable for about 4 years, 5 years, 6 years, 7 years, 8 years, 9 years, 10 years, 11 years, 12 years, 13 years, 14 years, 15 years, 16 years or more, at a temperature of about −70° C., −71° C., −72° C., −73° C., −74° C., −75° C., −76° C., −77° C., −78° C., −79° C., −80° C., or less.

For example, the pharmaceutical formulation is stable for about 4-16 years, 5-16 years, 6-16 years, 7-16 years, 8-16 years, 9-16 years, 10-16 years, 11-16 years, 12-16 years, 13-16 years, 14-16 years, 15-16 years or more, or any ranges in between, at a temperature of about −70° C., −71° C., −72° C., −73° C., −74° C., −75° C., −76° C., −77° C., −78° C., −79° C., −80° C., or less.

In one aspect, the fusion protein monomer purity is about >90% for at least about 60 months at a temperature ranging from about 2° C. to 8° C.

For example, the fusion protein monomer purity is about 90%, 91%, 92%, 93%, 94%, 95%, 96%, 97%, 98%, 99%, 99.5%, or more.

For example, the fusion protein monomer purity is about 90%, 91%, 92%, 93%, 94%, 95%, 96%, 97%, 98%, 99%, 99.5%, or more, for about 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 61 months, 62 months, 63 months, 64 months, 65 months, 66 months, 67 months, 68 months, 69 months, 70 months, 71 months, 72 months, or more, at a temperature of about 0° C., 1° C., 2° C., 3° C., 4° C., 5° C., 6° C., 7° C., 8° C., 9° C., 10° C., or more.

In another aspect, the fusion protein monomer purity is about >90% for at least about 60 months at a temperature of about −80° C.

For example, the fusion protein monomer purity is about 90%, 91%, 92%, 93%, 94%, 95%, 96%, 97%, 98%, 99%, 99.5%, or more, for about 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 61 months, 62 months, 63 months, 64 months, 65 months, 66 months, 67 months, 68 months, 69 months, 70 months, 71 months, 72 months, or more, at a temperature of about −70° C., −71° C., −72° C., −73° C., −74° C., −75° C., −76° C., −77° C., −78° C., −79° C., −80° C., or less.

In one aspect, fusion protein high molecular weight related substances impurity is about <10% for at least 60 months at a temperature ranging from about 2° C. to 8° C.

For example, high molecular weight related substances impurity is about 10%, 9%, 8%, 7%, 6%, 5%, 4%, 3%, 2%, 1%, 0.5%, or less.

For example, high molecular weight related substances impurity is about 10%, 9%, 8%, 7%, 6%, 5%, 4%, 3%, 2%, 1%, 0.5%, or less, for about 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 61 months, 62 months, 63 months, 64 months, 65 months, 66 months, 67 months, 68 months, 69 months, 70 months, 71 months, 72 months, or more, at a temperature of about 0° C., 1° C., 2° C., 3° C., 4° C., 5° C., 6° C., 7° C., 8° C., 9° C., 10° C., or more.

In another aspect, fusion protein high molecular weight related substances impurity is about ≤10% for at least 60 months at a temperature of about −80° C.

For example, high molecular weight related substances impurity about 10%, 9%, 8%, 7%, 6%, 5%, 4%, 3%, 2%, 1%, 0.5%, or less, for about 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 61 months, 62 months, 63 months, 64 months, 65 months, 66 months, 67 months, 68 months, 69 months, 70 months, 71 months, 72 months, or more, at a temperature of about −70° C., −71° C., −72° C., −73° C., −74° C., −75° C., −76° C., −77° C., −78° C., −79° C., −80° C., or less.

In one aspect, the formulation is lyophilized.

As used herein, the term “lyophilization” is meant to refer to “freeze-drying,” or “cryodesiccation,” a low temperature dehydration process that involves freezing the pharmaceutical formulation and lowering pressure, thereby removing the ice by sublimation. This is in contrast to dehydration by most conventional methods that evaporate water using heat. Because of the low temperature used in processing, the rehydrated product retains many of its original qualities. When the product to be dried is a liquid, as often seen in pharmaceutical applications, the properties of the final product are optimized by the combination of excipients (i.e., inactive ingredients). Primary applications of freeze-drying preservation.

Freeze-drying is generally used for pharmaceutical formulation to increase the shelf life of the products, such as biologics, and other injectables. By removing the water from the material and sealing the material in a glass vial, the material can be easily stored, shipped, and later reconstituted to its original form for injection. Another example from the pharmaceutical industry is the use of freeze-drying to produce tablets or wafers, the advantage of which is less excipient as well as a rapidly absorbed and easily administered dosage form. Freeze-dried pharmaceutical products are produced as lyophilized powders for reconstitution in vials and more recently in prefilled syringes for self-administration by a patient.

In some aspects, the lyophilized formulation is stable for at least 60 months.

For example, the lyophilized formulation is stable for about 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, 60 months, 61 months, 62 months, 63 months, 64 months, 65 months, 66 months, 67 months, 68 months, 69 months, 70 months, 71 months, 72 months, or more.

In some aspects, the lyophilized formulation is stable for about 60 months.

In some aspects, the lyophilized formulation is stable for about 8-15 years.

For example, the lyophilized pharmaceutical formulation is stable for about 4 years, 5 years, 6 years, 7 years, 8 years, 9 years, 10 years, 11 years, 12 years, 13 years, 14 years, 15 years, 16 years or more.

For example, the lyophilized pharmaceutical formulation is stable for about 4-16 years, 5-16 years, 6-16 years, 7-16 years, 8-16 years, 9-16 years, 10-16 years, 11-16 years, 12-16 years, 13-16 years, 14-16 years, 15-16 years or more, or any ranges in between.

The pharmaceutical formulation may be preserved as a lyophilized powder or as a liquid formulation. The pharmaceutical formulation (lyophilized and liquid versions) may be preserved, at a temperature of about −70° C., −71° C., −72° C., −73° C., −74° C., −75° C., −76° C., −77° C., −78° C., −79° C., −80° C., or less; or at a temperature of about 0° C., 1° C., 2° C., 3° C., 4° C., 5° C., 6° C., 7° C., 8° C., 9° C., 10° C., or more.

### Methods of Use

In one embodiment, the present invention provides a method of treating cancer including administering to a subject a therapeutically effective amount of the pharmaceutical formulation including a fusion protein comprising an IL-12 peptide or a fragment thereof and an antibody or a fragment thereof described herein, thereby treating cancer.

The term “subject” as used herein refers to any individual or patient to which the subject methods are performed. Generally, the subject is human, although as will be appreciated by those in the art, the subject may be a non-human animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” or “therapy” and refers to 1) therapeutic treatments or measures that cure, slow down, lessen symptoms of, and/or halt progression of a diagnosed pathologic conditions or disorder, and/or 2) prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of cancer). Such amount should be sufficient to treat cancer in the subject. The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intramuscular, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

In some aspects administration can be in combination with one or more additional therapeutic agents. The phrases “combination therapy”, “combined with” and the like refer to the use of more than one medication or treatment simultaneously to increase the response. The formulations including a fusion protein comprising an IL-12 peptide or a fragment thereof and an antibody or a fragment thereof described herein might for example be used in combination with other drugs or treatment in use to treat cancer. Specifically, the administration of the formulations including a fusion protein comprising an IL-12 peptide or a fragment thereof and an antibody or a fragment thereof described herein to a subject can be in combination with an anti-cancer treatment and/or an immune modulator. Such therapies can be administered prior to, simultaneously with, or following administration of the formulations of the present invention.

The methods described herein include methods of treating cancer in a subject.

Cancer is a group of diseases involving abnormal cell growth with the potential to invade or spread to other parts of the body. In 2015, about 90.5 million people had cancer, about 14.1 million new cases occur a year and it caused about 8.8 million deaths (15.7% of deaths). The most common types of cancer in males are lung cancer, prostate cancer, colorectal cancer and stomach cancer. In females, the most common types are breast cancer, colorectal cancer, lung cancer and cervical cancer. The term “cancer” refers to a group of diseases characterized by abnormal and uncontrolled cell proliferation starting at one site (primary site) with the potential to invade and to spread to other sites (secondary sites, metastases) which differentiate cancer (malignant tumor) from benign tumor. Virtually all organs can be affected, leading to more than 100 types of cancer that can affect humans. Cancers can result from many causes including genetic predisposition, viral infection, exposure to ionizing radiation, exposure to environmental pollutant, tobacco and or alcohol use, obesity, poor diet, lack of physical activity or any combination thereof.

As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, pancreatic, brain, and head and neck cancers, melanoma, sarcoma, multiple myeloma, leukemia, lymphoma, and the like.

Exemplary cancers described by the national cancer institute include: Acute Lymphoblastic Leukemia, Adult; Acute Lymphoblastic Leukemia, Childhood; Acute Myeloid Leukemia, Adult; Acute Myeloid Leukemia, Childhood; Adrenocortical Carcinoma, Adult; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer, Adult; Bladder Cancer, Childhood; Bone Cancer, Osteosarcoma/Malignant Fibrous Histiocytoma; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

In one aspect, the cancer includes an HPV-related cancer, a cancer including MUC1 expressing cancer cells, or a cancer including TARP expressing cancer cells.

As used herein, a “HPV-related cancer” refers to any cancer that is resulting from an infection with an HPV. Non-limiting examples of HPV-related cancer include anal cancer, cervical cancer, oropharyngeal cancer, penile cancer, vaginal cancer, and vulvar cancer.

As used herein, a “cancer including MUC1 expressing cancer cells,” or a “cancer including TARP expressing cancer cells” is meant to refer to any type of cancer, with the condition that cells from said cancer (regardless of the percent of cells) express MUC1 and/or TARP. Virtually any cancer type can include MUC1 and/or TARP expressing cancer cells. Non-limiting examples of cancer including MUC1 and/or TARP expressing cancer cells include head and neck cancer, prostate cancer, breast cancer and acute myeloid leukemia (AML).

In some aspects, the cancer is selected from the group consisting of vulvar cancer, vaginal cancer, cervical cancer, penile cancer, anal cancer, head and neck cancer, oropharyngeal cancer, prostate cancer, breast cancer, colon cancer, gall bladder cancer, non-small cell lung cancer or acute myeloid leukemia (AML).

In an additional embodiment, the invention provides a method of enhancing T cell infiltration and proliferation in a tumor microenvironment in a subject including administering to the subject a therapeutically effective amount of the pharmaceutical formulation including a fusion protein comprising an IL-12 peptide or a fragment thereof and an antibody or a fragment thereof described herein, or a therapeutically effective amount of any of the pharmaceutical compositions described herein, thereby enhancing T cell infiltration and proliferation.

In one aspect, the subject has cancer.

The methods described herein include the administration of a pharmaceutical formulation.

In various aspects, the pharmaceutical formulation includes: (a) from about 1 mM to 20 mM citric acid; (b) from about 50 mM to 200 mM L-arginine; (c) from about 10 mM to 200 mM sucrose; and (d) from about 0.01% (w/w) to 0.5% (w/w) PS20.

In some aspects, the pharmaceutical formulation includes: (a) about 10 mM citric acid, (b) about 100 mM L-arginine; (c) about 100 mM sucrose; and (d) about 0.05% (w/w) PS20.

### Combination Therapies

The methods described herein include the administration of the pharmaceutical composition described herein, which may be administered as part of a combination therapy with one or more additional therapies.

### (1) Anti-Cancer Treatment

In one aspect, the method further includes administering an anti-cancer treatment.

In some aspects, the anti-cancer treatment comprises a chemotherapeutic agent, an immunotherapeutic agent, or a targeted immunotherapeutic agent.

“Chemotherapeutic agents” and “antineoplastic agents” are well known cytotoxic agents, and include: (i) anti-microtubules agents comprising vinca alkaloids (vinblastine, vincristine, vinflunine, vindesine, and vinorelbine), taxanes (cabazitaxel, docetaxel, larotaxel, ortataxel, paclitaxel, and tesetaxel), epothilones (ixabepilone), and podophyllotoxin (etoposide and teniposide); (ii) antimetabolite agents comprising anti-folates (aminopterin, methotrexate, pemetrexed, pralatrexate, and raltitrexed), and deoxynucleoside analogues (azacitidine, capecitabine, carmofur, cladribine, clofarabine, cytarabine, decitabine, doxifluridine, floxuridine, fludarabine, fluorouracil, gemcitabine, hydroxycarbamide, mercaptopurine, nelarabine, pentostatin, tegafur, and thioguanine); (iii) topoisomerase inhibitors comprising Topoisomerase I inhibitors (belotecan, camptothecin, cositecan, gimatecan, exatecan, irinotecan, lurtotecan, silatecan, topotecan, and rubitecan) and Topoisomerase II inhibitors (aclarubicin, amrubicin, daunorubicin, doxorubicin, epirubicin, etoposide, idarubicin, merbarone, mitoxantrone, novobiocin, pirarubicin, teniposide, valrubicin, and zorubicin); (iv) alkylating agents comprising nitrogen mustards (bendamustine, busulfan, chlorambucil, cyclophosphamide, estramustine phosphate, ifosamide, mechlorethamine, melphalan, prednimustine, trofosfamide, and uramustine), nitrosoureas (carmustine (BCNU), fotemustine, lomustine (CCNU), N-Nitroso-N-methylurea (MNU), nimustine, ranimustine semustine (MeCCNU), and streptozotocin), platinum-based (cisplatin, carboplatin, dicycloplatin, nedaplatin, oxaliplatin and satraplatin), aziridines (carboquone, thiotepa, mytomycin, diaziquone (AZQ), triaziquone and triethylenemelamine), alkyl sulfonates (busulfan, mannosulfan, and treosulfan), non-classical alkylating agents (hydrazines, procarbazine, triazenes, hexamethylmelamine, altretamine, mitobronitol, and pipobroman), tetrazines (dacarbazine, mitozolomide and temozolomide); (v) anthracyclines agents comprising doxorubicin and daunorubicin. Derivatives of these compounds include epirubicin and idarubicin; pirarubicin, aclarubicin, and mitoxantrone, bleomycins, mitomycin C, mitoxantrone, and actinomycin; (vi) enzyme inhibitors agents comprising FI inhibitor (Tipifarnib), CDK inhibitors (Abemaciclib, Alvocidib, Palbociclib, Ribociclib, and Seliciclib), PrI inhibitor (Bortezomib, Carfilzomib, and Ixazomib), PhI inhibitor (Anagrelide), IMPDI inhibitor (Tiazofurin), LI inhibitor (Masoprocol), PARP inhibitor (Niraparib, Olaparib, Rucaparib), HDAC inhibitor (Belinostat, Panobinostat, Romidepsin, Vorinostat), and PIKI inhibitor (Idelalisib); (vii) receptor antagonist agent comprising ERA receptor antagonist (Atrasentan), Retinoid X receptor antagonist (Bexarotene), Sex steroid receptor antagonist (Testolactone); (viii) ungrouped agent comprising Amsacrine, Trabectedin, Retinoids (Alitretinoin Tretinoin) Arsenic trioxide, Asparagine depleters (Asparaginase/Pegaspargase), Celecoxib, Demecolcine Elesclomol, Elsamitrucin, Etoglucid, Lonidamine, Lucanthone, Mitoguazone, Mitotane, Oblimersen, Omacetaxine mepesuccinate, and Eribulin.

“Immunotherapeutic agents” refer to drugs that modulate the immune system for the treatment of cancer, autoimmune diseases, and other conditions. Immunotherapeutic agents include monoclonal antibodies, that bind to specific targets on cancer cells; immune checkpoint inhibitors, that block proteins that normally keep the immune system from overreacting; cytokines, that boost the immune system; fusion proteins, hybrid proteins created by combining the sequences of two different proteins; therapeutic immunogenic compositions, to boost the immune system's response to cancer cells; T-cell transfer therapy, to boost the ability of T cells to fight cancer; and immune system modulators, that enhance the body's immune response.

In many aspects, the immunotherapeutic agent includes an immune checkpoint inhibitor (ICI).

“Checkpoint inhibitor therapy” is a form of cancer treatment currently that uses immune checkpoints which affect immune system functioning. Immune checkpoints can be stimulatory or inhibitory. Tumors can use these checkpoints to protect themselves from immune system attacks. Checkpoint therapy can block inhibitory checkpoints, restoring immune system function. Checkpoint proteins include programmed cell death 1 protein (PDCD1, PD-1; also known as CD279) and its ligand, PD-1 ligand 1 (PD-L1, CD274), cytotoxic T-lymphocyte-associated protein 4 (CTLA-4), A2AR (Adenosine A2A receptor), B7-H3 (or CD276), B7-H4 (or VTCN1), BTLA (B and T Lymphocyte Attenuator, or CD272), IDO (Indoleamine 2,3-dioxygenase), KIR (Killer-cell Immunoglobulin-like Receptor), LAG3 (Lymphocyte Activation Gene-3), TIM-3 (T-cell Immunoglobulin domain and Mucin domain 3), and VISTA (V-domain Ig suppressor of T cell activation).

In various aspects, the ICI comprises a programmed cell death 1 protein (PD-1) inhibitor, a PD-1 ligand (PD-L1) inhibitor, a cytotoxic T-lymphocyte-associated protein 4 (CTLA-4) inhibitor, an adenosine A2A receptor (A2AR) inhibitor, a B7-H3 inhibitor, a B7-H4 inhibitor, a B and T Lymphocyte Attenuator (BTLA) inhibitor, an indoleamine 2,3-dioxygenase (IDO) inhibitor, a killer-cell immunoglobulin-like receptor (KIR), a lymphocyte activation gene-3 (LAG3) inhibitor, a T-cell immunoglobulin domain and mucin domain 3 (TIM-3) inhibitor, or a V-domain Ig suppressor of T cell activation (VISTA) inhibitor.

Programmed cell death protein 1, also known as PD-1 and CD279 (cluster of differentiation 279), is a cell surface receptor that plays an important role in down-regulating the immune system and promoting self-tolerance by suppressing T cell inflammatory activity. PD-1 is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells).

PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney. PD-L1 is expressed on almost all murine tumor cell lines, including PAI myeloma, P815 mastocytoma, and B16 melanoma upon treatment with IFN-γ. PD-L2 expression is more restricted and is expressed mainly by DCs and a few tumor lines.

CTLA4 or CTLA-4 (cytotoxic T-lymphocyte-associated protein 4), also known as CD152 (cluster of differentiation 152), is a protein receptor that, functioning as an immune checkpoint, downregulates immune responses. CTLA4 is constitutively expressed in regulatory T cells but only upregulated in conventional T cells after activation—a phenomenon which is particularly notable in cancer. CTLA4 is a member of the immunoglobulin superfamily that is expressed by activated T cells and transmits an inhibitory signal to T cells. CTLA4 is homologous to the T-cell co-stimulatory protein, CD28, and both molecules bind to CD80 and CD86, also called B7-1 and B7-2 respectively, on antigen-presenting cells. CTLA4 binds CD80 and CD86 with greater affinity and avidity than CD28 thus enabling it to outcompete CD28 for its ligands. CTLA4 transmits an inhibitory signal to T cells, whereas CD28 transmits a stimulatory signal. CTLA4 is also found in regulatory T cells and contributes to its inhibitory function. T cell activation through the T cell receptor and CD28 leads to increased expression of CTLA4.

In one aspect, the ICI includes a PD-1 or a PD-L1 inhibitor.

In another aspect, the ICI includes pembrolizumab, nivolumab, cemiplimab, dostarlimab, retifanlimab, toripalimab, atezolizumab, avelumab, durvalumab, ipilimumab, lirilumab, tremelimumab, cosibelimab or relatlimab.

In many aspects, the ICI includes pembrolizumab.

### (2) Immune Modulators

In another aspect, the method further includes administering an immune modulator, wherein the immune modulator includes a peptide and a cationic lipid.

As used herein, the term “immune modulator” or “immune system modulator” refers to an agent that enhances the body's immune response. Non-limiting examples of immune modulators include peptides, which as described herein are associated with an immunogenic enhancer such as a cationic lipid.

### Peptides

The terms “peptide”, “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

As used herein, the terms “polyepitope peptide”, “multiepitope peptide” and the like refer to peptide or polypeptide that includes at least two epitopes as described herein. For example, the polyepitope peptide includes 2, 3, 4, 5, 6, 7, 8, 9, 10 or more of the epitopes of the invention.

The term “epitope” refers to an antigenic determinant in a molecule such as an antigen, i.e., to a part in or fragment of the molecule that is recognized by the immune system. An epitope of a protein such as a tumor antigen preferably comprises a continuous or discontinuous portion of said protein. The terms “epitope”, “antigen peptide”, “antigen epitope”, “immunogenic peptide”, “antigenic fragment” and “MHC binding peptide” can be used interchangeably herein and preferably relate to a representation of an antigen which is capable of eliciting an immune response against the antigen or a cell expressing or comprising and preferably presenting the antigen. An “antigen” according to the invention covers any substance that will elicit an immune response. In particular, an “antigen” relates to any substance, preferably a peptide or protein, that reacts specifically with antibodies or T-lymphocytes (T cells). According to the present invention, the term “antigen” comprises any molecule which comprises at least one epitope. Preferably, an antigen in the context of the present invention is a molecule which, optionally after processing, induces an immune reaction. According to the present invention, any suitable antigen may be used, which is a candidate for an immune reaction, wherein the immune reaction is preferably a cellular immune reaction. In the context of the embodiments of the present invention, the antigen is preferably presented by a cell, preferably by an antigen presenting cell which includes a diseased cell, in particular a cancer cell, in the context of MHC molecules, which results in an immune reaction against the antigen. An antigen is preferably a product which corresponds to or is derived from a naturally occurring antigen. Such naturally occurring antigens include tumor antigens.

A multiepitope peptide of the present invention, can include at least one multiepitope peptide, at least two multiepitope peptides, at least three multiepitope peptides, at least four multiepitope peptides, at least five multiepitope peptides, at least six multiepitope peptides, at least seven multiepitope peptides, at least eight multiepitope peptides, at least nine multiepitope peptides and at least ten multiepitope peptides. In one aspect, the multiepitope peptide includes at least two multiepitope peptides. A multiepitope peptide of the present invention, can include one multiepitope peptide, two multiepitope peptides, three multiepitope peptides, four multiepitope peptides, five multiepitope peptides, six multiepitope peptides, seven multiepitope peptides, eight multiepitope peptides, nine multiepitope peptides and ten multiepitope peptides. In one aspect, the multiepitope peptide includes two multiepitope peptides.

The multiepitope peptides of the present invention may be modified. In one aspect, the at least one multiepitope peptide is covalently modified. In one aspect, the modification includes palmitoylation or addition of an anionic sequence. In one aspect, the anionic sequence is SSEEEDE (SEQ ID NO:1). In one aspect, the anionic sequence is SSEEEDEE (SEQ ID NO:2). In one aspect, the anionic sequence is SEEEDESS (SEQ ID NO:3). In one aspect, the anionic sequence is SEEEDESEED (SEQ ID NO:4).

The term “long peptide” as used herein refers to a peptide of at least 30 amino acids. The long peptides of the present invention have immunological properties not found in shorter peptides or in pools of shorter peptides that cover the same sequence.

In one aspect, the at least one peptide has an amino acid sequence with at least 80%, 85%, 90% or 95% sequence identity with any one of the amino acid sequences provided herein.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity-number of identical positions/total number of positions (i.e., overlapping positions)×100). In some embodiments the length of a reference sequence (e.g., any of SEQ ID NOs: 1-49) aligned for comparison purposes is at least 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%. Percent identities between a disclosed sequence and a claimed sequence can be at least 85%. Percent identities between a disclosed sequence and a claimed sequence can be at least 90%. Percent identities between a disclosed sequence and a claimed sequence can be at least 95%. Percent identities between a disclosed sequence and a claimed sequence can be at least 96%. Percent identities between a disclosed sequence and a claimed sequence can be at least 97%. Percent identities between a disclosed sequence and a claimed sequence can be at least 98%. Percent identities between a disclosed sequence and a claimed sequence can be at least 99%. Percent identities between a disclosed sequence and a claimed sequence can be at least 99.5%. Percent identities between a disclosed sequence and a claimed sequence can be at least 99.9%. In general, an exact match indicates 100% identity over the length of the reference sequence (e.g., any of SEQ ID NOs: 1-49).

Polypeptides and polynucleotides that are about 80, 81, 82, 83, 84, 85, 86, 87, 88, 89, 90, 91, 92, 93, 94, 95, 96, 97, 98, 99, 99.5% or more identical to polypeptides and polynucleotides described herein are embodied within the disclosure. For example, a polypeptide can have 80%, 85%, 90%, 91%, 92%, 93%, 94%, 95%, 96%, 97%, 98%, 99% or more identity to SEQ ID NOs: 5- 49. In some aspects, a polypeptide can have 80% identity to any of SEQ ID NOs: 5-49. In some aspects, a polypeptide can have 85% identity to any of SEQ ID NOs: 5-49. In some aspects, a polypeptide can have 90% identity to any of SEQ ID NOs: 5-49. In some aspects, a polypeptide can have 91% identity to any of SEQ ID NOs: 5-49. In some aspects, a polypeptide can have 92% identity to any of SEQ ID NOs: 5-49. In some aspects, a polypeptide can have 93% identity to any of SEQ ID NOs: 5-49. In some aspects, a polypeptide can have 94% identity to any of SEQ ID NOs: 5-49. In some aspects, a polypeptide can have 95% identity to any of SEQ ID NOs: 5-49. In some aspects, a polypeptide can have 96% identity to any of SEQ ID NOs: 5-49. In some aspects, a polypeptide can have 97% identity to any of SEQ ID NOs: 5-49. In some aspects, a polypeptide can have 98% identity to any of SEQ ID NOs: 5-49. In some aspects, a polypeptide can have 99% identity to any of SEQ ID NOs: 5-49. In some aspects, a polypeptide can have 99.5% identity to any of SEQ ID NOs: 5-49. In some aspects, a polypeptide can have the amino acid sequence of any of SEQ ID NOs: 5-49.

Variants of the disclosed sequences also include peptides, or full-length protein, that contain substitutions, deletions, or insertions into the protein backbone, that would still leave at least about 70% homology to the original protein over the corresponding portion. A yet greater degree of departure from homology is allowed if like-amino acids, i.e., conservative amino acid substitutions, do not count as a change in the sequence. Examples of conservative substitutions involve amino acids that have the same or similar properties. Illustrative amino acid conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

In some aspects, the peptide includes at least one HPV peptide, at least one mucin 1 (MUC1) peptide, or at least one T-cell receptor alternate reading frame (TARP) peptide.

The epitopes referred to in the present application include any epitope that can be derived from an HPV peptide, a TARP peptide or a MUC1 peptide.

In various aspects, the at least one HPV peptide includes multiepitope HPV peptides. In some aspects, the at least one HPV peptide includes HPV peptides having a sequence having at least 80%, 85%, 90% or 95% identity with the sequence of any of SEQ ID NOs: 5-12.

In some aspects, the at least one HPV peptide includes HPV16 E6 peptides, and/or HPV16 E7.

HPV E6 and E7 proteins are constitutively co-expressed in all HPV infected precancerous cells and are the most abundant viral transcripts found in biopsies from HPV-related cervical carcinoma cells. Because of then-interaction with the p53 and retinoblastoma proteins, E6 and E7 are responsible for the transformation of cells and are required for the maintenance of HPV-associated malignancies. Notably, E6- and E7-specific cellular immune responses are associated with regression of HPV16-associated lesions. Compared to women with persistent cervical HPV16 infection, the percentages of positive enzyme-linked immunospot (ELISpot) responses to HPV16 E6 and E7 are significantly increased among women with recently resolved HPV infection. Therefore, the HPV E6 and E7 antigens are promising immunotherapeutic targets. To date, several types of HPV therapeutic immunogenic compositions, including protein/peptide-based immunogenic compositions, have been developed with a focus on stimulating the production and activation of HPV E6 and E7-specific T cells. However, these peptide-based HPV immunogenic compositions due to restricted HLA-A2 epitopes demonstrated very limited applicability even within the selected HLA-A2 populations evaluated. It was reported in 2009 that an HPV peptide immunogenic composition containing 13 overlapping peptides from the HPV16 E6 protein and 4 overlapping peptides from the HPV16 E7 peptide, a total of 13 peptides demonstrated robust anti-HPV response when studied in a non-restricted patient population with VIN3. This study provided the first demonstration of a broadly acting HPV peptide immunogenic compositions in a non-HLA-restricted population

Provided herein are HPV peptide sequences comprising 2-8 peptide sequences, 2-6 peptide sequences, or 4 peptide sequences. An important consideration in the design of these peptide-based immunogenic compositions designed to elicit CD8⁺ T cell generation and response in humans, was the polymorphism of the HLA class I molecules in the population. Because different HLA alleles bind different peptides, it is important that a peptide immunogenic composition contains enough different peptides to be immunogenic in a high percentage of the population. The HPV multiepitope peptide covers immunogenic regions of the HPV16 E6 and E7 proteins to provide correct processing and presentation of CD8⁺ T-cell epitopes in humans: in an embodiment, the immunogenic composition comprises four HPV-related peptides selected based on their binding and immunogenic activity.

Examples of HPV peptides include GQAEPDRAHYNIVTF (SEQ ID NO: 5), MHGDTPTLHEYMLDLQPETT (SEQ ID NO:6), LLMGTLGIVCPICSQKP (SEQ ID NO:7), and ELQTTIHDIILECVYCKQQLL (SEQ ID NO:8), or their lipidated versions.

The peptides may be present in the composition as individual peptides or they may be conjugated to each other (in any order), either with a spacer or without a spacer, to form a single long peptide encompassing the claimed sequences in accordance with methods known to those skilled in the art.

Non-limiting examples of HPV long peptides include peptides including SEQ ID NO:5, SEQ ID NO:6, SEQ ID NO:7 and SEQ ID NO:8; or SEQ ID NO:9, SEQ ID NO: 10, SEQ ID NO: 11 and SEQ ID NO:12.

In some aspects, the at least one MUC1 peptide includes MUC1 peptides have a sequence having at least 80%, 85%, 90% or 95% identity with the sequence of any of SEQ ID NOs: 13-37.

Mucin 1 (also known as CD227, episialin, PEM, H23Ag, EMA, CA15-3, and MCA) is a transmembrane glycoprotein aberrantly expressed on many epithelial cancer cells and varies in its cellular distribution, function, and glycosylation. The protein is a heterodimer with a large extracellular domain covalently bound to a small intracellular domain. The extracellular domain consists of a large number of tandem repeat regions (VNTR) and non-tandem regions. The C-terminus domain of MUC1 consists of interaction sites for several signaling molecules and has been shown to have oncogenic potential. Human clinical trials testing Mucin 1 (MUC1) as a tumor associated antigen therapeutic target have used several approaches to generate cytotoxic T cells capable of killing MUC1 expressing cancer cells. These clinical trials included the use of MUC1 polypeptides, DNA sequences or viral vectors consisting of MUC1 N-terminus immunogenic VNTR region and non-VNTR region derived T cell epitopes to generate cytotoxic T-cells. None of these approaches were able to meet clinical benefit criteria due to their inability to generate sufficient T cells capable of killing MUC1 expressing cancer cells. Current approaches target sequences from the C-Terminus of MUC1 and peptide modifications to generate epitope enhanced polypeptides that can improve immunogenicity of the composition.

Examples of MUC1 peptides include

(SEQ ID NO: 13)
YLAIVYLIALAVCQVRRKNYGQLDIFPARDKYHPMSEYAL,
(SEQ ID NO: 14)
YLAIVYLIAL,
(SEQ ID NO: 15)
YLIALAVCQV,
(SEQ ID NO: 16)
ALWGQDVTSV,
(SEQ ID NO: 17)
YLSYTNPAV,
(SEQ ID NO: 18)
YLAPPAHGV,
(SEQ ID NO: 19)
YLDTRPAPV,
(SEQ ID NO: 20)
YLAIVTLIAL,
(SEQ ID NO: 21)
YLIALAVCQV,
(SEQ ID NO: 22)
YLAPPAHGV,
(SEQ ID NO: 23)
YLDTRPAPV,
(SEQ ID NO: 24)
YLSYTNPAV,
(SEQ ID NO: 25)
ALFIVYLIAK,
(SEQ ID NO: 26)
SLFRSPYEK,
(SEQ ID NO: 27)
KYHPMSEYAL,
(SEQ ID NO: 28)
KYTNPAVAL,
(SEQ ID NO: 29)
ALAIVYLIAL,
(SEQ ID NO: 30)
YLIALAVCQC,
(SEQ ID NO: 31)
STAPPAHGV,
(SEQ ID NO: 32)
APDTRPAPG,
(SEQ ID NO: 33)
SLSYTNPAV,
(SEQ ID NO: 34)
ALAIVYLIAL,
(SEQ ID NO: 35)
STDRSPYEK,
(SEQ ID NO: 36)
TYHPMSEYPT,
and
(SEQ ID NO: 37)
SYTNPAVAA.

The peptides may be present in the composition as individual peptides or they may be conjugated to each other (in any order), either with a spacer or without a spacer, to form a single long peptide encompassing the claimed sequences in accordance with methods known to those skilled in the art.

Non-limiting examples of MUC1 long peptides include peptides including SEQ ID NO: 14, SEQ ID NO:15, SEQ ID NO:18 and SEQ ID NO:19, such as a MUC1 long peptide including the amino acid sequence of SEQ ID NO: 13.

In some aspects, the at least one TARP peptide includes TARP peptides have a sequence having at least 80%, 85%, 90% or 95% identity with the sequence of any of SEQ ID NOs: 38-49.

The TARP (T-cell receptor alternate reading frame) protein is a 58 amino acid protein identified using an expressed sequence database. The mRNA for the protein is initiated in the Jy 1 exon of the T-cell receptor (TCR) γ sequence and the protein expressed is initiated in an alternative reading frame distinct from that of the TCR γ coding sequence. TARP is highly expressed in primary as well as metastatic prostate cancer and other cancers; and is expressed in both hormone sensitive and castrate resistant prostate cancer. TARP is expressed by both normal and malignant prostate tissue and is overexpressed in >90% of prostate cancer specimens prostate and ~50% of breast cancers, with very little or no expression in normal cells, making it a good target antigen for therapeutic vaccination. Engineered CD8⁺ T cells recognizing the TARP derived T cell epitope can kill, in an antigen-dependent manner, tumor cells expressing TARP proteins.

Examples of TARP peptides include FVFLRNFSL (SEQ ID NO:38), FLRNFSLMV (SEQ NO: 39), ID MQMFPPSPLFFFLQLLKQSS (SEQ ID NO:40), FFLQLLKQSSRRLEHTFVFL (SEQ ID NO:41), RRLEHTFVFLRNFSLMLLRG (SEQ ID NO: 42), RNFSLMLLRGIGKKRRATRF (SEQ ID NO:43), and IGKKRRATRFWDPRRGTP (SEQ ID NO:44).

The peptides may be present in the composition as individual peptides or they may be conjugated to each other (in any order), either with a spacer or without a spacer, to form a single long peptide encompassing the claimed sequences in accordance with methods known to those skilled in the art.

Non-limiting examples of TARP long peptides include long peptides including MQMFPPSPLFFFLQLLKQSSRRLEHTFVFLRNFSLMVL (SEQ ID NO:45) and FVFLRNFSLMVLRGIGKKRRATRFWDPRRGTP (SEQ ID NO:46), and their anionic tag variants SEEEDESSMQMFPPSPLFFFLQLLKQSSRRLEHTFVFLRNFSLMV (SEQ ID NO: 47), SEEEDESEEDFVFLRNFSLMVLRGIGKKRRATRFWDPRRGTP (SEQ ID NO:48) and SEEEDESSMQMFPPSPLFFFLQLLKQSSRRLEHTFVFLRNFSLMLV (SEQ ID NO:49).

In one aspect, the at least one HPV peptide includes HPV16 E6 peptides, and/or HPV16 E7 peptides. In some aspects, the at least one HPV peptide includes an amino acid sequence of any of SEQ ID NOs: 5-12. In other aspects, the at least one MUC1 peptide includes an amino acid sequence of any of SEQ ID NOs: 13-37. In some aspects, the at least one TARP peptide includes an amino acid sequence of any of SEQ ID NOs: 38-49. In various aspects, the peptide comprises an animo acid sequence of any of SEQ ID NOs: 1-53.

### Immunogenic Enhancer—Cationic Lipid

In an embodiment, the immunogenic enhancer of the composition consists of a cationic lipid.

Cationic lipids have been reported to have strong immune-stimulatory/immunogenic enhancer effect. The cationic lipids of the present invention may form liposomes that are optionally mixed with antigen and may contain the cationic lipids alone or in combination with neutral lipids. Suitable cationic lipid species include: 3-β[N—N, 8-diguanidino spermidine)-carbamoyl] cholesterol (BGSC); 3-[N,N-diguanidinoethyl-aminoethane)-carbamoyl]cholesterol (BGTC); N,N1N2N3Tetra-methyltetrapalmitylspermine (cellfectin); N-t-butyl-N′-tetradecyl-3-tetradecyl-aminopropion-amidine (CLONfectin); dimethyldioctadecyl ammonium bromide (DDAB); 1,2-dimyristyloxypropyl-3-dimethyl-hydroxy ethyl ammonium bromide (DMRIE); 2,3-dioleoyloxy-N-[2 (sperminecarboxamido)ethyl]-N,N-dimethyl-1-p-ropanaminium trifluorocetate) (DOSPA); 1,3-dioleoyloxy-2-(6-carboxyspermyl)-propyl amide (DOSPER); 4-(2,3-bis-palmitoyloxy-propyl)-1-methyl-1H-imidazole (DPIM) N,N,N′,N′-tetramethyl-N,N′-bis(2-hydroxyethyl)-2,3 dioleoyloxy-1,4-butanediammonium iodide) (Tfx-50); N-1-(2,3-dioleoyloxy) propyl-N,N,N-trimethyl ammonium chloride (DOTMA) or other N—(N,N-1-dialkoxy)-alkyl-N,N,N-trisubstituted ammonium surfactants; 1,2 dioleoyl-3-(4′-trimethylammonio) butanol-sn-glycerol (DOBT) or cholesteryl (4′trimethylammonia) butanoate (ChOTB) where the trimethylammonium group is connected via a butanol spacer arm to either the double chain (for DOTB) or cholesteryl group (for ChOTB); DORI (DL-1,2-dioleoyl-3-dimethylaminopropyl- -hydroxyethylammonium) or DORIE (DL-1,2-O-dioleoyl-3-dimethylaminopropyl- -hydroxyethylammoniu-m) (DORIE) or analogs thereof as disclosed in WO 93/03709; 1,2-dioleoyl-3-succinyl-sn-glycerol choline ester (DOSC); cholesteryl hemisuccinate ester (ChOSC); lipopolyamines such as dioctadecylamidoglycylspermine (DOGS) and dipalmitoyl phosphatidylethanolamylspermine (DPPES) or the cationic lipids disclosed in U.S. Pat. No. 5,283,185, cholesteryl-3-carboxyl-amido-ethylenetrimethylammonium iodide, 1-dimethylamino-3-trimethylammonio-DL-2-propyl-cholesteryl carboxylate iodide, cholesteryl-3-O-carboxyamidoethyleneamine, cholesteryl-3- -oxysuccinamido-ethylenetrimethylammonium iodide, 1-dimethylamino-3-trimethylammonio-DL-2-propyl-cholesteryl-3″-oxysuccinate iodide, 2-(2-trimethylammonio)-ethylmethylamino ethyl-cholesteryl-3″-oxysuccinate iodide, 3- —N—(N′,N′-dimethylaminoethane) carbamoyl cholesterol (DC-chol), and 3- —N-(polyethyleneimine)-carbamoylcholesterol; O,O′-dimyristyl-N-lysyl aspartate (DMKE); O,O′-dimyristyl-N-lysyl-glutamate (DMKD); 1,2-dimyristyloxypropyl-3-dimethyl-hydroxy ethyl ammonium bromide (DMRIE); 1,2-dilauroyl-sn-glycero-3-ethylphosphocholine (DLEPC); 1,2-dimyristoyl-sn-glycero-3-ethylphosphocholine (DMEPC); 1,2-dioleoyl-sn-glycero-3-ethylphosphocholine (DOEPC); 1,2-dipalmitoyl-sn-glycero-3-ethylphosphocholine (DPEPC); 1,2-distearoyl-sn-glycero-3-ethylphosphocholine (DSEPC); 1,2-dioleoyl-3-trimethylammoninum propane (DOTAP); dioleoyl dimethylaminopropane (DODAP); 1,2-palmitoyl-3-trimethylammonium propane (DPTAP); 1,2-distearoyl-3-trimethylammonium propane (DSTAP), 1,2-myristoyl-3-trimethylammonium propane (DMTAP); and sodium dodecyl sulfate (SDS). The present invention contemplates the use of structural variants and derivatives of the cationic lipids disclosed in this application.

In one embodiment, chiral cationic lipids are lipids in which bonds between the lipophilic group and the amino group are stable in aqueous solution. Thus, an attribute of the complexes of the invention is their stability during storage (i.e., their ability to maintain a small diameter and retain biological activity over time following their formation). Such bonds used in the cationic lipids include amide bonds, ester bonds, ether bonds and carbamoyl bonds. Those of skill in the art would readily understand that liposomes containing more than one cationic lipid species may be used to produce the complexes of the present invention. For example, liposomes comprising two cationic lipid species, lysyl-phosphatidylethanolamine and β-alanyl cholesterol ester have been disclosed for certain drug delivery applications [Brunette, E. et al., Nucl. Acids Res., 20:1151 (1992)].

It is to be further understood that in considering chiral cationic liposomes suitable for use described herein and optionally mixing with antigen, the methods described herein are not restricted only to the use of the cationic lipids recited above but rather, any lipid composition may be used so long as a cationic liposome is produced and the resulting cationic charge density is sufficient to activate and induce an immune response.

Thus, the lipids described herein may contain other lipids in addition to the cationic lipids. These lipids include, but are not limited to, lyso lipids of which lysophosphatidylcholine (1-oleoyl lysophosphatidylcholine) is an example, cholesterol, or neutral phospholipids including dioleoyl phosphatidyl ethanolamine (DOPE) or dioleoyl phosphatidylcholine (DOPC) as well as various lipophylic surfactants, containing polyethylene glycol moieties, of which Tween-80 and PEG-PE are examples.

The cationic lipids described herein may also contain negatively charged lipids as well as cationic lipids so long as the net charge of the complexes formed is positive and/or the surface of the complex is positively charged. Negatively charged lipids of the invention are those comprising at least one lipid species having a net negative charge at or near physiological pH or combinations of these. Suitable negatively charged lipid species include, but are not limited to, CHEMS (cholesteryl hemisuccinate), NGPE (N-glutaryl phosphatidlylethanolanine), phosphatidyl glycerol and phosphatidic acid or a similar phospholipid analog.

Methods for producing the liposomes to be used in the production of the lipid comprising drug delivery complexes of the present invention are known to those of ordinary skill in the art. A review of methodologies of liposome preparation may be found in Liposome Technology (CFC Press New York 1984); Liposomes by Ostro (Marcel Dekker, 1987); Methods Biochem Anal. 33:337-462 (1988) and U.S. Pat. No. 5,283,185. Such methods include freeze-thaw extrusion and sonication. Both unilamellar liposomes (less than about 200 nm in average diameter) and multilamellar liposomes (greater than about 300 nm in average diameter) may be used as starting components to produce the complexes of this invention.

In the cationic liposomes utilized to produce the compositions described herein, the cationic lipid is present in the liposome at from about 10 mole % to about 100 mole % of total liposomal lipid, or from about 20 mole % to about 80 mole %. The neutral lipid, when included in the liposome, may be present at a concentration from about 0 mole % to about 90 mole % of the total liposomal lipid, or from about 20 mole % to about 80 mole %, or from 40 mole % to 80 mole %. The negatively charged lipid, when included in the liposome, may be present at a concentration ranging from about 0 mole % to about 49 mole % of the total liposomal lipid, or from about 0 mole % to about 40 mole %. In one embodiment, the liposomes contain a cationic and a neutral lipid, in ratios between about 2:8 to about 6:4. It is further understood that the complexes of the present invention may contain modified lipids, protein, polycations or receptor ligands which function as a targeting factor directing the complex to a particular tissue or cell type. Examples of targeting factors include, but are not limited to, asialoglycoprotein, insulin, low density lipoprotein (LDL), folate and monoclonal and polyclonal antibodies directed against cell surface molecules. Furthermore, to modify the circulatory half-life of the complexes, the positive surface charge can be sterically shielded by incorporating lipophilic surfactants which contain polyethylene glycol moieties.

The cationic lipid immunogenic compositions may be stored in isotonic sucrose or dextrose solution upon collection from the sucrose gradient or they may be lyophilized and then reconstituted in an isotonic solution prior to use. In one embodiment, the cationic lipid complexes are stored in solution. The stability of the cationic lipid complexes of the present invention is measured by specific assays to determine the physical stability and biological activity of the cationic lipid immunogenic compositions over time in storage. The physical stability of the cationic lipid immunogenic compositions is measured by determining the diameter and charge of the cationic lipid complexes by methods known to those of ordinary skill in the art, including for example, electron microscopy, gel filtration chromatography or by means of quasi-elastic light scattering using, for example, a Coulter N4SD particle size analyzer as described in the examples. The physical stability of the cationic lipid complex is “substantially unchanged” over storage when the diameter of the stored cationic lipid immunogenic compositions is not increased by more than 100%, or by not more than 50%, or by not more than 30%, over the diameter of the cationic lipid complexes as determined at the time the cationic lipid immunogenic compositions were purified.

While it is possible for the cationic lipid to be administered in a pure or substantially pure form, it is preferable to present it as a pharmaceutical composition, formulation or preparation. Pharmaceutical formulations using the chiral cationic lipid complexes of the invention may comprise the cationic lipid immunogenic compositions in a physiologically compatible sterile buffer such as, for example, phosphate buffered saline, isotonic saline or low ionic strength buffer such as acetate or Hepes (an exemplary pH being in the range of about 5.0 to about 8.0). The chiral cationic lipid immunogenic compositions may be administered as aerosols or as liquid solutions for intratumoral, intraarterial, intravenous, intratracheal, intraperitoneal, subcutaneous, and intramuscular administration.

The formulations of the present invention may incorporate any stabilizer known in the art. Illustrative stabilizers are cholesterol and other sterols that may help rigidify the liposome bilayer and prevent disintegration or destabilization of the bilayer. Also, agents such as polyethylene glycol, poly-, and mono-saccharides may be incorporated into the liposome to modify the liposome surface and prevent it from being destabilized due to interaction with blood-components. Other illustrative stabilizers are proteins, saccharides, inorganic acids, or organic acids which may be used either on their own or as admixtures.

A number of pharmaceutical methods may be employed to control, modify, or prolong the duration of immune stimulation. Controlled release preparations may be achieved through the use of polymer complexes such as polyesters, polyamino acids, methylcellulose, polyvinyl, poly(lactic acid), and hydrogels to encapsulate or entrap the cationic lipids and slowly release them. Similar polymers may also be used to adsorb the liposomes. The liposomes may be contained in emulsion formulations in order to alter the release profile of the stimulant. Alternatively, the duration of the stimulant's presence in the blood circulation may be enhanced by coating the surface of the liposome with compounds such as polyethylene glycol or other polymers and other substances such as saccharides which are capable of enhancing the circulation time or half-life of liposomes and emulsions.

When oral preparations are required, the chiral cationic lipids may be combined with typical pharmaceutical carriers known in the art such as, for example, sucrose, lactose, methylcellulose, carboxymethyl cellulose, or gum Arabic, among others. The cationic lipids may also be encapsulated in capsules or tablets for systemic delivery.

Administration of the chiral cationic lipid compositions of the present disclosure may be for either a prophylactic or therapeutic purpose. When provided prophylactically, the cationic lipid is provided in advance of any evidence or symptoms of illness. When provided therapeutically, the cationic lipid is provided at or after the onset of disease. The therapeutic administration of the immune-stimulant serves to attenuate or cure the disease. For both purposes, the cationic lipid may be administered with an additional therapeutic agent(s) or antigen(s). When the cationic lipids are administered with an additional therapeutic agent or antigen, the prophylactic or therapeutic effect may be generated against a specific disease.

In another aspect, the cationic lipid includes 1,2-dioleoyl-3-trimethylammonium propane (DOTAP), dimethyldioctadecyl ammonium (DDA), 1,2-dioleoyl-sn-glycero-3-ethylphosphocholine (DOEPC), N-1-(2,3-dioleoyloxy) propyl-N,N,N-trimethyl ammonium chloride (DOTMA), R-DOTAP, R-DDA, R-DOEPC, R-DOTMA, S-DOTAP, S-DDA, S-DOEPC, S-DOTMA, variations or analogs thereof.

In some aspects, the cationic lipid includes DOTAP.

In various aspects, the cationic lipid includes R-DOTAP.

In an embodiment, the compositions of the disclosure include peptides corresponding to SEQ ID NOs: 5-49, the immunogenic enhancer includes a cationic lipid, and the cationic lipid includes R-DOTAP.

In an embodiment, the compositions of the disclosure include peptides corresponding to any of SEQ ID NOs: 5-12, the immunogenic enhancer includes a cationic lipid, and the cationic lipid includes R-DOTAP.

In an embodiment, the compositions of the disclosure include peptides corresponding to SEQ ID NO:5, SEQ ID NO:6, SEQ ID NO:7 and SEQ ID NO:8, the immunogenic enhancer includes a cationic lipid, and the cationic lipid includes R-DOTAP.

In an embodiment, the compositions of the disclosure include peptides corresponding to SEQ ID NO:9, SEQ ID NO:10, SEQ ID NO:11 and SEQ ID NO: 12, the immunogenic enhancer includes a cationic lipid, and the cationic lipid includes R-DOTAP.

In an embodiment, the compositions of the disclosure include peptides corresponding to any of SEQ ID NOs: 13-37, the immunogenic enhancer includes a cationic lipid, and the cationic lipid includes R-DOTAP.

In an embodiment, the compositions of the disclosure include peptides corresponding to SEQ ID NO:14, SEQ ID NO: 15, SEQ ID NO: 18 and SEQ ID NO: 19, the immunogenic enhancer includes a cationic lipid, and the cationic lipid includes R-DOTAP.

In an embodiment, the compositions of the disclosure include peptides corresponding to SEQ ID NO:13, the immunogenic enhancer includes a cationic lipid, and the cationic lipid includes R-DOTAP.

In an embodiment, the compositions of the disclosure include peptides corresponding to any of SEQ ID NOs: 38-49, the immunogenic enhancer includes a cationic lipid, and the cationic lipid includes R-DOTAP.

In an embodiment, the compositions of the disclosure include peptides corresponding to any of SEQ ID NO:45, SEQ ID NO:46, SEQ ID NO:47, SEQ ID NO:48 and/or SEQ ID NO:49, the immunogenic enhancer includes a cationic lipid, and the cationic lipid includes R-DOTAP.

The above-described embodiments may be optionally encapsulated in liposomes. The above-described embodiments may be optionally combined with a pharmaceutically acceptable carriers and excipients e.g., various buffers such as acetate, phosphate and tonicity adjusters, such as sucrose, trehalose etc., or surfactants such as tween and others known to those skilled in the art.

In some embodiments the compositions may further comprise enhancer agonist epitopes such as the HBV core helper peptide etc., and or single-epitope peptides including, but not limited to YMLDLQPETT (SEQ ID NO:50), LLMGTLGIV (SEQ ID NO:51) or their lipidated versions.

The compositions described herein further comprise modified peptides, peptide analogs, and active fragments thereof. In certain embodiments the peptides may be modified by being oxidized, cross-linked by di-sulfide bonds, pegylated, glycosylated, phosphorylated, palmitoylated, methylated, biotinylated or by other processes known to those skilled in the art to improve efficacy and immunogenicity.

Sequences

| | |
|---|---|
| SSEEEDE | (SEQ ID NO: 1) |
| SSEEEDEE | (SEQ ID NO: 2) |
| SEEEDESS | (SEQ ID NO: 3) |
| SEEEDESEED | (SEQ ID NO: 4) |
| GQAEPDRAHYNIVTF | (SEQ ID NO: 5) |
| MHGDTPTLHEYMLDLQPETT | (SEQ ID NO: 6) |
| LLMGTLGIVCPICSQKP | (SEQ ID NO: 7) |
| ELQTTIHDIILECVYCKQQLL | (SEQ ID NO: 8) |
| KSS ELQTTIHDIILECVYCKQQLL | (SEQ ID NO: 9) |
| KSS MHGDTPTLHEYMLDLQPETT | (SEQ ID NO: 10) |
| KSS LLMGTLGIVCPICSQKP | (SEQ ID NO: 11) |
| KSS GQAEPDRAHYNIVTF | (SEQ ID NO: 12) |
| YLAIVYLIALAVCQVRRKNYGQLDIFPARDKYHPMSEYAL | (SEQ ID NO: 13) |
| YLAIVYLIAL | (SEQ ID NO: 14) |
| YLIALAVCQV | (SEQ ID NO: 15) |
| ALWGQDVTSV | (SEQ ID NO: 16) |
| YLSYTNPAV | (SEQ ID NO: 17) |
| YLAPPAHGV | (SEQ ID NO: 18) |
| YLDTRPAPV | (SEQ ID NO: 19) |
| YLAIVTLIAL | (SEQ ID NO: 20) |
| YLIALAVCQV | (SEQ ID NO: 21) |
| YLAPPAHGV | (SEQ ID NO: 22) |
| YLDTRPAPV | (SEQ ID NO: 23) |
| YLSYTNPAV | (SEQ ID NO: 24) |
| ALFIVYLIAK | (SEQ ID NO: 25) |
| SLFRSPYEK | (SEQ ID NO: 26) |
| KYHPMSEYAL | (SEQ ID NO: 27) |
| KYTNPAVAL | (SEQ ID NO: 28) |
| ALAIVYLIAL | (SEQ ID NO: 29) |
| YLIALAVCQC | (SEQ ID NO: 30) |
| STAPPAHGV | (SEQ ID NO: 31) |
| APDTRPAPG | (SEQ ID NO: 32) |
| SLSYTNPAV | (SEQ ID NO: 33) |
| ALAIVYLIAL | (SEQ ID NO: 34) |
| STDRSPYEK | (SEQ ID NO: 35) |
| TYHPMSEYPT | (SEQ ID NO: 36) |
| SYTNPAVAA | (SEQ ID NO: 37) |
| FVFLRNFSL | (SEQ ID NO: 38) |
| FLRNFSLMV | (SEQ ID NO: 39) |
| MQMFPPSPLFFFLQLLKQSS | (SEQ ID NO: 40) |
| FFLQLLKQSSRRLEHTFVFL | (SEQ ID NO: 41) |
| RRLEHTFVFLRNFSLMLLRG | (SEQ ID NO: 42) |
| RNFSLMLLRGIGKKRRATRF | (SEQ ID NO: 43) |
| IGKKRRATRFWDPRRGTP | (SEQ ID NO: 44) |
| MQMFPPSPLFFFLQLLKQSSRRLEHTFVFLRNFSLMVL | (SEQ ID NO: 45) |
| FVFLRNFSLMVLRGIGKKRRATRFWDPRRGTP | (SEQ ID NO: 46) |
| SEEEDESSMQMFPPSPLFFFLQLLKQSSRRLEHTFVFLRNFSLMV | (SEQ ID NO: 47) |
| SEEEDESEEDFVFLRNFSLMVLRGIGKKRRATRFWDPRRGTP | (SEQ ID NO: 48) |
| SEEEDESSMQMFPPSPLFFFLQLLKQSSRRLEHTFVFLRNFSLMLV | (SEQ ID NO: 49) |
| YMLDLQPETT | (SEQ ID NO: 50) |
| LLMGTLGIV | (SEQ ID NO: 51) |
| KSS YMLDLQPETT | (SEQ ID NO: 52) |
| KSS LLMGTLGIV | (SEQ ID NO: 53) |
| MELPVRLLVLMFWIPASLSSSELTQDPAVSVALGQTVRITCQG | (SEQ ID NO: 54) |
| DSLRSYYASWYQQKPGQAPVLVIYGKNNRPSGIPDRFSGSSS | |
| GNTASLTITGAQAEDEADYYCNSRDSSGNHVVFGGGTKVTV | |
| LGQPKAAPSVTLFPPSSEELQANKATLVCLISDFYPGAVTVAW | |
| KADSSPVKAGVETTTPSKQSNNKYAASSYLSLTPEQWKSHKS | |
| YSCQVTHEGSTVEKTVAPTECS | |
| SSSELTQDPAVSVALGQTVRITCQGDSLRSYYASWYQQKPGQ | (SEQ ID NO: 55) |
| APVLVIYGKNNRPSGIPDRFSGSSSGNTASLTITGAQAEDEADY | |
| YCNSRDSSGNHVVFGGGTKVTVL | |
| MELPVRLLVLMFWIPASLSQVQLQESGPGLVKPSETLSLTCAVS | (SEQ ID NO: 56) |
| GYSISSGYYWGWIRQPPGKGLEWIGSIYHSGSTYYNPSLKSRV | |
| TISVDTSKNQFSLKLSSVTAADTAVYYCARGKWSKFDYWGQG | |
| TLVTVSSASTKGPSVFPLAPSSKSTSGGTAALGCLVKDYFPEPVT | |
| VSWNSGALTSGVHTFPAVLQSSGLYSLSSVVTVPSSSLGTQTYIC | |
| NVNHKPSNTKVDKRVEPKSCDKTHTCPPCPAPELLGGPSVFLFP | |
| PKPKDTLMISRTPEVTCVVVDVSHEDPEVKFNWYVDGVEVHN | |
| AKTKPREEQYNSTYRVVSVLTVLHQDWLNGKEYKCKVSNKALP | |
| APIEKTISKAKGQPREPQVYTLPPSREEMTKNQVSLTCLVKGFYP | |
| SDIAVEWESNGQPENNYKTTPPVLDSDGSFFLYSKLTVDKSRWQ | |
| QGNVFSCSVMHEALHNHYTQKSATATPGAANLPVATPDPGMFPC | |
| LHHSQNLLRAVSNMLQKARQTLEFYPCTSEEIDHEDITKDKTSTV | |
| EACLPLELTKNESCLNSRETSFITNGSCLASRKTSFMMALCLSSIY | |
| EDLKMYQVEFKTMNAKLLMDPKRQIFLDQNMLAVIDELMQAL | |
| NFNSETVPQKSSLEEPDFYKTKIKLCILLHAFRIRAVTIDRVMSYL | |
| NAS | |
| QVQLQESGPGLVKPSETLSLTCAVSGYSISSGYYWGWIRQPPG | (SEQ ID NO: 57) |
| KGLEWIGSIYHSGSTYYNPSLKSRVTISVDTSKNQFSLKLSSV | |
| TAADTAVYYCARGKWSKFDYWGQGTLVTVSS | |
| MELPVRLLVLMFWIPASLS (signal peptide) | (SEQ ID NO: 58) |
| MCHQQLVISWFSLVFLASPLVAIWELKKDVYVVELDWYPDA | (SEQ ID NO: 59) |
| PGEMVVLTCDTPEEDGITWTLDQSSEVLGSGKTLTIQVKEFG | |
| DAGQYTCHKGGEVLSHSLLLLHKKEDGIWSTDILKDQKEPK | |
| NKTFLRCEAKNYSGRFTCWWLTTISTDLTFSVKSSRGSSDPQ | |
| GVTCGAATLSAERVRGDNKEYEYSVECQEDSACPAAEESLPI | |
| EVMVDAVHKLKYENYTSSFFIRDIIKPDPPKNLQLKPLKNSRQ | |
| VEVSWEYPDTWSTPHSYFSLTFCVQVQGKDNTEGRVFTDKT | |
| SATVICRKNASISVRAQDRYYSSSWSEWASVPCS | |
| KSKREKKDRV | (SEQ ID NO: 60) |
| KDNTEGRV | (SEQ ID NO: 61) |

### Kits

In a further embodiment, the invention provides a kit including (a) the pharmaceutical formulation including a fusion protein comprising an IL-12 peptide or a fragment thereof and an antibody or a fragment thereof described herein, or any of the pharmaceutical compositions described herein; and (b) instructions for use.

In one aspect, the kit further includes an anti-cancer treatment and/or an immune modulator.

Presented below are examples discussing the pharmaceutical formulation described herein, contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Objectives, Material and Methods

NHS-IL12, an antibody drug conjugate (ADC), is an antibody-cytokine recombinant fusion protein (IgG1 and IL-12) designed to target IL-12 to the phospho-diester backbone of DNA at areas of tumor necrosis, enhancing T cell infiltration and proliferation in the tumor microenvironment. Creating useful protein-based drug formulations is often challenging because of the instability arising from aggregation, unfolding, oxidation and other degradation tendencies of proteins under different conditions and stressors leading to decreases in their overall biological activity. To avoid issues caused by formulation instability, different strategies are often adopted in the development of therapeutic protein formulations. Among these strategies are mutations to the genome-encoding the protein, chemical changes to the protein itself, different purification methods adopted to maintain stability, selecting stability promoting buffers, pH and ionic strength modification, and/or the incorporations of various additives and excipients to prevent aggregation and/or unfolding or other degradation. Injectable therapeutic proteins need to be stable when stored and transferred at 2-8° C. and can be considered successful with a shelf life of 2 years to be economically viable. There is a very significant value to developing a liquid refrigerated protein drug formulation with long term stability, making product storage and transit efficient and applicable to a wide variety of treatment locations worldwide with no special handling conditions required. A refrigerated liquid formulation, ready to inject, also simplifies and improves clinical usage with no freezing and thawing or reconstitution of more costly freeze-dried product, thus minimizing potential errors in patient dosing and lowering product cost. However, due to inherent instability in liquid form, the majority of protein drugs are formulated and stored as freeze-fried powders.

Therefore, in efforts to produce an optimal and stable liquid IL-12 fusion protein formulation with a shelf life that is greater than two years (in refrigerated storage condition at about 2-8° C.), different combinations of buffers, surfactants, stabilizers, and sugars were studied to understand how the various interactions between the excipients and protein may enable long term refrigerated stability. Stability was evaluated and confirmed by characterizing the retention of compound structural stability and biological activity over time. Some of the more stable and commercially viable protein drugs stored as liquids include Humalog (24-month stability at 2- 8° C.), Kadcyla (48-month stability when stored as a freeze-dried powder and 24 hours at 2-8° C. once reconstituted into a liquid), Enbrel (30-month stability at 2-8° C.). For our studies, primary protein formulation stability determinations were made utilizing size exclusion high performance liquid chromatography (SE-HPLC) where monomer percent recovery was calculated and compared to the control/TO timepoint for the different stability points.

For these formulation studies, two different starting materials were tested. The first set of pilot studies were performed on formulated drug substances (DS) to determine initial stable buffer conditions using dialysis/buffer exchange techniques to confirm the optimal buffering conditions. The results guided the second set of experiments involving the unformulated raw material of IL-12 fusion protein (RM) at concentrations of 2.6 mg/mL. To determine and confirm the optimal formulation conditions, different buffering agents, stabilizers, surfactants and sugars/ionic strengths were tested. Under the different formulation conditions, IL-12 fusion protein accelerated stability was tested by holding the samples at 2-8° C. and 40° C. to assess the compound's stability over time. This was assessed by quantitating the monomer and when applicable the high molecular weight species, determining their percentage recoveries compared to the starting material. Protein concentration was also measured for all samples at the different stability timepoints. The collective results were used to confirm the optimal formulation conditions.

### Methods

For buffer exchange, dialysis was performed using the Slide-A-Lyzers 20K cassettes. These cassettes contain a large surface/volume ratio of regenerated cellulose membranes. The pilot studies were focused first on the buffer background, discounting any additives such as stabilizers and sugars. Based on the pilot stability studies in the plain buffer systems, appropriate modifications were applied to the buffers (addition of stabilizers and/or sugars) and the dialysis studies and stability timepoints were repeated.

Changing the dialysate multiple times removes the small molecules that are no longer in the sample and allows more to diffuse out into the dialysate. Post dialysis at 2-8° C., samples were aliquoted in glass vials, sealed, and stored at 2-8° C. and 40° C. for stability determination. TO as well as the different stability timepoints were prepared by diluting the samples to 0.4 mg/mL final concentration in 50 mM sodium acetate pH 6.0 (dilution buffer).

### Buffering Solution Preparations

The current formulation of the IL-12 fusion protein is 1.5 mg/mL protein and contains 10 mM citric acid, 100 mM L-arginine, 100 mM sucrose, 0.05% (w/w) polysorbate 20 (PS20). The final pH of the formulation is at pH 6.0.

A pH of 6.0 was chosen as it is close to one pH unit below the pI (isoelectric point) of IL-12 fusion protein (pI 6.73), while maintaining the pH as close as possible to physiological conditions to maximize biological activity and compatibility with patient comfort. Typically, where the pH of a formulation is one or more pH units above or below the pI of the protein, this helps reduce aggregation (isoelectric precipitation). Additionally, while neutral to mildly basic conditions favor deamidation, mildly acidic conditions markedly slow down the process, which is why pH 6.0 was selected.

The different buffering backgrounds tested were citric acid (citrate buffer), histidine, sodium phosphate, sodium acetate, and succinate. Once the optimal buffer was established, different stabilizers, sugars, and surfactants were tested using the optimal buffering background. For the stabilizers, the following were tested, arginine, methionine and niacinamide. For the sugars, sucrose, lactose, and mannitol were tested. Finally for surfactants the following were tested, polysorbate 20 (PS20), polysorbate 80 (PS80) and poloxamer 188 (P188). The following tables summarize the buffers preparations for the buffer exchange experiments (2-Liter Scale):

TABLE 1
Background Buffers Preparation

| | | | | Final | |
|---|---|---|---|---|---|
| | Reagents | Amount | | Conc. | |
| 10 mM | Milli-Q Water | 1800 | mL | NA | |
| Citrate | Citric Acid Monohydrate | 4.20 | g | 10 | mM |
| buffer, pH | (MW: 210.14 g/mol) | | | | |
| 6.0 ± 0.1 | NaOH 50% | — | | pH 6.0 ± 0.1 | |
| Procedure | In a graduated cylinder, transferred 1800 mL of water and added 4.20 g of citric | | | | |
| | acid. Stirred until completely dissolved. Adjusted pH to 6.0 ± 0.1 using 50% NaOH. | | | | |
| | Brought to final volume and filtered under vacuum conditions with 0.22 μm PES filter. | | | | |
| 10 mM | Milli-Q Water | 1800 | mL | NA | |
| Histidine | Histidine (MW: 209.63 g/mol) | 4.1926 | g | 10 | mM |
| buffer, | NaOH 50% or HCl as necessary | — | | pH 6.0 ± 0.1 | |
| pH 6.0 ± 0.1 | | | | | |
| Procedure | In a graduated cylinder, transferred 1800 mL of water and added 4.1926 g of | | | | |
| | histidine. Stirred until completely dissolved. Adjusted pH to 6.0 ± 0.1 using 50% NaOH or | | | | |
| | HCl as necessary. Brought to final volume and filtered under vacuum conditions with 0.22 | | | | |
| | μm PES filter. | | | | |
| 10 mM | Milli-Q Water | 1800 | mL | NA | |
| Sodium | NaH2PO4•H2O (MW: 137.99 g/mol) | 2.534 | g | 9.182 | mM |
| Phosphate, | Na2HPO4 (MW: 141.96 g/mol) | 0.232 | g | 0.818 | mM |
| pH 6.0 ± 0.1 | NaOH 50% or H3PO4 as necessary | — | | pH 6.0 ± 0.1 | |
| Procedure | In a graduated cylinder, added 2.534 g of sodium dihydrogen phosphate | | | | |
| | monohydrate (Na2HPO4•H2O), and 0.232 g of di-sodium hydrogen phosphate anhydrous | | | | |
| | (Na2HPO4) to 1800 mL of HPLC water. Stirred until the salts are completely dissolved. | | | | |
| | Adjusted pH to 6.0 ± 0.1 if necessary, using 50% NaOH. Brought to final volume and filtered | | | | |
| | under vacuum conditions with 0.22 μm PES filter. | | | | |
| 10 mM | Milli-Q Water | 1800 | mL | NA | |
| Sodium Acetate, | Acetic acid (MW: 60.05 g/mol) | 1.144 | mL | 10 | mM |
| pH 6.0 ± 0.1 | NaOH 50% | — | | pH 6.0 ± 0.1 | |
| Procedure | In a graduated cylinder, added 1.144 mL of acetic acid to 1800 mL of water and | | | | |
| | stirred to mix. Adjusted the pH to 6.0 ± 0.1 with 50% NaOH. Brought to final volume and | | | | |
| | filtered under vacuum conditions with 0.22 μm PES filter. | | | | |
| 10 mM | Milli-Q Water | 1800 | mL | NA | |
| Succinate buffer, | Succinic Acid Disodium Salt (MW: 162.05 g/mol) | 3.24 | g | 10 | mM |

| | | | |
|---|---|---|---|
| pH 6.0 ± 0.1 | HCl | — | pH 6.0 ± 0.1 |

| | |
|---|---|
| Procedure | In a graduated cylinder, transferred 1800 mL of water and added 3.24 g of succinic |
| | acid disodium salt and stirred until completely dissolved. Adjusted pH to 6.0 ± 0.1 using |
| | HCl. Brought to final volume and filtered under vacuum conditions with 0.22 μm PES filter. |

TABLE 2
Preparation of Citrate + Stabilizer Buffers

| | | | | Final | |
|---|---|---|---|---|---|
| | Reagents | Amount | | Conc. | |
| 10 mM | Milli-Q Water | 1800 | | — | |
| Citrate | Citric Acid Monohydrate (MW: 210.14 g/mol) | 4.20 | g | 10 | mM |
| buffer, 100 | L-Arginine (MW: 210.67 g/mol) | 42.134 | g | 100 | mM |
| mM Arginine, | NaOH | — | | pH 6.0 ± 0.1 | |
| pH 6.0 ± 0.1 | | | | | |
| Procedure | In a graduated cylinder, transferred 1800 mL of water, added 4.20 g of citric acid | | | | |
| | and 42.134 g of L-arginine. Stirred until completely dissolved. Adjusted pH to 6.0 ± 0.1 | | | | |
| | using 50% NaOH. Brought to final volume and filtered under vacuum conditions with 0.22 | | | | |
| | μm PES filter. | | | | |
| 10 mM | Milli-Q Water | 1800 | | — | |
| Citrate | Citric Acid Monohydrate (MW: 210.14 g/mol) | 4.20 | g | 10 | mM |
| buffer, 100 | L-Methionine (MW: 149.21 g/mol) | 29.842 | g | 100 | mM |
| mM Methionine, | NaOH | — | | pH 6.0 ± 0.1 | |
| pH 6.0 ± 0.1 | | | | | |
| Procedure | In a graduated cylinder, transferred 1800 mL of water, added 4.20 g of citric acid | | | | |
| | and 29.842 g of L-methionine. Stirred until completely dissolved. Adjusted pH to 6.0 ± 0.1 | | | | |
| | using 50% NaOH. Brought to final volume and filtered under vacuum conditions with 0.22 | | | | |
| | μm PES filter. | | | | |
| 10 mM | Milli-Q Water | 1800 | | — | |
| Citrate | Citric Acid Monohydrate (MW: 210.14 g/mol) | 4.20 | g | 10 | mM |
| buffer, 100 | Niacinamide (MW: 122.12 g/mol) | 24.42 | g | 100 | mM |
| mM Niacinamide, | NaOH | — | | pH 6.0 ± 0.1 | |
| pH 6.0 ± 0.1 | | | | | |

| | |
|---|---|
| Procedure | In a graduated cylinder, transferred 1800 mL of water, added 4.20 g of citric acid |
| | and 24.42 g of niacinamide. Stirred until completely dissolved. Adjusted pH to 6.0 ± 0.1 |
| | using 50% NaOH. Brought to final volume and filtered under vacuum conditions with 0.22 |
| | μm PES filter. |

TABLE 3
Preparation of Citrate + Sugar Buffers

| | | | | Final | |
|---|---|---|---|---|---|
| | Reagents | Amount | | Conc. | |
| 10 mM | Milli-Q Water | 1800 | | NA | |
| Citrate | Citric Acid Monohydrate (MW: 210.14 g/mol) | 4.20 | g | 10 | mM |
| buffer, 100 | Sucrose (MW: 342.30 g/mol) | 68.46 | g | 100 | mM |
| mM Sucrose, | NaOH | — | | pH 6.0 ± 0.1 | |
| pH 6.0 ± 0.1 | | | | | |
| Procedure | In a graduated cylinder, transferred 1800 mL of water, added 4.20 g of citric acid | | | | |
| | and 68.46 g of sucrose. Stirred until completely dissolved. Adjusted pH to 6.0 ± 0.1 using | | | | |
| | 50% NaOH or HCl. Brought to final volume and filtered under vacuum conditions with 0.22 | | | | |
| | μm PES filter. | | | | |
| 10 mM | Milli-Q Water | 1800 | | — | |
| Citrate buffer, | Citric Acid Monohydrate (MW: 210.14 g/mol) | 4.20 | g | 10 | mM |
| 100 mM Lactose, | Lactose (MW: 342.30 g/mol) | 68.46 | g | 100 | mM |
| pH 6.0 ± 0.1 | NaOH | — | | pH 6.0 ± 0.1 | |
| Procedure | In a graduated cylinder, transferred 1800 mL of water, added 4.20 g of citric acid | | | | |
| | and 68.46 g of lactose. Stirred until completely dissolved. Adjusted pH to 6.0 ± 0.1 using | | | | |
| | 50% NaOH or HCl. Brought to final volume and filtered under vacuum conditions with 0.22 | | | | |
| | μm PES filter. | | | | |
| 10 mM | Milli-Q Water | 1800 | | — | |
| Citrate | Citric Acid Monohydrate (MW: 210.14 g/mol) | 4.20 | g | 10 | mM |
| buffer, 100 | Mannitol (MW: 182.17 g/mol) | 36.434 | g | 100 | mM |
| mM Mannitol, | NaOH | — | | pH 6.0 ± 0.1 | |
| pH 6.0 ± 0.1 | | | | | |

| | |
|---|---|
| Procedure | In a graduated cylinder, transferred 1800 mL of water, added 4.20 g of citric acid |
| | and 36.434 g of mannitol. Stirred until completely dissolved. Adjusted pH to 6.0 ± 0.1 using |
| | 50% NaOH or HCl. Brought to final volume and filtered under vacuum conditions with 0.22 |
| | μm PES filter. |

TABLE 4
Preparation of Citrate + Surfactant Buffers

| | | | | Final | |
|---|---|---|---|---|---|
| | Reagents | Amount | | Conc. | |
| 10 mM | Milli-Q Water | 1800 | | — | |
| Citrate buffer, | Citric Acid Monohydrate (MW: 210.14 g/mol) | 4.20 | g | 10 | mM |
| 0.05% PS20 (w/v), | PS20 (density: 1.1 g/cm³) | 909 | μL | 0.05% | (w/v) |
| pH 6.0 ± 0.1 | NaOH | — | | pH 6.0 ± 0.1 | |
| Procedure | In a graduated cylinder, transferred 1800 mL of water, added 4.20 g of citric acid | | | | |
| | and 909 μL of PS20. Stirred until completely dissolved. Adjusted pH to 6.0 ± 0.1 using | | | | |
| | 50% NaOH. Brought to final volume and filtered under vacuum conditions with 0.22 μm | | | | |
| | PES filter. | | | | |
| 10 mM | Milli-Q Water | 1800 | | — | |
| Citrate | Citric Acid Monohydrate (MW: 210.14 g/mol) | 4.20 | g | 10 | mM |
| buffer, 0.05% | PS80 (density: 1.06 g/cm³) | 943 | μL | 0.05% | (w/v) |
| PS80 (w/v), | NaOH | — | | pH 6.0 ± 0.1 | |
| pH 6.0 ± 0.1 | | | | | |
| Procedure | In a graduated cylinder, transferred 1800 mL of water, added 4.20 g of citric acid | | | | |
| | and 943 μL of PS80. Stirred until completely dissolved. Adjusted pH to 6.0 ± 0.1 using | | | | |
| | 50% NaOH. Brought to final volume and filter under vacuum conditions with 0.22 μm | | | | |
| | PES filter. | | | | |
| 10 mM | Milli-Q Water | 1800 | | — | |
| Citrate | Citric Acid Monohydrate (MW: 210.14 g/mol) | 4.20 | g | 10 | mM |
| buffer, 0.05% | P188 | 0.5 | g | 0.05% | (w/v) |
| P188 (w/v), | NaOH | — | | pH 6.0 ± 0.1 | |
| pH 6.0 ± 0.1 | | | | | |

| | |
|---|---|
| Procedure | In a graduated cylinder, transferred 1800 mL of water, added 4.20 g of citric acid |
| | and 0.5 g of P188. Stirred until completely dissolved. Adjusted pH to 6.0 ± 0.1 using 50% |
| | NaOH. Brought to final volume and filtered under vacuum conditions with 0.22 μm PES |
| | filter. |

### Dialyzed Samples Stability

Dialyzed samples were removed from the dialysate and the exact volume of the samples were recorded with any volume increase. Samples' appearances were recorded including any sample precipitation. All samples, including un-dialyzed controls, were briefly centrifuged at 13,000 rcf for 5 minutes and the supernatants were processed for HPLC analysis. The samples were prepared for SE-HPLC analysis to record the baseline for all conditions at TO. The remaining sample aliquots (250 to 500 μL/vial) as well as control un-dialyzed controls were incubated at 2-8° C. and 40° C. and samples' stability was monitored at different time points.

The stability was determined by assessing the percent recoveries of the samples at different time points compared to their TO in terms of the monomer recovery. In addition to comparing samples to the TO, the controls like DS and RM were also run for reference. Additionally, system suitability was assessed for all sequences to ensure adequate system performance prior and during the samples' analyses.

### Example 2

### SE-HPLC Results

Protein concentrations of the dialyzed samples were determined at TO as well as the different timepoints utilizing the DS-11 FX Spectrophotometer.

### Results

System suitability was performed for each analytical sequence to ensure the system was properly functioning prior to and during sample analysis. FIG. 2 shows an example of the system suitability profiles.

As shown in FIG. 2, Control Sample (CS) of IL-12 fusion protein prepared at 0.4 mg/mL was run (n=6) for each analytical sequence to ensure adequate system performance prior and during sample analysis: % RSD of 0.56 and 0.01 for the monomer peak area and retention time, respectively.

### Example 3

### SE-HPLC Analysis—Using Drug Substance (DS)

The drug substance (DS) of the IL-12 fusion protein is formulated at 1.5 mg/mL protein and contains 10 mM citric acid monohydrate, 100 mM L-arginine monohydrate, 100 mM sucrose, 0.05% (w/w) polysorbate 20 (PS20). The final pH of the formulation is at pH 6.0. This material was essential to confirm the significance of citric acid buffering background on the formulation stability. The DS was also used to determine the optimal sugar and stabilizers because the current sucrose and arginine can be removed from the formulation via buffer exchange to allow for testing various sugars and stabilizers and analyze their effects on the current formulation stability. However, because the current formulation contains PS20 which cannot be removed during buffer exchange, surfactants effects were only tested later using the raw material (RM) only. The IL-12 fusion protein DS control was included in all SE-HPLC sequences as the positive control for reference when analyzing the different buffers, sugars, and stabilizers.

### Effect of Buffers on Formulation Stability

For the first set of experiments, the DS was used to determine the best buffering background for the formulation. The different buffers tested were citrate, histidine, sodium phosphate, sodium acetate and succinate at a concentration of 10 mM. The samples were analyzed immediately after dialysis as well as at 2 weeks after incubation at 5° C. and 40° C.

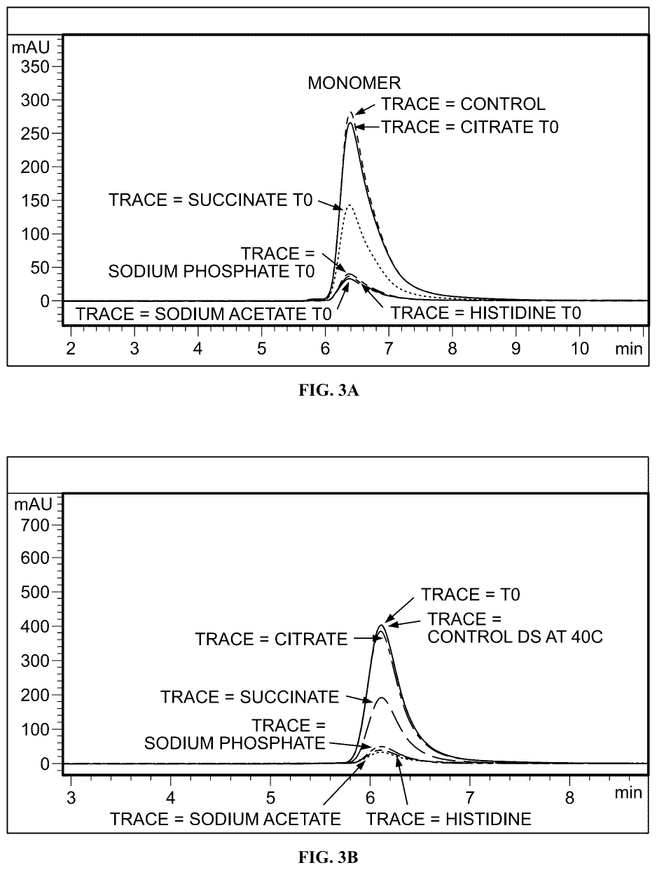

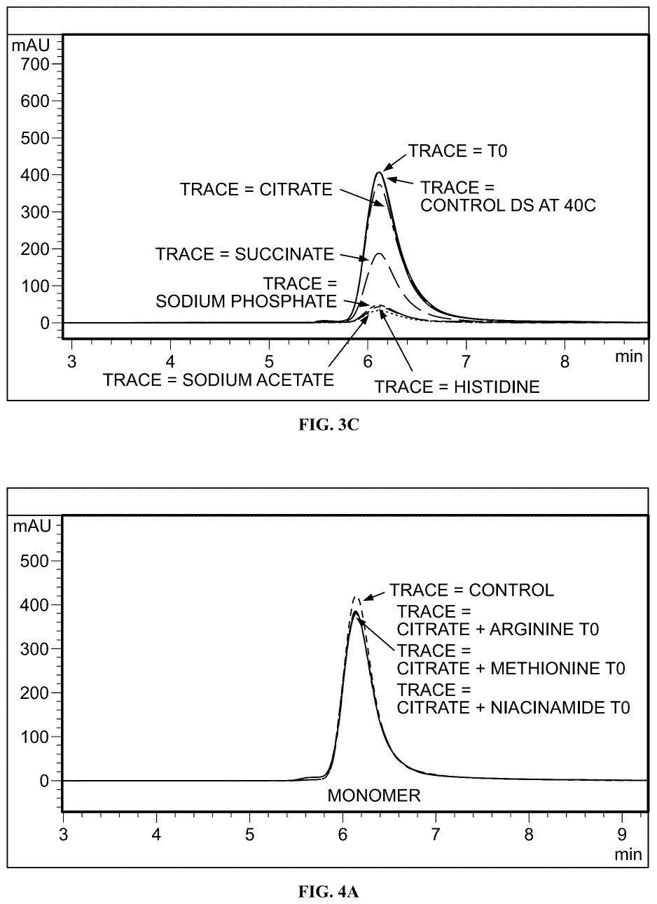

As illustrated in FIGS. 3A-3C the buffers effect on IL-12 fusion protein stability was assessed. Different buffers were tested to compare citrate, histidine, sodium phosphate, sodium acetate, and succinate buffers effects on formulation stability. As illustrated in FIG. 3A, at TO, citrate buffer yielded the best stability represented in the highest monomer recovery compared to the different buffers tested.

As illustrated in FIG. 3B two-week stability data for samples at 5° C. confirmed citrate's critical effect on stability compared to the other tested buffers.

As illustrated in FIG. 3C two-week stability data for samples at 40° C. (accelerated conditions) confirmed citrate's critical effect on stability compared to the other tested buffers.

TABLE 5
Buffers Effect on IL-12 fusion protein Stability at T0

| Buffer | % Recovery of from Control |
|---|---|
| Citrate | 96 |
| Histidine | 13 |
| Sodium Phosphate | 14 |
| Sodium Acetate | 12 |
| Succinate | 50 |

TABLE 6
Buffers Effect on IL-12 fusion protein Stability 2 Weeks at 5° C.

| | % Recovery from | % Recovery |
|---|---|---|
| Buffers | Control | from T0 |
| Control DS | NA | 99 |
| Citrate | 93 | 96 |
| Histidine | 11 | 86 |
| Sodium Phosphate | 12 | 84 |
| Sodium Acetate | 9 | 76 |
| Succinate | 46 | 91 |

TABLE 7
Buffers Effect on IL-12 fusion protein Stability 2 Weeks at 40° C.

| | % Recovery from | % Recovery |
|---|---|---|
| Buffers | Control | from T0 |
| Control DS | NA | 99 |
| Citrate | 95 | 99 |
| Histidine | 10 | 76 |
| Sodium Phosphate | 13 | 88 |
| Sodium Acetate | 8 | 71 |
| Succinate | 47 | 93 |

Summary: Based on the data for the different buffers, only citrate played a critical role in the stability of IL-12 fusion protein formulation (FIGS. 3A-3C). Percent recoveries of citrate containing formulation compared to the drug substance control remained over 90% for TO as well as for the 2-week stabilities at 5° C. and 40° C. compared to percent recoveries lower than 50% for the remaining buffers tested (Tables 5, 6 and 7). Citrate buffer has been established to be critical in maintaining the IL-12 fusion protein formulation's pH, integrity, activity, and stability at refrigerated and accelerated conditions.

### Example 4

### Effect of Citrate+Stabilizers on Formulation Stability

For the next set of experiments, different stabilizers were tested in the presence of the citrate buffer background to determine the optimal stabilizer to be combined with the citrate buffer. The different stabilizers tested were arginine, methionine, and niacinamide.

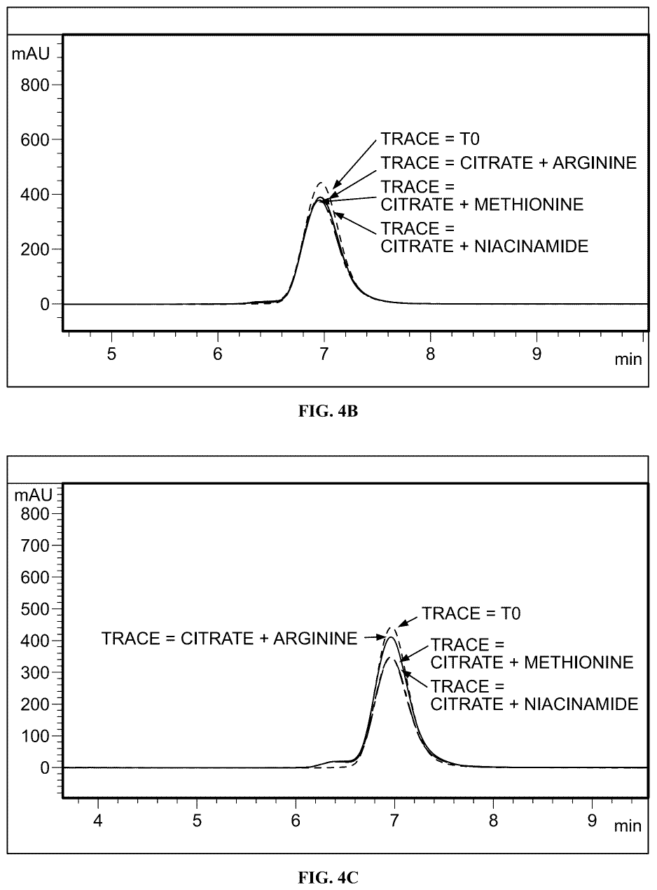

As illustrated in FIGS. 4A-4C, stabilizers effect on IL-12 fusion protein stability was assessed. Effects of arginine, methionine, or niacinamide on formulation stability were monitored based on monomer percent recovery. As illustrated in FIG. 4A, at TO, the stabilizers tested yielded comparable percent recoveries compared to the drug substance control.

As illustrated in FIG. 4B, two-week stability data for samples incubated at 5° C. yielded comparable stability results for all three stabilizers

As illustrated in FIG. 4C, when incubated the samples for two weeks under accelerated conditions of 40° C., arginine outperformed methionine and niacinamide with the highest monomer percent recovery relative to the drug substance control.

TABLE 8
Stabilizers Effect on IL-12 fusion protein Stability at T0

| Buffers | % Recovery from Control |
|---|---|
| Citrate + | 93 |
| Arginine | |
| Citrate + | 92 |
| Methionine | |
| Citrate + | 93 |
| Niacinamide | |

TABLE 9
Stabilizers Effect on IL-12 fusion protein Stability at 2 Weeks

| | % Recovery from | % Recovery from |
|---|---|---|
| Buffers | Control at 5° C. | Control at 40° C. |
| Citrate + | 92 | 99 |
| Arginine | | |
| Citrate + | 89 | 84 |
| Methionine | | |
| Citrate + | 91 | 84 |
| Niacinamide | | |

Summary: At T0, all stabilizers had the same effect on the IL-12 fusion protein monomer stability (FIG. 4A and Table 8). However, based on the accelerated conditions of 40° C. at 2 weeks (Table 9), arginine outperformed methionine and niacinamide in providing higher IL-12 fusion protein stability as evident in the percent recovery of the monomer for these samples. The percentage recovery for arginine was 99% compared to 84% for each of methionine and niacinamide. It has been established that in the presence of citrate buffer, arginine is critical for IL-12 fusion protein formulation stability at accelerated conditions, and therefore also critical it the long-term stability of the drug product.

### Example 5

### Effect of Citrate+Sugars on Formulation Stability

For the following set of experiments, different sugars were tested to confirm the optimal sugar for the IL-12 fusion protein formulation stability. The sugars tested were sucrose, lactose, and mannitol.

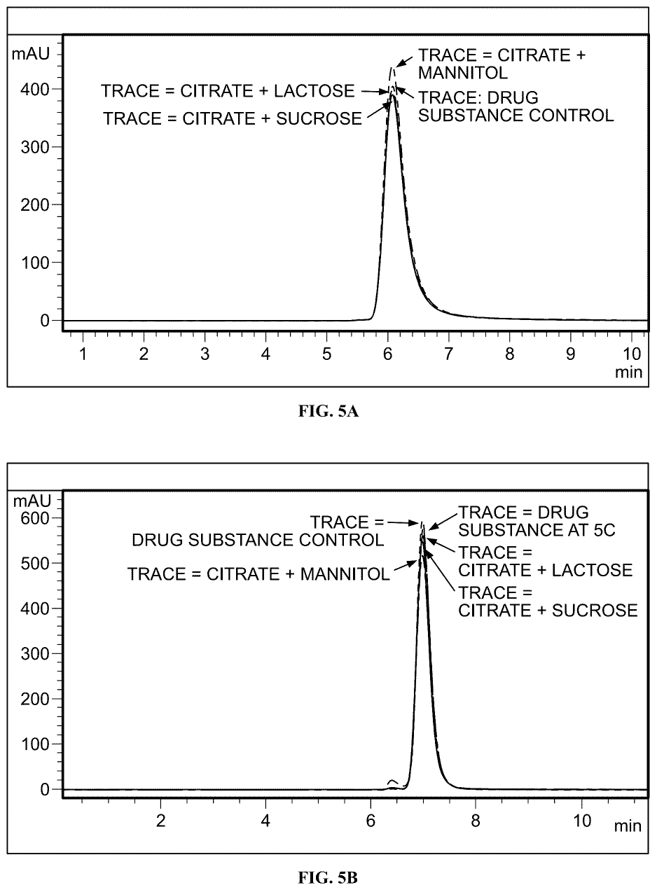

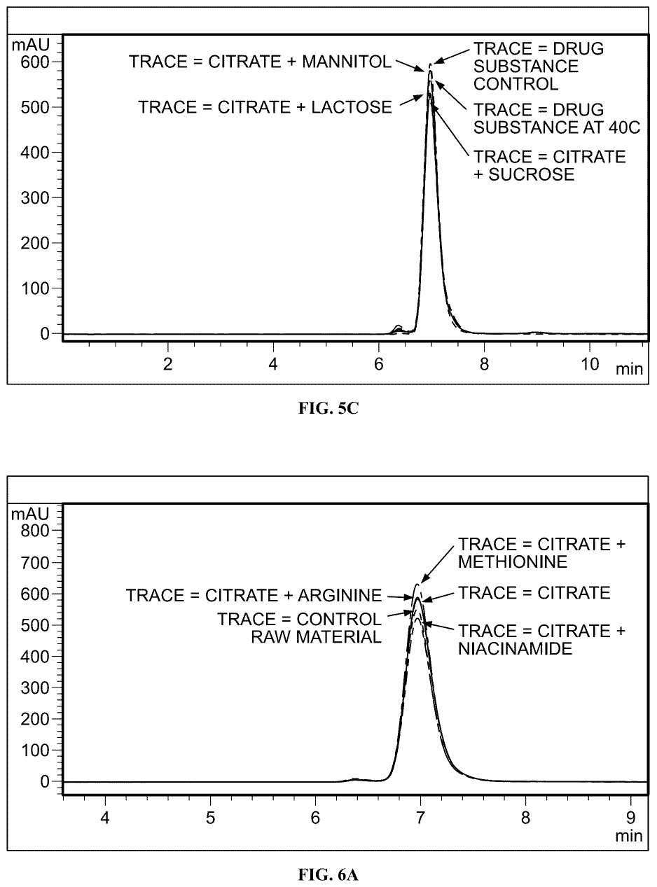

As illustrated in FIGS. 5A-5C, sugars effect on IL-12 fusion protein stability were assessed. Different sugars were tested comparing sucrose, lactose, and mannitol effects on formulation stability. As illustrated in FIG. 5A, at TO, the sugars tested yielded comparable percent recoveries compared to the drug substance (approx. 100%).

As illustrated in FIG. 5B, two-week stability data for samples at 5° C. yielded comparable stability results for all three sugars tested.

As illustrated in FIG. 5C, two-week stability data for samples at 40° C. yielded comparable stability results for all three sugars tested.

TABLE 10
Sugars Effect on IL-12 fusion protein Stability at T0

| Buffers | % Recovery from Control |
|---|---|
| Citrate + | 97 |
| Sucrose | |
| Citrate + | 100 |
| Lactose | |
| Citrate + | 111 |
| Mannitol | |

TABLE 11
Sugars Effect on IL-12 fusion protein Stability at 2 Weeks

| | % Recovery from | % Recovery from |
|---|---|---|
| Buffers | Control at 5° C. | Control at 40° C. |
| Citrate + | 93 | 93 |
| Sucrose | | |
| Citrate + | 94 | 95 |
| Lactose | | |
| Citrate + | 104 | 103 |
| Mannitol | | |

Summary: Based on the collective data for the citrate and sugar buffers, a clear advantage of one sugar was inconclusive (FIGS. 5A-5C and Tables 10 and 11). The comparable effects of the sugars could have resulted from using the drug substance that already contained surfactant in the formulation (PS20) which could have contributed to the overall stability of the tested samples shielding the effect of the different sugars tested in the formulation.

### Example 6

### SE-HPLC Analysis—Using Raw Material (RM)

The raw material (RM) of the IL-12 fusion protein was used for this part of the study. The RM of the unformulated IL-12 fusion protein was obtained post the cHA (ceramic Hydroxyapatite) chromatography. In this step of the manufacturing process, the primary goal of the purification is to remove protein aggregates as well as residual protein A ligand, host cell proteins (HCP), and DNA removal. The RM is at 2.6 mg/mL of IL-12 fusion protein and is in 5 mM sodium phosphate, 20 mM MES (2-(N-morpholino) ethanesulfonic acid), 1.4 M sodium chloride at pH 6.0. The RM is in a buffering system that can be exchanged entirely during dialysis especially due to the absence of the surfactant (PS20) making it an ideal starting material for the dialysis exchange to determine the optimal formulation excipients. For each analysis the following controls were included in addition to the different treatments: drug substance (DS) control and raw material (RM) control. The same buffers used for the DS studies were compared in the citrate buffer background in addition to the surfactant containing buffers. In addition to the DS and RM controls, citrate buffer controls were also included for reference to assess the additive effect on formulation stability.

### Example 7

### Effect of Citrate+Stabilizers on Formulation Stability

Stabilizers tested were arginine, methionine, and niacinamide. Previously with the DS studies, it was observed that arginine yielded optimal results when compared to the drug substance control outperforming methionine and niacinamide providing more stability as quantitated in the monomer percent recovery. In this set of studies, the same stabilizers are tested, however, in the presence of unformulated raw material to further confirm the results observed with the DS stability. The samples were incubated and monitored for 2 and 6 weeks at 5° C. or 40° C.

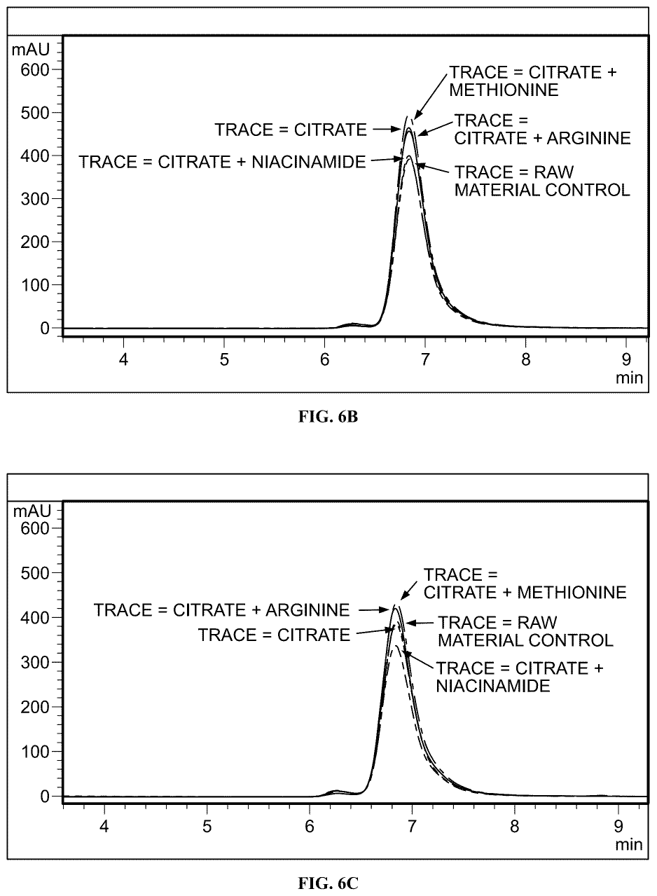

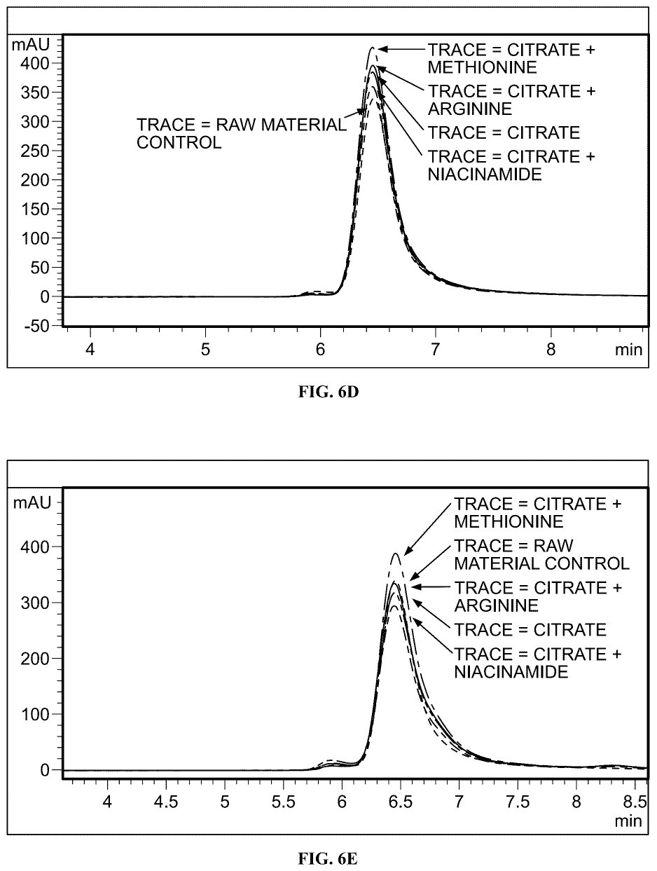

As illustrated in FIGS. 6A-6E, stabilizers effect on IL-12 fusion protein stability were assessed. Using raw material, arginine, methionine, and niacinamide effects on formulation stability were compared. As illustrated in FIG. 6A, at TO, arginine and methionine provided comparable percent recoveries of the monomer compared to the controls. Niacinamide yielded the least stability effect as evident in the lower monomer recovery compared to the citrate control.

TABLE 12
Stabilizers Effect on IL-12 fusion protein
Stability using Raw Material at T0

| | % Recovery | % Recovery | % Recovery |
|---|---|---|---|
| Buffers | from DS Ctrl | from RM Ctrl | from Citrate |
| Citrate | 97 | 106 | NA |
| Citrate + Arginine | 98 | 107 | 101 |
| Citrate + Methionine | 105 | 115 | 108 |
| Citrate + Niacinamide | 87 | 95 | 89 |

As illustrated in FIG. 6B, when incubated at 5° C. for two weeks, arginine and methionine continue to provide comparable results where niacinamide yielded the lowest IL-12 fusion protein monomer percent recovery compared to the citrate control.

As illustrated in FIG. 6C, when incubated at 40° C. for two weeks, arginine and methionine continue to provide comparable results where niacinamide yielded the lowest IL-12 fusion protein monomer percent recovery compared to the citrate control.

As illustrated in FIG. 6D, when incubated at 5° C. for six weeks, arginine and methionine continue to provide comparable results where niacinamide yielded the lowest IL-12 fusion protein monomer percent recovery compared to the citrate control.

As illustrated in FIG. 6E, when incubated at 40° C. for six weeks, arginine and methionine continued to provide comparable results where niacinamide yielded the lowest IL-12 fusion protein monomer percent recovery compared to the citrate control.

TABLE 13
Stabilizers Effect on IL-12 fusion protein Stability
using Raw Material 2 Weeks at 5° C.

| | % Recovery | % Recovery | % Recovery | % Recovery from |
|---|---|---|---|---|
| Buffers | from DS Ctrl | from RM Ctrl | from T0 | Citrate Ctrl |
| RM at 5° C. | 90 | 105 | 93 | 90 |
| Citrate | 100 | 117 | 97 | NA |
| Citrate + Arginine | 98 | 114 | 95 | 98 |
| Citrate + Methionine | 106 | 124 | 96 | 106 |
| Citrate + Niacinamide | 85 | 100 | 93 | 86 |

TABLE 14
Stabilizers Effect on IL-12 fusion protein Stability
using Raw Material 2 Weeks at 40° C.

| | % Recovery | % Recovery | % Recovery | % Recovery from |
|---|---|---|---|---|
| Buffers | from DS Ctrl | from RM Ctrl | from T0 | Citrate Ctrl |
| RM at 40° C. | 82 | 95 | 84 | 95 |
| Citrate | 86 | 100 | 84 | NA |
| Citrate + Arginine | 92 | 107 | 89 | 107 |
| Citrate + Methionine | 96 | 112 | 87 | 112 |
| Citrate + Niacinamide | 75 | 88 | 82 | 88 |

TABLE 15
Stabilizers Effect on IL-12 fusion protein Stability
using Raw Material 6 Weeks at 5° C.

| | | | | % Recovery |
|---|---|---|---|---|
| | % Recovery | % Recovery | | from |
| | from DS | from RM | % Recovery | Citrate |
| Buffers | Ctrl | Ctrl | from T0 | Ctrl |
| RM at 5° C. | 84 | 105 | 84 | 95 |
| Citrate | 88 | 110 | 84 | NA |
| Citrate + | 91 | 114 | 86 | 103 |
| Arginine | | | | |
| Citrate + | 98 | 123 | 86 | 111 |
| Methionine | | | | |
| Citrate + | 83 | 103 | 88 | 93 |
| Niacinamide | | | | |

TABLE 16
Stabilizers Effect on IL-12 fusion protein Stability
using Raw Material 6 Weeks at 40° C.

| | | | | % Recovery |
|---|---|---|---|---|
| | % Recovery | % Recovery | | from |
| | from DS | from RM | % Recovery | Citrate |
| Buffers | Ctrl | Ctrl | from T0 | Ctrl |
| RM at 40° C. | 78 | 97 | 78 | 94 |
| Citrate | 83 | 104 | 79 | NA |
| Citrate + | 86 | 108 | 81 | 104 |
| Arginine | | | | |
| Citrate + | 98 | 122 | 86 | 118 |
| Methionine | | | | |
| Citrate + | 75 | 94 | 80 | 91 |
| Niacinamide | | | | |

TABLE 17
High Molecular Weight Species (HMW) Fold Increase
Compared to T0 for Samples incubated at 40° C.)

| Buffers | 2 Weeks | 6 Weeks |
|---|---|---|
| RM at 40° C. | 1 | 2 |
| Citrate | 2 | 3 |
| Citrate + Arginine | 1 | 1 |
| Citrate + Methionine | 2 | 2 |
| Citrate + Niacinamide | 3 | 2 |

Summary: Different stabilizers were tested using the IL-12 fusion protein raw material to test the optimal stabilizer for the formulation. At TO, arginine and methionine yielded the highest percentage recoveries compared to the raw material control (FIG. 6A and Table 12). Both stabilizers, arginine and methionine, also had the highest percent recoveries when compared to the DS formulation of IL-12 fusion protein at 98% and 105%, respectively. Moreover, when comparing the two to the citrate control, arginine and methionine performed best in terms of monomer percent recoveries, 101% and 108%, while niacinamide yielded the lowest percentage recovery, 89% (Table 12). The same trend was observed throughout the study for the different timepoints at 2 weeks and 6 weeks when samples were incubated at 5° C. or 40° C. Although, methionine's monomer percentage recoveries were higher than arginine's (FIGS. 6D-6E), they both yielded percentages over 100% relative to the RM control and the citrate control after 6 weeks incubation period (Tables 15 and 16). Because arginine's and methionine's effects on stability were comparable, a closer look into their effect on the high molecular weight (HMW) species stability of the formulation was investigated for the samples at accelerated conditions (40° C.). It was observed that at 2 and 6 weeks, buffer containing methionine led to a two-fold increase in HMW species compared to the formulation containing arginine (Table 17). Based on these observations, it was concluded that arginine is superior in maintaining IL-12 fusion protein stability in terms of monomer percent recovery as well as limiting HMW species induction. In the presence of citrate buffer, arginine has been demonstrated to be critical for IL-12 fusion protein stability leading to decreased aggregations and therefore higher formulation uniformity and stability.

### Example 8

### Effect of Citrate+Sugars on Formulation Stability

The same sugars tested with the DS were tested again side by side using the raw material to confirm the optimal sugar for the IL-12 fusion protein formulation stability. The sugars tested were sucrose, lactose, and mannitol. Previously with the DS study, all sugars were comparable. For this set of experiments, utilizing the raw material, new observations were made. First, upon removing the lactose containing sample from the dialysis bag and transferring to a glass vial, it was noted that the formulation turned milky/cloudy when mixed whereas with sucrose, mannitol, or citrate alone, the formulations remained clear. Based on this observation, the lactose-containing buffer was excluded from analysis due to the milky and cloudy appearance. Hence, for the next set of experiments only sucrose and mannitol were investigated.

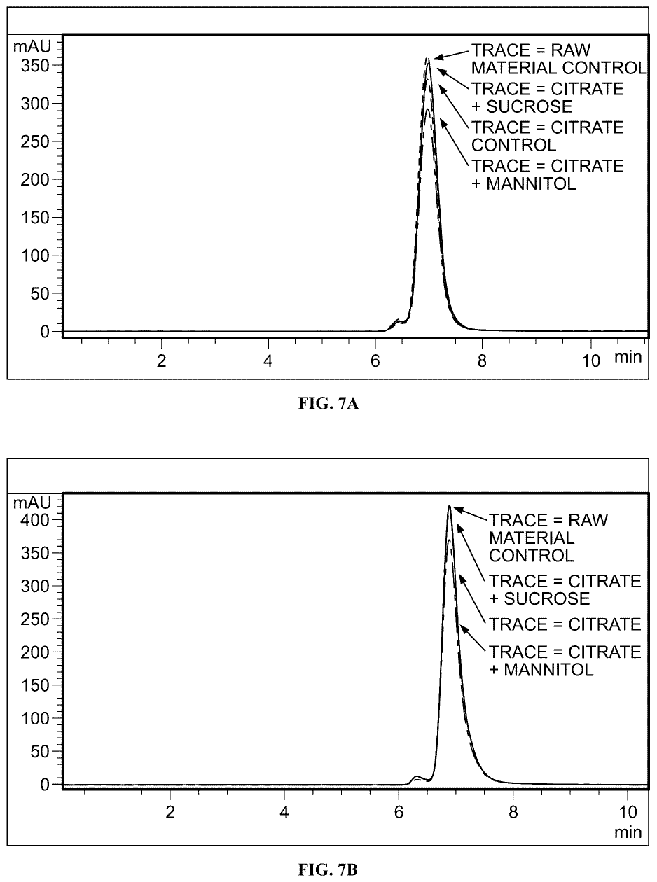

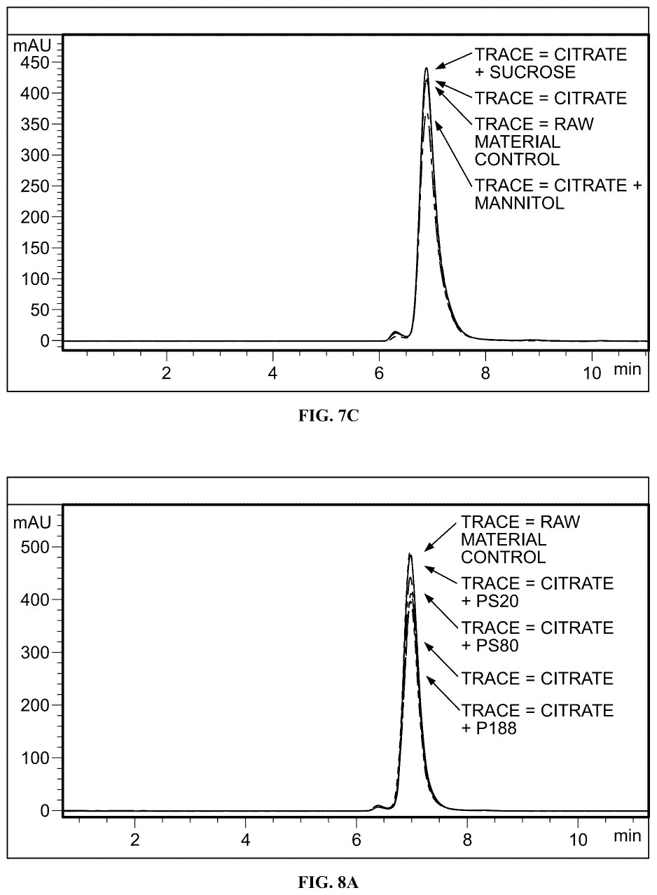

FIGS. 7A-7C illustrate sugars effect on IL-12 fusion protein stability. Sucrose and mannitol effects on formulation stability were compared. As illustrated in FIG. 7A, at TO, it was evident that sucrose contributed to the formulation stability compared to mannitol.

As illustrated in FIG. 7B, after two weeks incubation at 5° C., sucrose continued to provide higher stability for IL-12 fusion protein compared to mannitol.

As illustrated in FIG. 7C, after two weeks incubation at 40° C., sucrose continued to provide higher stability for IL-12 fusion protein compared to mannitol.

TABLE 18
Sugars Effect on IL-12 fusion protein
Stability using Raw Material at T0

| | % Recovery | % Recovery |
|---|---|---|
| Buffers | from DS Ctrl | from RM Ctrl |
| RM | 84 | NA |
| material | | |
| T0 | | |
| Citrate | 76 | 90 |
| Citrate + | 81 | 96 |
| Sucrose | | |
| Citrate + | 67 | 80 |
| Mannitol | | |

TABLE 19
Sugars Effect on IL-12 fusion protein Stability
using Raw Material 2 Weeks at 5° C.

| Citrate + | % Recovery | % Recovery | % Recovery | % Recovery of |
|---|---|---|---|---|
| Sugars | of DS Ctrl | of RM Ctrl | of T0 | Citrate Ctrl |
| RM at 5° C. | 92 | 106 | 110 | 109 |
| Citrate | 84 | 97 | 111 | NA |
| Citrate + | 85 | 99 | 107 | 102 |
| Sucrose | | | | |
| Citrate + | 75 | 87 | 114 | 90 |
| Mannitol | | | | |

TABLE 20
Sugars Effect on IL-12 fusion protein Stability
using Raw Material 2 Weeks at 40° C.

| | % Recovery | % Recovery | | % Recovery |
|---|---|---|---|---|
| Citrate + | of DS | of RM | % Recovery | of Citrate |
| Sugars | Ctrl | Ctrl | of T0 | Ctrl |
| RM at 40° C. | 91 | 105 | 109 | 101 |
| Citrate | 90 | 104 | 119 | NA |
| Citrate + | 93 | 108 | 116 | 104 |
| Sucrose | | | | |
| Citrate + | 78 | 91 | 118 | 87 |
| Mannitol | | | | |

Summary: Based on the data collected for sucrose and mannitol containing buffers, it was observed that sucrose provided more stability and clearly contributed positively to formulation stability (FIGS. 7A-7C). Sucrose led to the highest monomer percent recoveries when compared to the DS or RM controls as well as the citrate control. These observations were consistent for sucrose across the different timepoints and at different incubation temperatures (Tables 18, 19 and 20). Mannitol is therefore excluded due to its low monomer percent recoveries compared to sucrose. Moreover, the lactose-containing buffer led to precipitation as was evident from the observed cloudy appearance leading to its exclusion from the study. Therefore, sucrose was confirmed as the optimal sugar for use in the IL-12 fusion protein formulation. In the presence of citrate buffer, sucrose has been established as critical for maintaining IL-12 fusion protein stability at refrigerated and accelerated conditions in addition to maintaining the formulation's isotonicity.

### Example 9

### Effect of Citrate+Surfactants on Formulation Stability

Surfactants effects on the IL-12 fusion protein formulation were investigated by testing different commonly used surfactants; PS20, PS80, and P188 in presence of the citrate buffer background. The RM was used for this set of experiments, whereas DS was not used previously because it had already contained the PS20. The samples were incubated for up to 5 weeks at either 5° C. or 40° C. and stability was assessed for the samples in terms of the monomer percent recoveries.

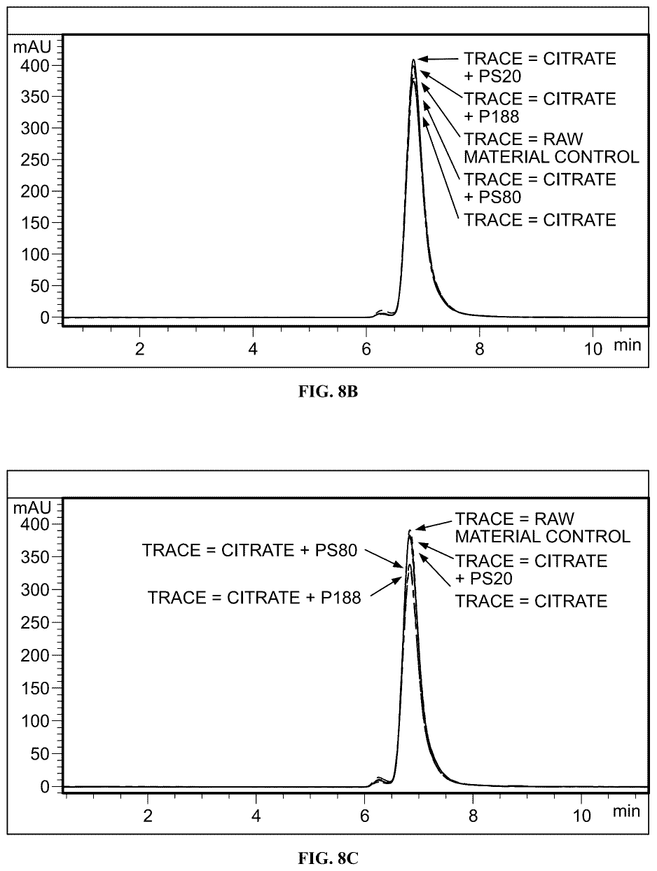

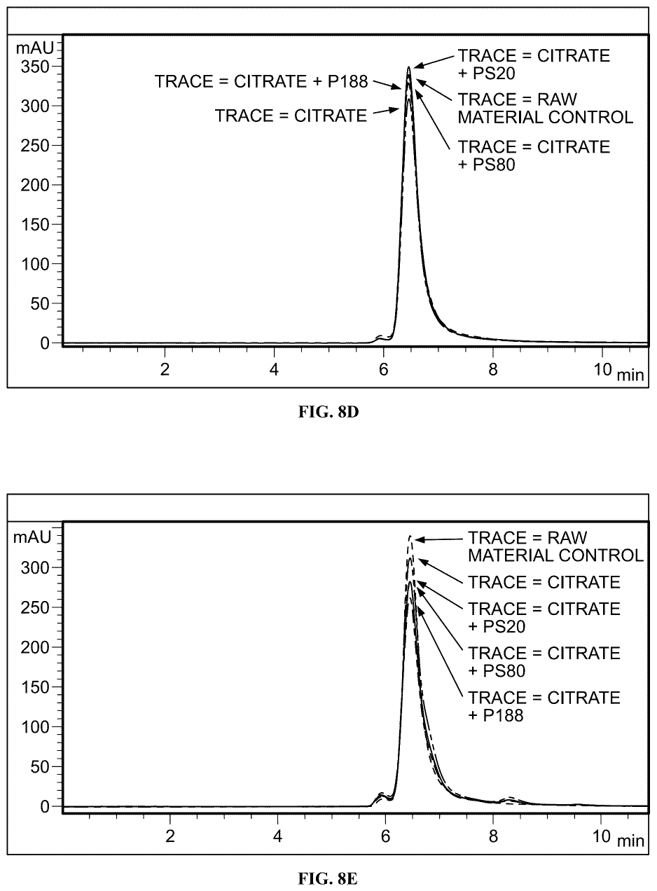

As illustrated in FIGS. 8A-8E, surfactants effect on IL-12 fusion protein stability were assessed. Using the raw material, PS20, PS80, and P188 effects on formulation stability were compared. As illustrated in FIG. 8A, at TO, PS20 provided higher stability for the formulation compared to PS80 or P188 relative to the raw material control; 97% monomer recovery compared to 88% and 79%, respectively.

As illustrated in FIG. 8B, after a week incubation at 5° C., the three surfactants' effects on stability were comparable; ≥95% monomer recoveries compared to the raw material control.

As illustrated in FIG. 8C, after a week of incubation at 40° C., PS20 provided superior stability effects for IL-12 fusion protein under accelerated conditions compared to PS80 and P188. PS20 yielded monomer percent recovery of 98% relative to the raw material control compared to 87% and 85% for PS80 and P188, respectively.

As illustrated in FIG. 8D, after five weeks incubation at 5° C., the three surfactants' effects on stability remained comparable; ≥95% compared to the raw material control.

As illustrated in FIG. 8E, after five weeks incubation at 40° C., both PS20 and PS80 yielded equal monomer percent recovery of 92% relative to the raw material control compared to 83% for P188.

TABLE 21
Surfactants Effect on IL-12 fusion protein
Stability using Raw Material at T0

| | % Recovery | % Recovery | % Recovery from |
|---|---|---|---|
| Buffers | from DS Ctrl | from RM Ctrl | Citrate Ctrl |
| RM material T0 | 90 | NA | 122 |
| Citrate | 74 | 82 | NA |
| Citrate + PS20 | 87 | 97 | 118 |
| Citrate + PS80 | 80 | 88 | 107 |
| Citrate + P188 | 71 | 79 | 96 |

TABLE 22
Surfactants Effect on IL-12 fusion protein
Stability using Raw Material 1 Week at 5° C.

| | | | | % Recovery |
|---|---|---|---|---|
| | % Recovery | % Recovery | | from |
| | from DS | from RM | % Recovery | Citrate |
| Buffers | Ctrl | Ctrl | from T0 | Ctrl |
| RM at 5° C. | 93 | 109 | 101 | 116 |
| Citrate | 80 | 94 | 105 | NA |
| Citrate + PS20 | 87 | 102 | 98 | 109 |
| Citrate + PS80 | 81 | 95 | 99 | 101 |
| Citrate + P188 | 85 | 100 | 116 | 106 |

TABLE 23
Surfactants Effect on IL-12 fusion protein Stability
using Raw Material 1 Week at 40° C.

| | | | | % Recovery |
|---|---|---|---|---|
| | % Recovery | % Recovery | | from |
| | from DS | from RM | % Recovery | Citrate |
| Buffers | Ctrl | Ctrl | from T0 | Ctrl |
| RM at 40° C. | 84 | 98 | 91 | 115 |
| Citrate | 73 | 85 | 96 | NA |
| Citrate + PS20 | 84 | 98 | 94 | 115 |
| Citrate + PS80 | 74 | 87 | 91 | 102 |
| Citrate + P188 | 72 | 85 | 99 | 99 |

TABLE 24
Surfactants Effect on IL-12 fusion protein Stability
using Raw Material 5 Weeks at 5° C.

| | | | | % Recovery |
|---|---|---|---|---|
| | % Recovery | % Recovery | | from |
| | from DS | from RM | % Recovery | Citrate |
| Buffers | Ctrl | Ctrl | from T0 | Ctrl |
| RM at 5° C. | 87 | 109 | 91 | 122 |
| Citrate | 71 | 89 | 91 | NA |
| Citrate + PS20 | 80 | 100 | 87 | 113 |
| Citrate + PS80 | 78 | 97 | 93 | 109 |
| Citrate + P188 | 76 | 95 | 101 | 107 |

TABLE 25
Surfactants Effect on IL-12 fusion protein Stability
using Raw Material 5 Weeks at 40° C.

| | | | | % Recovery |
|---|---|---|---|---|
| | % Recovery | % Recovery | | from |
| | from DS | from RM | % Recovery | Citrate |
| Buffers | Ctrl | Ctrl | from T0 | Ctrl |
| RM at 40° C. | 81 | 102 | 86 | 97 |
| Citrate | 84 | 105 | 107 | NA |
| Citrate + PS20 | 74 | 92 | 80 | 88 |
| Citrate + PS80 | 74 | 92 | 88 | 87 |
| Citrate + P188 | 67 | 83 | 88 | 79 |

Summary: Based on the data, it was observed that PS20 provided the highest stability to the formulation compared to PS80 or P188 at TO (FIG. 8A). The monomer percent recovery was 97% for PS20 compared to 88% and 79% for PS80 and P188, respectively relative to the RM control (Table 21). Overall incubation at 5° C. for 1 or 5 weeks showed comparable stability results for all three surfactants tested (Tables 22 and 24). However, when investigating the surfactant's effects at accelerated conditions, it was observed that after 1 week incubation at 40° C. PS20 provided more monomer stability at 98% relative to RM compared to the other two contenders at 87% and 85% (Table 23). At 5 weeks incubation at 40° C., PS20 and PS80 showed comparable percent recovery results while P188 remained low and provided the least formulation stability (Table 25). Based on these findings and especially based on the T0 and week 1 results and the inconsistencies of PS80, PS20 is considered the most optimal surfactant for the IL-12 fusion protein formulation stability. In the presence of citrate buffer, PS20 has been shown to provide maximal physical and chemical stability at accelerated conditions for IL-12 fusion protein leading to a stable and uniform formulation. This formulation additive has therefore been established as essential to long term drug stability at refrigerated temperatures.

### Example 10

### Protein Concentration Analysis

### Protein Concentration Analysis of Citrate+Stabilizers

To further study the formulation of IL-12 fusion protein, the raw material was exchanged in citrate buffer in the presence of various stabilizers. Volume after dialysis of raw material in citrate buffer control with or without stabilizers was in the range of 1.15-1.3 mL (Table 26). The ratio of OD₂₆₀ and OD₂₈₀ was recorded during the protein concentration analysis. The OD₂₆₀/OD₂₈₀ value represents the purity of protein according to Spectrophotometer DeNovix's instruction and the pure protein samples should have a ratio below 0.57. The OD₂₆₀/OD₂₈₀ of samples obtained from citrate buffer control, citrate/arginine and citrate/methionine were 0.55-0.56, whereas that of sample obtained from citrate/niacinamide was −0.19 (Table 27). The % recovery of samples obtained from citrate buffer control and citrate/arginine buffer was about 92-111% at TO and remained stable when held at 5° C. and 40° C. for 2 weeks. The % recovery of samples obtained from citrate/niacinamide buffer was the lowest at TO and did not change when held at 5° C. and 40° C. for 2 weeks. Interestingly, the % recovery of sample obtained from citrate/methionine buffer increased by almost 20% after dialysis at TO and when held at 5° C. and 40° C. (Table 28).

TABLE 26
Volume and Protein Concentration of Samples Obtained
from Various Stabilizer Buffers

| | Volume post | Protein Concentration (mg/mL) | | | | |
|---|---|---|---|---|---|---|
| | Dialysis | | Week 1 | | Week 2 | |
| Buffer | (mL) | T0 | 5° C. | 40° C. | 5° C. | 40° C. |
| RM Ctrl | NA | NA | 2.943 | | 1.903 | |
| RM | NA | 2.918 | 2.997 | 2.989 | 2.135 | 2.109 |
| Citrate | 1.3 | 3.229 | 3.154 | 3.214 | 2.183 | 2.176 |
| Citrate + | 1.25 | 3.176 | 3.123 | 3.213 | 2.164 | 2.203 |
| Arginine | | | | | | |
| Citrate + | 1.3 | 3.423 | 3.478 | 3.479 | 2.098 | 2.269 |
| Methionine | | | | | | |
| Citrate + | 1.15 | 2.686 | 2.720 | 2.722 | 1.993 | 2.121 |
| Niacinamide | | | | | | |

TABLE 27
The OD₂₆₀/OD₂₈₀ of Samples after
Stabilizer Buffer Exchange at T0

| Buffer | OD₂₆₀/OD₂₈₀ |
|---|---|
| RM | 0.55 |
| Citrate | 0.55 |
| Citrate + Arginine | 0.55 |
| Citrate + Methionine | 0.56 |
| Citrate + Niacinamide | −0.19 |

TABLE 28
The % Recovery of Samples Obtained from Various Stabilizer
Buffers at 5° C. and 40° C.

% Recovery from RM Ctrl

| | |
|---|---|
| Week 1 | Week 2 |

| Buffer | T0 | 5° C. | 40° C. | 5° C. | 40° C. |
|---|---|---|---|---|---|
| RM | 100.0 | 101.8 | 101.6 | 112.2 | 110.8 |
| Citrate | 110.7 | 107.2 | 109.2 | 114.7 | 114.4 |
| Citrate + | 108.9 | 106.1 | 109.2 | 113.7 | 115.7 |
| Arginine | | | | | |
| Citrate + | 117.3 | 118.2 | 118.2 | 110.3 | 119.2 |
| Methionine | | | | | |
| Citrate + | 92.1 | 92.4 | 92.5 | 104.7 | 111.4 |
| Niacinamide | | | | | |

### Protein Concentration Analysis of Citrate+Sugars

Dialyzed samples obtained from various sugar exchange buffers were collected and recorded. The samples from citrate buffer control and citrate/mannitol buffer had a volume of 1.2 mL, whereas citrate/sucrose sample had a volume of 1 mL. Next, protein concentration was analyzed and standardized based on recorded volume (Table 29). At T0, the % recoveries of all samples obtained from citrate buffer control and citrate/sucrose were within the range of 96-109%. However, the % recovery of sample obtained from citrate/mannitol buffer was only 87% at T0 and decreased to 79% in week 1 and 22% in week 2 when held at 5° C. and 40° C., respectively. (Table 30).

TABLE 29
Volume and Protein Concentration of Samples Obtained
from Various Sugar Buffers

| | Volume | | | | | |
|---|---|---|---|---|---|---|
| | post | Protein Concentration (mg/mL) | | | | |
| | Dialysis | | Week 1 | | Week 2 | |
| Buffer | (mL) | T0 | 5° C. | 40° C. | 5° C. | 40° C. |
| RM Ctrl | NA | NA | 3.295 | | 2.031 | |
| RM | NA | 3.123 | 2.816 | 2.839 | 2.055 | 2.057 |
| Citrate | 1.2 | 2.990 | 2.892 | 3.163 | 2.018 | 2.173 |
| Citrate + | 1.0 | 3.413 | 3.330 | 3.462 | 2.267 | 2.177 |
| Sucrose | | | | | | |
| Citate + | 1.2 | 3.090 | 3.008 | 3.059 | 2.078 | 2.134 |
| Mannitol | | | | | | |

TABLE 30
% Recovery of Samples Obtained from Various Sugar Buffers
when Held at 5° C. and 40° C.

% Recovery from RM Ctrl

| | |
|---|---|
| Week 1 | Week 2 |

| Buffer | T0 | 5° C. | 40° C. | 5° C. | 40° C. |
|---|---|---|---|---|---|
| RM | NA | 85.5 | 86.2 | 101.2 | 101.3 |
| Citrate | 95.7 | 87.8 | 96.0 | 99.4 | 107.0 |
| Citrate + | 109.3 | 101.1 | 105.1 | 111.6 | 107.2 |
| Sucrose | | | | | |
| Citate + | 86.5 | 79.4 | 79.8 | 22.4 | 22.3 |
| Mannitol | | | | | |

### Protein Concentration Analysis of Citrate+Surfactants

Next, the raw material was exchanged in citrate buffer in the presence of various surfactants. Volume after dialysis of raw material in citrate buffer control with or without surfactants was in the range of 1.10-1.15 mL (Table 31). Dialyzed sample obtained from citrate/P188 buffer had % recovery the lowest when held at 5° C. and 40° C. The % recovery of sample obtained from citrate buffer control, citrate/PS20 and citrate/PS80 was about 86-93% at T0 and about 93-104% and 96-101% when held at 5° C. and 40° C. for 1 week, respectively (Table 32).

TABLE 31
Volume and Protein Concentration of Samples Obtained
from Various Surfactant Buffers

| | Volume | | | | | |
|---|---|---|---|---|---|---|
| | after | Protein Concentration (mg/mL) | | | | |
| | Dialysis | | Week 1 | | Week 2 | |
| Buffer | (mL) | T0 | 5° C. | 40° C. | 5° C. | 40° C. |
| RM Ctrl | NA | NA | 1.903 | | 2.044 | |
| RM | NA | 2.962 | 2.026 | | 2.038 | |
| Citrate | 1.10 | 2.539 | 1.760 | 1.824 | 2.000 | 2.077 |
| Citrate + | 1.15 | 2.703 | 1.973 | 1.930 | 1.800 | 1.795 |
| PS20 | | | | | | |
| Citrate + | 1.15 | 2.751 | 1.879 | 1.909 | 1.902 | 2.000 |
| PS80 | | | | | | |
| Citrate + | 1.10 | 2.665 | 1.582 | 1.553 | 1.880 | 1.892 |
| P188 | | | | | | |

TABLE 32
The % Recovery of Samples Obtained from Various Surfactant
Buffers when Held at 5° C. and 40° C.

% Recovery from RM Ctrl

| | |
|---|---|
| Week 1 | Week 2 |

| Buffer | T0 | 5° C. | 40° C. | 5° C. | 40° C. |
|---|---|---|---|---|---|
| RM | NA | 106.5 | 107.1 | 97.8 | 101.6 |
| Citrate | 85.7 | 92.5 | 95.8 | 88.0 | 87.8 |
| Citrate + | 91.2 | 103.7 | 101.4 | 93.1 | 97.8 |
| PS20 | | | | | |
| Citrate + | 92.9 | 98.7 | 100.3 | 92.0 | 92.6 |
| PS80 | | | | | |
| Citrate + | 90.0 | 83.1 | 81.6 | 87.7 | 107.3 |
| P188 | | | | | |

### Example 11

### Discussion and Conclusions

The IL-12 fusion protein is an antibody-cytokine recombinant fusion protein that is designed to target IL-12 to areas of tumor necrosis enhancing the T cell infiltration and proliferation in the tumor microenvironment. IL-12 fusion protein is under investigation to be combined with other immunotherapies for its potent and robust anticancer benefits. Therefore, to confirm the optimal IL-12 fusion protein formulation, different studies were conducted to test different buffers to optimize the formulation and ensure that the current formulation is indeed the most stable and optimal.

Different buffers were first tested on the drug substance and raw material of IL-12 fusion protein using buffer exchange techniques where the samples were subjected to two short dialysis sessions followed by an overnight dialysis session to ensure complete buffer exchange. Samples were then collected and analyzed using SE-HPLC for samples at T0 (right after removing from dialysis) as well as at different timepoints. Protein concentration was also determined for all samples at different time points to further confirm the data. For the initial DS studies, it was observed that indeed citric acid provided the highest stability for IL-12 fusion protein at TO and across the different time points and temperatures (FIGS. 3A-3C). The IL-12 fusion protein monomer percent recoveries for the citrate buffer samples remained over 90% relative to the DS control for all timepoints compared to only 50% and lower for the second-best contender which was succinate (Tables 5, 6, and 7). Hence, the citrate buffer was selected and confirmed as the optimal buffering background for IL-12 fusion protein formulation. For the remaining studies, citrate buffer background was used, and the different stabilizers, sugars, or surfactants were tested. For the DS studies using stabilizers, initially all tested stabilizers performed similarly (FIGS. 4A and 4B), however under accelerated conditions arginine was shown to provide the highest stability (FIG. 4C and Table 9). Testing the different sugars also yielded comparable results between the different sugars where all three provided similar stability results (FIGS. 5A and 5B, Table 11).

Further studies were conducted using the RM to further confirm the data observed with the DS material as well as to test the different surfactants. Testing the same stabilizers in the citrate buffer background showed the best stability results for buffers containing arginine and methionine (FIGS. 6A-6E). While the stability results were comparable between the two and slightly higher for methionine (Tables 15 and 16), arginine provided more stability for the HMW species compared with methionine (Table 17). Higher HMW species result from protein aggregates, through weak nonspecific interactions or covalent bonds, which could significantly impact stability, safety and efficacy of therapeutic proteins. Therefore, particular care should be taken in selecting the excipients that would lead to the least HMW species occurrence. Moreover, when comparing the different sugars in the citrate buffer, lactose was excluded due to precipitation and cloudy appearance of the formulation. When sucrose and mannitol were compared, it was evident that sucrose aided in a more stable IL-12 fusion protein formulation and clearly surpassed mannitol in stability effects (FIGS. 7A-7C and Tables 18, 19 and 20). Finally, when analyzing and comparing the surfactants in the presence of the citrate buffer, PS20 had yielded higher IL-12 fusion protein stability in terms of monomer percent recoveries at TO and week 1 timepoint under accelerated conditions (FIGS. 8A and 8C and Tables 21 and 23). However, by week 5 under the accelerated conditions, it was observed that PS20 and PS80 yielded comparable stability results for IL-12 fusion protein formulation (FIG. 8E and Table 25). Either PS20 or PS80 are acceptable surfactants, however, based on the T0 and week 1 consistent results under accelerated conditions, PS20 was selected as the optimal surfactant for the IL-12 fusion protein stability in terms of consistency. In addition to the SE-HPLC data, during the surfactants-containing buffers preparation, it was observed that PS20 dissolved considerably better and faster than PS80 presenting yet another advantage to using PS20 over PS80 for formulation and manufacturing. Additionally, the pH was kept at 6.0 for all tested conditions as it is close to 1 pH unit below the pI of IL-12 fusion protein (pI 6.73), maintaining the pH as close as possible to the physiological conditions and minimizing irritation and tissue damage at the injection site as a result. Typically, where the pH of a formulation is one or more pH units above or below the pI of the protein, this helps reduce aggregation. Additionally, while neutral to mildly basic conditions favor deamidation, mildly acidic conditions markedly slow down the process, hence pH 6.0 was selected.

In addition to SE-HPLC analysis, protein concentrations were performed. The protein purity of sample obtained from citrate/niacinamide buffer was low based on the value of OD₂₆₀/OD₂₈₀. The percent recovery of sample obtained from citrate/methionine buffer increased by almost 20% after dialysis and when held at 5° C. and 40° C. Only the sample obtained from citrate/arginine buffer was stable at 5° C. and 40° C. Therefore, arginine prolongs the stability of IL-12 fusion protein compared to methionine and niacinamide. Next, the formulation with various sugars was performed. The sample obtained from citrate/sucrose buffer had a stable protein concentration when held at 5° C. and 40° C. However, the protein concentration of sample obtained from citrate/mannitol buffer decreased. Therefore, the formulation with sucrose as a tonicity agent is the best compared to mannitol. IL-12 fusion protein needs to be stabilized using stabilizer and surfactant. Surfactants are used to prevent protein adsorption and oligomerization. Citrate buffer with PS20, PS80, or P188 was studied. The sample obtained from citrate with P188 had the lowest protein concentration compared to that obtained from citrate buffer with PS20 or PS80. Therefore, PS20 or PS80 is suitable as a surfactant for IL-12 fusion protein formulation. However, based on the SE-HPLC data, PS20 was shown to provide the most consistent stability results.

Based on these results, the following excipients were found to contribute the highest stability for IL-12 fusion protein formulation in the citrate buffer background: arginine, sucrose, and polysorbate 20. The findings were confirmed by the exceptional and unexpected 5-year stability data for this formulation when stored at 2-8° C. Under long-term conditions, 60-months data are available for historical drug product (DP) batches Y17A8880, NH1D001, and NH1E001, NH1G001 and NH1H001. The comprehensive stability data highlighted in Tables 33-41 indicated IL-12 fusion protein's stability for at least 60 months (5 years) when stored at 2-8° C. Additionally per the ICH Q1E (Evaluation for Stability Data), shelf life can be extended for no more than 12 months beyond the period covered by the long-term data, therefore the IL-12 fusion protein DP is indeed stable for a period of 6 years. Moreover, the IL-12 fusion protein bulk drug substance (BDS) is stable when stored frozen at −80° C. for at least 48 months; see Tables 42 and 43).

The experimental findings, combined with the outstanding and unexpected 6-year stability data, for an antibody drug conjugate stored at 2-8° C., underscore the superior performance and stability of the IL-12 fusion protein formulation, which is IL-12 fusion protein formulated at 1.5 mg/mL in 10 mM citric acid monohydrate, 100 mM L-arginine monohydrate, 100 mM sucrose, 0.05% (w/w) polysorbate 20 at pH 6.0.

### Table 33: Long Term Storage Stability of IL-12 Fusion Protein at 5±3° C.

The testing frequencies for the long-term storage conditions at 5±3° C. are summarized in Table 33 below.

TABLE 33
Testing Frequency for IL-12 fusion protein DP Batches under Long Term
Storage Conditions (5 ± 3° C.)

| | Time (Months) | | | | | | | | | | | | |
|---|---|---|---|---|---|---|---|---|---|---|---|---|---|
| Test Parameter | 0 | 3 | 6 | 9 | 12 | 18 | 24 | 30 | 36 | 42 | 48 | 54 | 60 |
| Appearance | X | X | X | X | X | X | X | X | X | X | X | X | X |
| Degree of Coloration | X | X | X | X | X | X | X | X | X | — | X | — | X |
| Clarity and Degree of Opalescence | X | — | X | — | X | — | X | — | X | — | X | — | X |
| pH | X | — | X | — | X | — | X | — | X | X | X | — | X |
| Osmolality | X | — | X | — | X | — | X | — | X | — | X | — | X |
| Extractable Volume | X | — | X | — | X | — | X | — | X | — | X | — | X |
| Subvisible Particles | X | — | X | — | X | — | X | — | X | X | X | X | X |
| Protein Content by O.D. | X | X | X | X | X | X | X | X | X | X | X | X | X |
| Specific/Biological Activity | X | X | X | X | X | X | X | X | X | X | X | X | X |
| DNA Binding Activity | X | X | X | X | X | X | X | X | X | X | X | X | X |
| High Molecular Weight Related | X | X | X | X | X | X | X | X | X | X | X | X | X |
| Substances by SE-HPLC | | | | | | | | | | | | | |
| Low Molecular Weight Related | X | X | X | X | X | X | X | X | X | — | X | — | X |
| Substances-Non-Reducing CE-SDS | | | | | | | | | | | | | |
| Electrophoretic Purity-Non-reducing | X | X | X | X | X | X | X | X | X | X | X | X | X |
| SDS-PAGE | | | | | | | | | | | | | |
| Electrophoretic Purity-Reducing | X | X | X | X | X | X | X | X | X | X | X | X | X |
| SDS-PAGE | | | | | | | | | | | | | |
| Electrophoretic Purity-Reducing CE-SDS | X | X | X | X | X | X | X | X | X | — | X | — | X |
| Oxidized Forms by RP-UPLC | X | X | X | X | X | X | X | X | X | — | X | — | X |
| Deamidated Forms by SCX-HPLC | X | X | X | X | X | X | X | X | X | — | X | — | X |
| Sterility | X | — | X | — | X | — | X | — | X | — | X | — | X |
| Bacterial Endotoxins | X | — | X | — | X | — | X | — | X | — | X | — | X |

### Tables 34-41: Long Term Stability Results at 5±3° C. For IL-12 Fusion Protein DP Batches

Parameters tested include formulations' appearance, clarity and degree of opalescence, pH, osmolality, extractable volume, subvisible particles, protein concentration, specific activity, DNA binding activity, HMW related substances, electrophoretic purity, sterility, and bacterial endotoxins. Results of the long-term stability assessments and conditions for DP batches of IL-12 fusion protein are summarized in Tables 34, 35, 36, 37, 38, 39, 40, and 41 below.

TABLE 34
IL-12 fusion protein Long Term Stability Results at 5 ± 3° C. (Appearance)

| Test | Time | Batch Number | | | | |
|---|---|---|---|---|---|---|
| Parameter/Specification | (Months) | Y17A8880 | NH1D001 | NH1E001 | NH1G001 | NH1H001 |
| Appearance/ | 0 | Passed | Passed | Passed | Passed | Passed |
| Liquid, practically free of | 3 | Passed | Passed | Passed | Passed | Passed |
| visible particles | 6 | Passed | Passed | Passed | Passed | Passed |
| | 9 | Passed | Passed | Passed | Passed | Passed |
| | 12 | Passed | Passed | Passed | Passed | Passed |
| | 18 | Passed | Passed | Passed | Passed | Passed |
| | 24 | Passed | Passed | Passed | Passed | Passed |
| | 30 | Passed | Passed | Passed | Passed | Passed |
| | 36 | Passed | Passed | Passed | Passed | Passed |
| | 42 | Passed | Passed | Passed | NR | NR |
| | 48 | Passed | Passed | Passed | Passed | Passed |
| | 54 | Passed | Passed | Passed | NR | NR |
| | 60 | Passed | Passed | Passed | Passed | Passed |

NR: Not Required by the Stability Protocol

TABLE 35
IL-12 fusion protein Long Term Stability Results at 5 ± 3° C. (Clarity & Degree of Opalescence and pH)

| | Time | Batch Number | | | | |
|---|---|---|---|---|---|---|
| Test Parameter/Specification | (Months) | Y17A8880 | NH1D001 | NH1E001 | NH1G001 | NH1H001 |
| Clarity and Degree of Opalescence/ | 0 | 1.9 | 1.8 | 1.6 | 2.5 | 2.8 |
| NMT 6 NTU | 3 | NR | NR | NR | NR | NR |
| | 6 | 1.9 | 1.8 | 2.2 | 2.0 | 2.1 |
| | 9 | NR | NR | NR | NR | NR |
| | 12 | 1.7 | 1.9 | 1.8 | 2.1 | 1.9 |
| | 18 | NR | NR | NR | NR | NR |
| | 24 | 1.7 | 1.6 | 1.8 | 1.9 | 2.6 |
| | 30 | NR | NR | NR | NR | NR |
| | 36 | 1.9 | 2.2 | 1.7 | 1.5 | 1.5 |
| | 42 | 1.8 | 2.9 | 1.8 | NR | NR |
| | 48 | 1.8 | 2.5 | 2.1 | 1.6 | 1.8 |
| | 54 | 2.3 | 2.7 | 2.3 | NR | NR |
| | 60 | 2.0 | 1.6 | 1.7 | 1.2 | 1.1 |
| pH/ | 0 | 6.1 | 5.8 | 6.0 | 6.0 | 6.0 |
| 5.5-6.5 | 3 | NR | NR | NR | 6.0 | 6.0 |
| | 6 | 6.1 | 6.0 | 6.0 | 6.0 | 6.0 |
| | 9 | NR | NR | NR | 6.0 | 6.0 |
| | 12 | 6.1 | 6.0 | 5.9 | 6.0 | 6.0 |
| | 18 | NR | NR | NR | 6.0 | 6.0 |
| | 24 | 6.0 | 5.9 | 6.0 | 6.0 | 6.0 |
| | 30 | NR | NR | NR | 6.0 | 6.0 |
| | 36 | 6.0 | 6.0 | 6.0 | 6.0 | 5.9 |
| | 42 | 5.8 | 6.0 | 6.0 | NR | NR |
| | 48 | 6. | 5.9 | 6.0 | 6.0 | 6.0 |
| | 54 | 6.1 | 5.9 | 6.0 | NR | NR |
| | 60 | 6.0 | 6.0 | 5.9 | 6.0 | 6.0 |

NR: Not Required by the Stability Protocol

TABLE 36
IL-12 fusion protein Long Term Stability Results at 5 ± 3° C. (Osmolality and Extractable Volume)

| Test | Time | Batch Number | | | | |
|---|---|---|---|---|---|---|
| Parameter/Specification | (Months) | Y17A8880 | NH1D001 | NH1E001 | NH1G001 | NH1H001 |
| Osmolality/ | 0 | 247 | 252 | 287 | 286 | 323 |
| 240-350 mOsm/kg | 3 | NR | NR | NR | NR | NR |
| | 6 | 249 | 254 | 288 | 291 | 289 |
| | 9 | NR | NR | NR | NR | NR |
| | 12 | 249 | 254 | 295 | 286 | 308 |
| | 18 | NR | NR | NR | NR | NR |
| | 24 | 247 | 254 | 288 | 291 | 288 |
| | 30 | NR | NR | NR | NR | NR |
| | 36 | 251 | 257 | 323 | 291 | 288 |
| | 42 | 250 | 286 | 288 | NR | NR |
| | 48 | 248 | 253 | 286 | 292 | 281 |
| | 54 | 250 | 261 | 287 | NR | NR |
| | 60 | 248 | 256 | 287 | 286 | 287 |
| Extractable Volume/ | 0 | NR | NR | NR | 1.0 | 1.1 |
| 1.0-1.1 mL | 3 | NR | NR | NR | NR | NR |
| | 6 | NR | NR | NR | 1.1 | 1.1 |
| | 9 | NR | NR | NR | NR | NR |
| | 12 | NR | NR | NR | 1.1 | 1.1 |
| | 18 | NR | NR | NR | NR | NR |
| | 24 | NR | NR | NR | 1.1 | 1.1 |
| | 30 | NR | NR | NR | NR | NR |
| | 36 | NR | NR | NR | 1.1 | 1.1 |
| | 42 | NR | NR | NR | NR | NR |
| | 48 | NR | NR | NR | 1.0 | 1.0 |
| | 54 | NR | NR | NR | NR | NR |
| | 60 | NR | NR | NR | 1.1 | 1.1 |

NR: Not Required by the Stability Protocol

TABLE 37
IL-12 fusion protein Long Term Stability Results at 5 ± 3° C. (Subvisible Particles and Protein
Concentration)

| Test | Time | Batch Number | | | | |
|---|---|---|---|---|---|---|
| Parameter/Specification | (Months) | Y17A8880 | NH1D001 | NH1E001 | NH1G001 | NH1H001 |
| Particulate Contamination: | 0 | NR | 61 ≥ 10 μm | 4 ≥ 10 μm | 11 ≥ 10 μm | 13 ≥ 10 μm |
| Subvisible Particles/ | | | 0 ≥ 25 μm | 0 ≥ 25 μm | 1 ≥ 25 μm | 0 ≥ 25 μm |
| ≥10 mm: NMT 6000 particles | 3 | NR | NR | NR | NR | NR |
| ≥25 mm: NMT 600 particles | 6 | NR | NR | NR | 39 ≥ 10 μm | 42 ≥ 10 μm |
| (particles per container) | | | | | 1 ≥ 25 μm | 0 ≥ 25 μm |
| | 9 | NR | NR | NR | NR | NR |
| | 12 | NR | 5 ≥ 10 μm | 3 ≥ 10 μm | 48 ≥ 10 μm | 80 ≥ 10 μm |
| | | | 0 ≥ 25 μm | 0 ≥ 25 μm | 0 ≥ 25 μm | 1 ≥ 25 μm |
| | 18 | NR | NR | NR | NR | NR |
| | 24 | NR | 9 ≥ 10 μm | 8 ≥ 10 μm | 9 ≥ 10 μm | 8 ≥ 10 μm |
| | | | 0 ≥ 25 μm | 0 ≥ 25 μm | 0 ≥ 25 μm | 0 ≥ 25 μm |
| | 30 | NR | NR | NR | NR | NR |
| | 36 | NR | 26 ≥ 10 μm | 14 ≥ 10 μm | 10 ≥ 10 μm | 11 ≥ 10 μm |
| | | | 0 ≥ 25 μm | 0 ≥ 25 μm | 1 ≥ 25 μm | 1 ≥ 25 μm |
| | 42 | NR | 16 ≥ 10 μm | 20 ≥ 10 μm | NR | NR |
| | | | 0 ≥ 25 μm | 0 ≥ 25 μm | | |
| | 48 | NR | 12 ≥ 10 μm | 23 ≥ 10 μm | 16 ≥ 10 μm | 8 ≥ 10 μm |
| | | | 1 ≥ 25 μm | 0 ≥ 25 μm | 0 ≥ 25 μm | 0 ≥ 25 μm |
| | 54 | NR | 53 ≥ 10 μm | 18 ≥ 10 μm | NR | NR |
| | | | 3 ≥ 25 μm | 0 ≥ 25 μm | | |
| | 60 | NR | 34 ≥ 10 μm | 36 ≥ 10 μm | 56 ≥ 10 μm | 96 ≥ 10 μm |
| | | | 1 ≥ 25 μm | 1 ≥ 25 μm | 2 ≥ 25 μm | 4 ≥ 25 μm |
| Protein Concentration/ | 0 | 1.45 | 1.44 | 1.52 | 1.53 | 1.54 |
| 1.35-1.65 mg/mL | 3 | 1.43 | 1.48 | 1.49 | 1.51 | 1.54 |
| | 6 | 1.46 | 1.41 | 1.50 | 1.49 | 1.51 |
| | 9 | 1.49 | 1.49 | 1.51 | 1.52 | 1.56 |
| | 12 | 1.41 | 1.46 | 1.54 | 1.58 | 1.56 |
| | 18 | 1.45 | 1.42 | 1.58 | 1.50 | 1.54 |
| | 24 | 1.43 | 1.44 | 1.51 | 1.44 | 1.46 |
| | 30 | 1.43 | 1.42 | 1.51 | 1.42 | 1.44 |
| | 36 | 1.50 | 1.42 | 1.50 | 1.49 | 1.45 |
| | 42 | 1.42 | 1.42 | 1.48 | 1.53 | 1.53 |
| | 48 | 1.44 | 1.43 | 1.52 | 1.52 | 1.55 |
| | 54 | 1.45 | 1.44 | 1.44 | 1.53 | 1.55 |
| | 60 | 1.42 | 1.42 | 1.41 | 1.53 | 1.53 |

NR: Not Required by the Stability Protocol

TABLE 38
IL-12 fusion protein Long Term Stability Results at 5 ± 3° C. (Specific Activity)

| Test | Time | Batch Number | | | | |
|---|---|---|---|---|---|---|
| Parameter/Specification | (Months) | Y17A8880 | NH1D001 | NH1E001 | NH1G001 | NH1H001 |
| Specific Activity/ | 0 | 90284 | 94556 | 107342 | 91850 | 80642 |
| 65000-120000 U/mg | 3 | 86104 | NR | NR | 105874 | 90574 |
| | 6 | 82811 | 96162 | 101403 | 88751 | 90838 |
| | 9 | 74838 | 94109 | 94334 | 80601 | 74935 |
| | 12 | 101181 | 116368 | 80711 | 96622 | 95984 |
| | 18 | 79858 | 82590 | 76602 | 86508 | 92879 |
| | 24 | 91135 | 105074 | 102621 | 110848 | 92092 |
| | 30 | 90342 | 86789 | 92544 | 95204 | 103140 |
| | 36 | 89149 | 99953 | 95498 | 106192 | 98420 |
| | 42 | 92446 | 97301 | 85917 | 105163 | 95827 |
| | 48 | 98578 | 97838 | 88901 | 102273 | 91768 |
| | 54 | 94497 | 88207 | 110858 | 79357 | 92806 |
| | 60 | 93236 | 101986 | 95938 | 89339 | 78614 |

NR: Not Required by the Stability Protocol

TABLE 39
IL-12 fusion protein Long Term Stability Results at 5 ± 3° C. (DNA Binding Activity and HMW
SE-HPLC)

| | Time | Batch Number | | | | |
|---|---|---|---|---|---|---|
| Test Parameter/Specification | (Months) | Y17A8880 | NH1D001 | NH1E001 | NH1G001 | NH1H001 |
| DNA Binding Activity/ | 0 | 82 | 95 | 110 | 104 | 116 |
| 60-140% of Ref. Std. | 3 | 107 | 106 | 103 | 93 | 108 |
| | 6 | 96 | 109 | 95 | 93 | 105 |
| | 9 | 102 | 104 | 100 | 99 | 109 |
| | 12 | 102 | 92 | 113 | 97 | 99 |
| | 18 | 84 | 103 | 84 | 107 | 100 |
| | 24 | 101 | 96 | 103 | 92 | 116 |
| | 30 | 106 | 93 | 99 | 98 | 109 |
| | 36 | 102 | 102 | 87 | 99 | 108 |
| | 42 | 89 | 107 | 103 | NR | NR |
| | 48 | 102 | 95 | 100 | 114 | 119 |
| | 54 | 80 | 105 | 104 | 103 | 102 |
| | 60 | 92 | 94 | 107 | 98 | 112 |
| High Molecular Weight | 0 | 0.1 | 0.2 | 0.2 | 0.2 | 0.2 |
| Related Substances-SE-HPLC/ | 3 | 0.2 | 0.2 | 0.2 | 0.2 | 0.2 |
| NMT 3% | 6 | 0.2 | 0.3 | 0.2 | 0.2 | 0.2 |
| | 9 | 0.3 | 0.3 | 0.2 | 0.3 | 0.3 |
| | 12 | 0.3 | 0.3 | 0.2 | 0.3 | 0.3 |
| | 18 | 0.5 | 0.3 | 0.3 | 0.2 | 0.2 |
| | 24 | 0.3 | 0.4 | 0.3 | 0.3 | 0.3 |
| | 30 | 0.3 | 0.3 | 0.3 | 0.3 | 0.3 |
| | 36 | 0.3 | 0.4 | 0.3 | 0.3 | 0.3 |
| | 42 | 0.4 | 0.3 | 0.4 | NR | NR |
| | 48 | 0.3 | 0.3 | 0.3 | 0.3 | 0.3 |
| | 54 | 0.3 | 0.5 | 0.3 | 0.4 | 0.3 |
| | 60 | 0.4 | 0.4 | 0.3 | 0.4 | 0.4 |

NR: Not Required by the Stability Protocol

TABLE 40
IL-12 fusion protein Long Term Stability Results at 5 ± 3° C. (Electrophoretic Purity
Reducing and Non-reducing SDS-PAGE)

| Test | Time | Batch Number | | | | |
|---|---|---|---|---|---|---|
| Parameter/Specification | (Months) | Y17A8880 | NH1D001 | NH1E001 | NH1G001 | NH1H001 |
| Electrophoretic Purity- | 0 | 98 | 100 | 99 | 100 | 100 |
| Reducing SDS- | 3 | 98 | 100 | 99 | 98 | 99 |
| PAGE/ | 6 | 99 | 99 | 100 | 100 | 99 |
| NLT 90% | 9 | 100 | 99 | 100 | 99 | 100 |
| | 12 | 99 | 100 | 100 | 100 | 100 |
| | 18 | 99 | 99 | 100 | 98 | 99 |
| | 24 | 99 | 100 | 100 | 99 | 99 |
| | 30 | 99 | 100 | 97 | 98 | 98 |
| | 36 | 100 | 100 | 100 | 98 | 99 |
| | 42 | 99 | 98 | 98 | NR | NR |
| | 48 | 99 | 99 | 99 | 98 | 99 |
| | 54 | 99 | 98 | 99 | 98 | 99 |
| | 60 | 100 | 97 | 98 | 99 | 99 |
| Electrophoretic Purity- | 0 | 94 | 85 | 83 | 97 | 97 |
| Non-reducing SDS- | 3 | 96 | 89 | 86 | 95 | 95 |
| PAGE/ | 6 | 88 | 85 | 88 | 97 | 97 |
| NLT 80% | 9 | 87 | 91 | 92 | 96 | 95 |
| | 12 | 90 | 86 | 92 | 97 | 97 |
| | 18 | 89 | 91 | 91 | 94 | 95 |
| | 24 | 88 | 88 | 92 | 93 | 93 |
| | 30 | 87 | 86 | 90 | 94 | 92 |
| | 36 | 86 | 85 | 92 | 88 | 89 |
| | 42 | 85 | 91 | 85 | NR | NR |
| | 48 | 85 | 90 | 90 | 93 | 93 |
| | 54 | 87 | 81 | 91 | 93 | 95 |
| | 60 | 89 | 90 | 86 | 94 | 94 |

NA: Not Applicable;
NR: Not Required by the Stability Protocol

TABLE 41
IL-12 fusion protein Long Term Stability Results at 5 ± 3° C. (Sterility and Bacterial Endotoxins)

| Test | Time | Batch number | | | | |
|---|---|---|---|---|---|---|
| Parameter/Specification | (Months) | Y17A8880 | NH1D001 | NH1E001 | NH1G001 | NH1H001 |
| Sterility/ | 0 | Passed | Passed | Passed | Passed | Passed |
| Sterile | 3 | NR | NR | NR | NR | NR |
| | 6 | Passed | Passed | Passed | Passed | Passed |
| | 9 | NR | NR | NR | NR | NR |
| | 12 | Passed | Passed | Passed | Passed | Passed |
| | 18 | NR | NR | NR | NR | NR |
| | 24 | Passed | Passed | Passed | Passed | Passed |
| | 30 | NR | NR | NR | NR | NR |
| | 36 | Passed | Passed | Passed | Passed | Passed |
| | 42 | Passed | NR | NR | NR | NR |
| | 48 | Passed | Passed | Passed | Passed | Passed |
| | 54 | Passed | NR | NR | NR | NR |
| | 60 | Passed | Passed | Passed | Passed | Passed |
| Bacterial Endotoxins/ | 0 | <0.32 | 0.5 | 0.3 | <0.16 | <0.16 |
| NMT 15 EU/mg | 3 | NR | NR | NR | NR | NR |
| | 6 | 0.4 | 0.8 | 0.4 | <0.17 | <0.17 |
| | 9 | NR | NR | NR | NR | NR |
| | 12 | 0.8 | <0.32 | <0.25 | <0.16 | <0.16 |
| | 18 | NR | NR | NR | NR | NR |
| | 24 | 0.9 | <0.25 | 0.16 | <0.17 | <0.17 |
| | 30 | NR | NR | NR | NR | NR |
| | 36 | <0.32 | <0.25 | <0.17 | <0.17 | <0.17 |
| | 42 | <0.32 | NR | NR | NR | NR |
| | 48 | <0.32 | 0.17 | <0.16 | <0.16 | <0.16 |
| | 54 | <0.32 | NR | NR | NR | NR |
| | 60 | <0.25 | <0.18 | <0.18 | <0.16 | <0.16 |

NR: Not Required by the Stability Protocol

### Tables 42-43: Long Term Storage Stability of IL-12 Fusion Protein at −80° C.±10° C.

The IL-12 fusion protein formulation is also stable when stored frozen at −80° C.±10° C. for at least 48 months. The stability data for two different BDS lots; BNEA20002 and BNEA20003 are summarized in Tables 42 and 43, respectively.

TABLE 42
Long-Term Stability Results at −80 ± 10° C., Lot BNEA20002

| | | | Time Points (Months) | | | | | |
|---|---|---|---|---|---|---|---|---|
| Test | Method | Specification | 0 | 3 | 6 | 9 | 12 | 18 |
| Bacterial | Chromogenic | NMT 15 | <0.34 | — | <0.33 | — | <0.33 | — |
| Endotoxins | peptide | EU/mg | | | | | | |
| | kinetic | | | | | | | |
| | Method | | | | | | | |
| NANA | DMB-UPLC | Tentative limit: | 0.4 | 0.3 | 0.5 | 0.5 | 0.4 | 0.3 |
| Conc. | | NMT 1 | | | | | | |
| | | molNANA/mol | | | | | | |
| | | sample | | | | | | |
| | | (MTF) | | | | | | |
| NGNA | DMB-UPLC | NMT 10 | 5.3 | 4.9 | 7.1 | 5.9 | 6.1 | 4.9 |
| Conc. | | molNGNA/mol | | | | | | |
| | | sample | | | | | | |
| DNA- | ELISA | 60-140% of Ref. | 85 | 96 | 113 | 105 | 111 | 97 |
| Binding | | Standard | | | | | | |
| Assay | | | | | | | | |
| Biological | In vitro | 70-130% of | 97 | 102 | 114 | 108 | 93 | 98 |
| Activity | Bioassay | Ref. Standard | | | | | | |
| Clarity and | Nephelometry | NMT 6 NTU | 1.5 | 1.3 | 1.4 | 1.1 | 1.1 | 1.2 |
| degree of | | | | | | | | |
| opalescence | | | | | | | | |
| LMW | Non- | NMT 4% | 2.1 | 1.9 | 2.6 | 2.7 | 2.5 | 2.6 |
| related sub. | Reducing CE- | | | | | | | |
| (Fragments) | SDS | | | | | | | |
| Protein | O. D | 1.35-1.65 | 1.466 | 1.466 | 1.493 | 1.475 | 1.476 | 1.464 |
| Concentration | | mg/mL | | | | | | |
| Osmolality | Osmometry | 240-350 | 292 | 290 | 290 | 294 | 297 | 29 |
| | | mOsm/Kg | | | | | | |
| pH | Potentiometric | 5.5-6.5 | 5.9 | 6.0 | 5.9 | 6.0 | 6.0 | 6.0 |
| | determination | | | | | | | |
| Electrophoretic | Reducing CE- | NLT 94% | 96.8 | 97.8 | 96.4 | 96.7 | 96.1 | 96.3 |
| Purity | SDS | | | | | | | |
| (by CE- | | | | | | | | |
| SDS) | | | | | | | | |
| Biological | Reporter | To be reported | — | — | — | — | — | — |
| Activity_IL1 | Gene Assay | (% of Ref. Std.) | | | | | | |
| 2 Moiety | (RGA) | (MTF) | | | | | | |
| Biological | Reporter | To be reported | — | — | — | — | — | — |
| Activity of | Gene Assay | (% of Ref. Std.) | | | | | | |
| IL 12 Moiety | (RGA) | (MTF) | | | | | | |
| Arginine | RP-HPLC | Tentative limit: | 18.7 | 18.4 | 18.2 | 18.2 | 18.1 | 18.3 |
| Content | | 14-21 mg/mL | | | | | | |
| | | (MTF) | | | | | | |
| Polysorbate | RP-HPLC | Tentative limit: | 0.57 | 0.59 | 0.62 | 0.60 | 0.64 | 0.59 |
| 20 content | | 0.4-0.6 mg/mL | | | | | | |
| | | (MTF) | | | | | | |
| Oxidized | RP-HPLC | NMT 8% | 5.1 | 4.3 | 4.1 | 3.8 | 4.4 | 4.4 |
| Forms | | | | | | | | |
| Deamidated | SCX-HPLC | NMT 10% | 7.6 | 7.6 | 8.0 | 7.4 | 7.4 | 7.5 |
| Forms | | | | | | | | |
| HMW | SE-HPLC | NMT 3% | 0.3 | 0.2 | 0.2 | 0.3 | 0.3 | 0.3 |
| related | | | | | | | | |
| substances | | | | | | | | |
| Monomer | SE-HPLC | Tentative limit: | 99.4 | 99.4 | 99.4 | 99.4 | 99.4 | 99.3 |
| | | NLT 95% | | | | | | |
| | | (MTF) | | | | | | |
| Bioburden | Total Viable | NMT 1 CFU/10 | 0 | — | 0 | — | 0 | — |
| (TAMC) | Aerobic | mL | | | | | | |
| | Count | | | | | | | |
| Bioburden | Total Viable | NMT 1 CFU/10 | 0 | — | 0 | — | 0 | — |
| (TYMC) | Aerobic | mL | | | | | | |
| | Count | | | | | | | |
| Degree of | Visual | Not more | <7 | <7 | <7 | <7 | <7 | <7 |
| Coloration | inspection | intensely | | | | | | |
| | | coloured than | | | | | | |
| | | Ref.Sol. Y7 | | | | | | |
| Appearance | Visual | Liquid, | Passed | Passed | Passed | Passed | Passed | Passed |
| | inspection | practically free | | | | | | |
| | | from visible | | | | | | |
| | | particles | | | | | | |

Long-Term Stability Results at −80 ± 10° C., Lot BNEA20002

| | | | Time Points (Months) | | | | |
|---|---|---|---|---|---|---|---|
| Test | Method | Specification | 24 | 27 | 30 | 36 | 48 |
| Bacterial | Chromogenic | NMT 15 | <0.34 | — | — | <0.34 | <0.34 |
| Endotoxins | peptide | EU/mg | | | | | |
| | kinetic | | | | | | |
| | Method | | | | | | |
| NANA | DMB-UPLC | Tentative limit: | 0.4 | — | 0.4 | 0.4 | 0.4 |
| Conc. | | NMT 1 | | | | | |
| | | molNANA/mol | | | | | |
| | | sample | | | | | |
| | | (MTF) | | | | | |
| NGNA | DMB-UPLC | NMT 10 | 5.4 | — | 6.6 | 5.9 | 6.5 |
| Conc. | | molNGNA/mol | | | | | |
| | | sample | | | | | |
| DNA- | ELISA | 60-140% of Ref. | 113 | — | 105 | 109 | 105 |
| Binding | | Standard | | | | | |
| Assay | | | | | | | |
| Biological | In vitro | 70-130% of | 93 | — | 104 | 97 | 93 |
| Activity | Bioassay | Ref. Standard | | | | | |
| Clarity and | Nephelometry | NMT 6 NTU | 1.4 | — | 1.3 | 1.4 | 1.6 |
| degree of | | | | | | | |
| opalescence | | | | | | | |
| LMW | Non- | NMT 4% | 2.5 | — | 2.5 | 1.8 | 1.6 |
| related sub. | Reducing CE- | | | | | | |
| (Fragments) | SDS | | | | | | |
| Protein | O. D | 1.35-1.65 | 1.469 | — | 1.452 | 1.461 | 1.450 |
| Concentration | | mg/mL | | | | | |
| Osmolality | Osmometry | 240-350 | 292 | — | 282 | 285 | 288 |
| | | mOsm/Kg | | | | | |
| pH | Potentiometric | 5.5-6.5 | 6.0 | — | 6.0 | 6.0 | 6.0 |
| | determination | | | | | | |
| Electrophoretic | Reducing CE- | NLT 94% | 96.3 | — | 97.5 | 96.4 | 96.3 |
| Purity | SDS | | | | | | |
| (by CE- | | | | | | | |
| SDS) | | | | | | | |
| Biological | Reporter | To be reported | — | 97 | 99 | — | — |
| Activity_IL1 | Gene Assay | (% of Ref. Std.) | | | | | |
| 2 Moiety | (RGA) | (MTF) | | | | | |
| Biological | Reporter | To be reported | — | — | — | 100 | 99 |
| Activity of | Gene Assay | (% of Ref. Std.) | | | | | |
| IL 12 Moiety | (RGA) | (MTF) | | | | | |
| Arginine | RP-HPLC | Tentative limit: | 21.2 | — | 18.1 | 17.7 | 18.8 |
| Content | | 14-21 mg/mL | | | | | |
| | | (MTF) | | | | | |
| Polysorbate | RP-HPLC | Tentative limit: | 0.59 | — | 0.59 | 0.53 | 0.59 |
| 20 content | | 0.4-0.6 mg/mL | | | | | |
| | | (MTF) | | | | | |
| Oxidized | RP-HPLC | NMT 8% | 4.5 | — | 4.4 | 4.2 | 4.5 |
| Forms | | | | | | | |
| Deamidated | SCX-HPLC | NMT 10% | 7.3 | — | 7.2 | 7.3 | 7.1 |
| Forms | | | | | | | |
| HMW | SE-HPLC | NMT 3% | 0.2 | — | 0.2 | 0.2 | 0.3 |
| related | | | | | | | |
| substances | | | | | | | |
| Monomer | SE-HPLC | Tentative limit: | 99.2 | — | 99.5 | 99.5 | 99.3 |
| | | NLT 95% | | | | | |
| | | (MTF) | | | | | |
| Bioburden | Total Viable | NMT 1 CFU/10 | 0 | — | — | 0 | 0 |
| (TAMC) | Aerobic | mL | | | | | |
| | Count | | | | | | |
| Bioburden | Total Viable | NMT 1 CFU/10 | 0 | — | — | 0 | 0 |
| (TYMC) | Aerobic | mL | | | | | |
| | Count | | | | | | |
| Degree of | Visual | Not more | <1 | — | <7 | <7 | <7 |
| Coloration | inspection | intensely | | | | | |
| | | coloured than | | | | | |
| | | Ref.Sol. Y7 | | | | | |
| Appearance | Visual | Liquid, | Passed | — | Passed | Passed | Passed |
| | inspection | practically free | | | | | |
| | | from visible | | | | | |
| | | particles | | | | | |

TABLE 43
Long-Term Stability Results at −80 ± 10° C., Lot BNEA20003

| | | | Time Points (Months) | | | | | |
|---|---|---|---|---|---|---|---|---|
| Test | Method | Specification | 0 | 3 | 6 | 9 | 12 | 18 |
| Bacterial | Chromogenic | NMT 15 EU/mg | <0.34 | — | <0.33 | — | <0.33 | — |
| Endotoxins | peptide kinetic | | | | | | | |
| | Method | | | | | | | |
| NANA Conc. | DMB-UPLC | Tentative | 0.4 | 0.3 | 0.4 | 0.4 | 0.4 | 0.3 |
| | | limit: NMT | | | | | | |
| | | 1 molNANA/ | | | | | | |
| | | molsample | | | | | | |
| | | (MTF) | | | | | | |
| NGNA Conc. | DMB-UPL C | NMT 10 | 5.5 | 4.5 | 5.9 | 6.0 | 6.0 | 4.9 |
| | | molNGNA/ | | | | | | |
| | | molsample | | | | | | |
| DNA-Binding | ELISA | 60-140% of Ref. | 108 | 108 | 100 | 103 | 113 | 108 |
| Assay | | Standard | | | | | | |
| Biological | In vitro | 70-130 % of Ref. | 102 | 112 | 111 | 116 | 109 | 103 |
| Activity | Bioassay | Standard | | | | | | |
| Clarity and | Nephelometry | NMT 6 NTU | 1.5 | 1.4 | 1.3 | 1.2 | 1.1 | 1.3 |
| degree of | | | | | | | | |
| opalescence | | | | | | | | |
| LMW | Non-Reducing | NMT 4% | 1.9 | 2.0 | 2.5 | 2.7 | 2.4 | 2.6 |
| related sub. | CE-SDS | | | | | | | |
| (Fragments) | | | | | | | | |
| Protein | O. D | 1.35-1.65 mg/mL | 1.473 | 1.470 | 1.493 | 1.478 | 1.479 | 1.472 |
| Concentration | | | | | | | | |
| Osmolality | Osmometry | 240-350 | 292 | 293 | 294 | 290 | 294 | 294 |
| | | mOsm/Kg | | | | | | |
| pH | Potentiometric | 5.5-6.5 | 6.0 | 6.1 | 5.8 | 6.0 | 6.0 | 6.0 |
| | determination | | | | | | | |
| Electrophoretic | Reducing | NLT 94% | 95.2 | 97.5 | 96.7 | 96.6 | 96.5 | 96.4 |
| Purity | CE-SDS | | | | | | | |
| (by CE-SDS) | | | | | | | | |
| Biological | Reporter Gene | To be reported | — | — | — | — | — | — |
| Activity_IL 12 | Assay (RGA) | (% of Ref. Std.) | | | | | | |
| Moiety | | (MTF) | | | | | | |
| Biological | Reporter Gene | To be reported | — | — | — | — | — | — |
| Activity | Assay (RGA) | (% of | | | | | | |
| of IL 12 | | Ref. Std.) (MTF) | | | | | | |
| Moiety | | | | | | | | |
| Arginine | RP-HPLC | Tentative limit: | 18.4 | 18.2 | 18.0 | 18.0 | 17.9 | 18.6 |
| Content | | 14-21 mg/mL | | | | | | |
| | | (MTF) | | | | | | |
| Polysorbate | RP-HPLC | Tentative limit: | 0.52 | 0.53 | 0.56 | 0.53 | 0.59 | 0.50 |
| 20 content | | 0.4-0.6 mg/mL | | | | | | |
| | | (MTF) | | | | | | |
| Oxidized | RP-HPLC | NMT 8% | 4.9 | 4.1 | 3.8 | 4.0 | 4.3 | 4.0 |
| Forms | | | | | | | | |
| Deamidated | SCX-HPLC | NMT 10% | 5.1 | 5.1 | 5.0 | 4.6 | 4.6 | 4.6 |
| Forms | | | | | | | | |
| HMW related | SE-HPLC | NMT 3% | 0.2 | 0.2 | 0.2 | 0.2 | 0.2 | 0.2 |
| substances | | | | | | | | |
| Monomer | SE-HPLC | Tentative | 99.4 | 99.4 | 99.4 | 99.5 | 99.4 | 99.2 |
| | | limit: NLT | | | | | | |
| | | 95% (MTF) | | | | | | |
| Bioburden | Total Viable | NMT 1 | 0 | — | 0 | — | 0 | — |
| (TAMC) | Aerobic Count | CFU/10 mL | | | | | | |
| Bioburden | Total Viable | NMT 1 | 0 | — | 0 | — | 0 | — |
| (TYMC) | Aerobic Count | CFU/10 mL | | | | | | |
| Degree of | Visual | Not more | <7 | <7 | <7 | <7 | <7 | <7 |
| Coloration | inspection | intensely | | | | | | |
| | | colored than | | | | | | |
| | | Ref.Sol. Y7 | | | | | | |
| Appearance | Visual | Liquid, | Passed | Passed | Passed | Passed | Passed | Passed |
| | inspection | practically | | | | | | |
| | | free from | | | | | | |
| | | visible particles | | | | | | |

Long-Term Stability Results at −80 ± 10° C., Lot BNEA20003

| | | | Time Points (Months) | | | | |
|---|---|---|---|---|---|---|---|
| Test | Method | Specification | 24 | 27 | 30 | 36 | 48 |
| Bacterial | Chromogenic | NMT 15 EU/mg | <0.34 | — | — | <0.34 | <0.34 |
| Endotoxins | peptide kinetic | | | | | | |
| | Method | | | | | | |
| NANA Conc. | DMB-UPLC | Tentative | 0.4 | — | 0.4 | 0.4 | 0.4 |
| | | limit: NMT | | | | | |
| | | 1 molNANA/ | | | | | |
| | | molsample | | | | | |
| | | (MTF) | | | | | |
| NGNA Conc. | DMB-UPL C | NMT 10 | 6.2 | — | 7.4 | 5.5 | 6.9 |
| | | molNGNA/ | | | | | |
| | | molsample | | | | | |
| DNA-Binding | ELISA | 60-140% of Ref. | 104 | — | 94 | 109 | 111 |
| Assay | | Standard | | | | | |
| Biological | In vitro | 70-130 % of Ref. | 113 | — | 104 | 110 | 104 |
| Activity | Bioassay | Standard | | | | | |
| Clarity and | Nephelometry | NMT 6 NTU | 1.4 | — | 1.3 | 1.4 | 1.7 |
| degree of | | | | | | | |
| opalescence | | | | | | | |
| LMW | Non-Reducing | NMT 4% | 2.6 | — | 2.6 | 1.9 | 2.0 |
| related sub. | CE-SDS | | | | | | |
| (Fragments) | | | | | | | |
| Protein | O. D | 1.35-1.65 mg/mL | 1.475 | — | 1.454 | 1.465 | 1.450 |
| Concentration | | | | | | | |
| Osmolality | Osmometry | 240-350 | 289 | — | 285 | 288 | 292 |
| | | mOsm/Kg | | | | | |
| pH | Potentiometric | 5.5-6.5 | 6.0 | — | 6.0 | 6.0 | 6.0 |
| | determination | | | | | | |
| Electrophoretic | Reducing | NLT 94% | 96.5 | — | 97.7 | 96.4 | 96.6 |
| Purity | CE-SDS | | | | | | |
| (by CE-SDS) | | | | | | | |
| Biological | Reporter Gene | To be reported | — | 103 | 106 | — | — |
| Activity_IL 12 | Assay (RGA) | (% of Ref. Std.) | | | | | |
| Moiety | | (MTF) | | | | | |
| Biological | Reporter Gene | To be reported | — | — | — | 102 | 98 |
| Activity | Assay (RGA) | (% of | | | | | |
| of IL 12 | | Ref. Std.) (MTF) | | | | | |
| Moiety | | | | | | | |
| Arginine | RP-HPLC | Tentative limit: | 18.2 | — | 18.1 | 17.5 | 18.7 |
| Content | | 14-21 mg/mL | | | | | |
| | | (MTF) | | | | | |
| Polysorbate | RP-HPLC | Tentative limit: | 0.52 | — | 0.53 | 0.60 | 0.53 |
| 20 content | | 0.4-0.6 mg/mL | | | | | |
| | | (MTF) | | | | | |
| Oxidized | RP-HPLC | NMT 8% | 4.2 | — | 4.0 | 4.0 | 4.3 |
| Forms | | | | | | | |
| Deamidated | SCX-HPLC | NMT 10% | 4.7 | — | 4.5 | 4.7 | 4.4 |
| Forms | | | | | | | |
| HMW related | SE-HPLC | NMT 3% | 0.2 | — | 0.2 | 0.2 | 0.2 |
| substances | | | | | | | |
| Monomer | SE-HPLC | Tentative | 99.2 | — | 99.5 | 99.5 | 99.3 |
| | | limit: NLT | | | | | |
| | | 95% (MTF) | | | | | |
| Bioburden | Total Viable | NMT 1 | 0 | — | — | 0 | 0 |
| (TAMC) | Aerobic Count | CFU/10 mL | | | | | |
| Bioburden | Total Viable | NMT 1 | 0 | — | — | 0 | 0 |
| (TYMC) | Aerobic Count | CFU/10 mL | | | | | |
| Degree of | Visual | Not more | <7 | — | <7 | <7 | <7 |
| Coloration | inspection | intensely | | | | | |
| | | colored than | | | | | |
| | | Ref.Sol. Y7 | | | | | |
| Appearance | Visual | Liquid, | Passed | — | Passed | Passed | Passed |
| | inspection | practically | | | | | |
| | | free from | | | | | |
| | | visible particles | | | | | |

Although the invention has been described with reference to the above examples, it will be understood that modifications and variations are encompassed within the spirit and scope of the invention. Accordingly, the invention is limited only by the following claims.

### Example 12

### Freezing and Thawing the Pharmaceutical Formulation

### Protocol for Freezing and Thawing the IL-12 Fusion Formulation:

To test the stability of the protein formulation, the formulation drug substance or product is subjected to 3 consecutive freeze-thaw cycles. The formulation vials are first analyzed at TO in terms of appearance, protein content (mg/mL), and monomer and high molecular weight content/percentages utilizing SE-HPLC. The vials are then transferred and stored at −70±10° C. for a minimum of 2 hours before removing and thawing for at least 2 hours at ambient temperatures (+20±5° C.). This is repeated for 2 additional cycles.

To determine stability of the formulation, post each freeze-thaw cycle, appearance will be analyzed to ensure the formulation remains clear and free of visible particles, protein content will be determined and compared to the TO reading, and the monomer and high molecular weight percentages will be compared to the TO run. In each SE-HPLC analytical sequence, system suitability samples as well as control/standard samples are included for reference in all sequences.

1. A pharmaceutical formulation comprising:
(a) a fusion protein comprising an interleukin-12 (IL-12) peptide or fragment thereof and an antibody or a fragment thereof;
(b) a citrate buffer;
(c) a stabilizer;
(d) a sugar; and
(e) a surfactant.

2. The pharmaceutical formulation of claim 1, wherein the citrate buffer comprises about 1 mM to 20 mM of citric acid.

3. (canceled)

4. The pharmaceutical composition of claim 1, wherein the stabilizer comprises arginine, methionine or niacinamide.

5. (canceled)

6. The pharmaceutical formulation of claim 1, wherein the stabilizer comprises about 50 mM to 200 mM L-arginine.

7. (canceled)

8. The pharmaceutical formulation of claim 1, wherein the sugar comprises sucrose, lactose or mannitol.

9. The pharmaceutical formulation of claim 1, wherein the sugar comprises sucrose.

10-11. (canceled)

12. The pharmaceutical formulation of claim 1, wherein the surfactant comprises polysorbate 20 (PS20), polysorbate 80 (PS80) or poloxamer 188 (P188).

13. The pharmaceutical formulation of claim 1, therein the surfactant comprises PS20.

14-27. (canceled)

28. The pharmaceutical formulation of claim 1, comprising:
(a) from about 1 mM to 20 mM citric acid;
(b) from about 50 mM to 200 mM L-arginine;
(c) from about 10 mM to 200 mM sucrose; and
(d) from about 0.01% (w/w) to 0.5% (w/w) PS20.

29. The pharmaceutical formulation of claim 1, comprising:
(a) about 10 mM citric acid,
(b) about 100 mM L-arginine;
(c) about 100 mM sucrose; and
(d) about 0.05% (w/w) PS20.

30. The pharmaceutical formulation of claim 1, wherein the formulation comprises a pH of about 6.0.

31. The pharmaceutical formulation of claim 1, wherein the fusion protein is stable for at least 60 months at a temperature from about 2° C. to 8° C.

32-38. (canceled)

39. A method of treating cancer comprising administering to a subject a therapeutically effective amount of the pharmaceutical formulation of claim 1, thereby treating cancer.

40-41. (canceled)

42. The method of claim 39, wherein the pharmaceutical formulation comprises:
(a) from about 1 mM to 20 mM citric acid;
(b) from about 50 mM to 200 mM L-arginine;
(c) from about 10 mM to 200 mM sucrose; and
(d) from about 0.01% (w/w) to 0.5% (w/w) PS20.

43. The method of claim 39, wherein the pharmaceutical formulation comprises:
(a) about 10 mM citric acid,
(b) about 100 mM L-arginine;
(c) about 100 mM sucrose; and
(d) about 0.05% (w/w) PS20.

44. The method of claim 39, wherein the fusion protein comprises an IL-12 heterodimer fused to a heavy chain of an IgG1 monoclonal antibody.

45-60. (canceled)

61. A method of enhancing T cell infiltration and proliferation in a tumor microenvironment comprising administering to a subject a therapeutically effective amount of the pharmaceutical formulation of claim 1, thereby enhancing T cell infiltration and proliferation.

62. The method of claim 61, wherein the pharmaceutical formulation comprises:
(a) from about 1 mM to 20 mM citric acid;
(b) from about 50 mM to 200 mM L-arginine;
(c) from about 10 mM to 200 mM sucrose; and
(d) from about 0.01% (w/w) to 0.5% (w/w) PS20.

63. The method of claim 61, wherein the pharmaceutical formulation comprises:
(a) about 10 mM citric acid,
(b) about 100 mM L-arginine;
(c) about 100 mM sucrose; and
(d) about 0.05% (w/w) PS20.

64. The method of claim 61, wherein the fusion protein comprises an IL-12 heterodimer fused to a heavy chain of an IgG1 monoclonal antibody.

65-66. (canceled)

67. The pharmaceutical formulation of claim 1, wherein the fusion protein comprises an IL-12 heterodimer fused to a heavy chain of an IgG1 monoclonal antibody.


SEQUENCE LISTING (61 sequences)

SEQ ID NO: 1 - protein, 7 aa, synthetic construct
SSEEEDE

SEQ ID NO: 2 - protein, 8 aa, synthetic construct
SSEEEDEE

SEQ ID NO: 3 - protein, 8 aa, synthetic construct
SEEEDESS

SEQ ID NO: 4 - protein, 10 aa, synthetic construct
SEEEDESEED

SEQ ID NO: 5 - protein, 15 aa, synthetic construct
GQAEPDRAHYNIVTF

SEQ ID NO: 6 - protein, 20 aa, synthetic construct
MHGDTPTLHEYMLDLQPETT

SEQ ID NO: 7 - protein, 17 aa, synthetic construct
LLMGTLGIVCPICSQKP

SEQ ID NO: 8 - protein, 21 aa, synthetic construct
ELQTTIHDIILECVYCKQQLL

SEQ ID NO: 9 - protein, 24 aa, synthetic construct
KSSELQTTIHDIILECVYCKQQLL

SEQ ID NO: 10 - protein, 23 aa, synthetic construct
KSSMHGDTPTLHEYMLDLQPETT

SEQ ID NO: 11 - protein, 20 aa, synthetic construct
KSSLLMGTLGIVCPICSQKP

SEQ ID NO: 12 - protein, 18 aa, synthetic construct
KSSGQAEPDRAHYNIVTF

SEQ ID NO: 13 - protein, 40 aa, synthetic construct
YLAIVYLIALAVCQVRRKNYGQLDIFPARDKYHPMSEYAL

SEQ ID NO: 14 - protein, 10 aa, synthetic construct
YLAIVYLIAL

SEQ ID NO: 15 - protein, 10 aa, synthetic construct
YLIALAVCQV

SEQ ID NO: 16 - protein, 10 aa, synthetic construct
ALWGQDVTSV

SEQ ID NO: 17 - protein, 9 aa, synthetic construct
YLSYTNPAV

SEQ ID NO: 18 - protein, 9 aa, synthetic construct
YLAPPAHGV

SEQ ID NO: 19 - protein, 9 aa, synthetic construct
YLDTRPAPV

SEQ ID NO: 20 - protein, 10 aa, synthetic construct
YLAIVTLIAL

SEQ ID NO: 21 - protein, 10 aa, synthetic construct
YLIALAVCQV

SEQ ID NO: 22 - protein, 9 aa, synthetic construct
YLAPPAHGV

SEQ ID NO: 23 - protein, 9 aa, synthetic construct
YLDTRPAPV

SEQ ID NO: 24 - protein, 9 aa, synthetic construct
YLSYTNPAV

SEQ ID NO: 25 - protein, 10 aa, synthetic construct
ALFIVYLIAK

SEQ ID NO: 26 - protein, 9 aa, synthetic construct
SLFRSPYEK

SEQ ID NO: 27 - protein, 10 aa, synthetic construct
KYHPMSEYAL

SEQ ID NO: 28 - protein, 9 aa, synthetic construct
KYTNPAVAL

SEQ ID NO: 29 - protein, 10 aa, synthetic construct
ALAIVYLIAL

SEQ ID NO: 30 - protein, 10 aa, synthetic construct
YLIALAVCQC

SEQ ID NO: 31 - protein, 9 aa, synthetic construct
STAPPAHGV

SEQ ID NO: 32 - protein, 9 aa, synthetic construct
APDTRPAPG

SEQ ID NO: 33 - protein, 9 aa, synthetic construct
SLSYTNPAV

SEQ ID NO: 34 - protein, 10 aa, synthetic construct
ALAIVYLIAL

SEQ ID NO: 35 - protein, 9 aa, synthetic construct
STDRSPYEK

SEQ ID NO: 36 - protein, 10 aa, synthetic construct
TYHPMSEYPT

SEQ ID NO: 37 - protein, 9 aa, synthetic construct
SYTNPAVAA

SEQ ID NO: 38 - protein, 9 aa, synthetic construct
FVFLRNFSL

SEQ ID NO: 39 - protein, 9 aa, synthetic construct
FLRNFSLMV

SEQ ID NO: 40 - protein, 20 aa, synthetic construct
MQMFPPSPLFFFLQLLKQSS

SEQ ID NO: 41 - protein, 20 aa, synthetic construct
FFLQLLKQSSRRLEHTFVFL

SEQ ID NO: 42 - protein, 20 aa, synthetic construct
RRLEHTFVFLRNFSLMLLRG

SEQ ID NO: 43 - protein, 20 aa, synthetic construct
RNFSLMLLRGIGKKRRATRF

SEQ ID NO: 44 - protein, 18 aa, synthetic construct
IGKKRRATRFWDPRRGTP

SEQ ID NO: 45 - protein, 38 aa, synthetic construct
MQMFPPSPLFFFLQLLKQSSRRLEHTFVFLRNFSLMVL

SEQ ID NO: 46 - protein, 32 aa, synthetic construct
FVFLRNFSLMVLRGIGKKRRATRFWDPRRGTP

SEQ ID NO: 47 - protein, 45 aa, synthetic construct
SEEEDESSMQMFPPSPLFFFLQLLKQSSRRLEHTFVFLRNFSLMV

SEQ ID NO: 48 - protein, 42 aa, synthetic construct
SEEEDESEEDFVFLRNFSLMVLRGIGKKRRATRFWDPRRGTP

SEQ ID NO: 49 - protein, 46 aa, synthetic construct
SEEEDESSMQMFPPSPLFFFLQLLKQSSRRLEHTFVFLRNFSLMLV

SEQ ID NO: 50 - protein, 10 aa, synthetic construct
YMLDLQPETT

SEQ ID NO: 51 - protein, 9 aa, synthetic construct
LLMGTLGIV

SEQ ID NO: 52 - protein, 13 aa, synthetic construct
KSSYMLDLQPETT

SEQ ID NO: 53 - protein, 12 aa, synthetic construct
KSSLLMGTLGIV

SEQ ID NO: 54 - protein, 233 aa, synthetic construct
MELPVRLLVLMFWIPASLSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 55 - protein, 109 aa, synthetic construct
SSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKVTVL

SEQ ID NO: 56 - protein, 663 aa, synthetic construct
MELPVRLLVLMFWIPASLSQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSATATPGAANLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 57 - protein, 117 aa, synthetic construct
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSS

SEQ ID NO: 58 - protein, 19 aa, synthetic construct
MELPVRLLVLMFWIPASLS

SEQ ID NO: 59 - protein, 326 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKDNTEGRVFTDKTSATVICRKNASISVRAQDRYYSSSWSEWASVPCS

SEQ ID NO: 60 - protein, 10 aa, Homo sapiens
KSKREKKDRV

SEQ ID NO: 61 - protein, 8 aa, synthetic construct
KDNTEGRV